(19) United States
(12) Patent Application Publication
(10) Pub. No.: US 2025/0101067 A1
(43) Pub. Date: Mar. 27, 2025

(54) MODIFIED HUMAN CYTOMEGALOVIRUS PROTEINS

(71) Applicant: GLAXOSMITHKLINE BIOLOGICALS SA, Rixensart (BE)

(72) Inventors: Marco BIANCUCCI, Rockville, MD (US); Newton Muchugu WAHOME, Rockville, MD (US)

(73) Assignee: GLAXOSMITHKLINE BIOLOGICALS SA, Rixensart (BE)

(21) Appl. No.: 18/832,219

(22) PCT Filed: Jan. 19, 2023
(86) PCT No.: PCT/IB2023/050457

§ 371 Date: Jul. 23, 2024

Related U.S. Application Data
(60) Provisional application No. 63/304,193, filed on Jan. 28, 2022.
(60) Provisional application No. 63/350,123, filed on Jun. 8, 2022.

(51) Int. Cl. C07K 14/005; A61K 39/00; C07K 14/39; C12N 9/88

(52) U.S. Cl. CPC C07K 14/005; C07K 14/39; C12N 9/88; A61K 39/00; A61K 2039/55505; A61K 2039/55566; A61K 2039/55577; C07K 2319/02; C07K 2319/21; C07K 2319/50; C07K 2319/73; C12N 2710/16122; C12N 2710/16134

(57) ABSTRACT

Modified HCMV gB proteins in a pre-fusion conformation, compositions comprising such proteins, and uses thereof.

### FIELD OF THE INVENTION

This invention relates to recombinant human cytomegalovirus (HCMV) gB proteins.

### BACKGROUND OF THE INVENTION

Human cytomegalovirus (HCMV) is a double stranded DNA virus of the β-herpesvirus subfamily. Congenital HCMV infection is the leading cause of hearing loss, vision loss and neurological disability. In addition, HCMV causes life-threatening illnesses in individuals with a compromised immune system, such as AIDS patients or transplant recipients. A vaccine against HCMV is needed.

At least twenty viral proteins are expressed on the HCMV envelope, including glycoproteins B, L and O (gB, gL and gO, respectively), and proteins UL128, UL130 and UL131A. The glycoprotein gB trimer and gH/gL heterodimer form a core fusion machinery. HCMV cell entry consists of a two-step mechanism: receptor binding and membrane fusion. While the gH/gL/gO and gH/gL/UL128/UL130/UL131A (corresponding to the HCMV pentamer) oligomers recognize their cognate receptors on cell membranes and regulate cell tropism, the gB trimer mediates membrane fusion and acts as a viral fusogen. Based on their structural properties, viral fusogens are divided into three classes, with HCMV gB belonging to class III. Viral fusogens are anchored into the viral envelope and reside in a metastable pre-fusion state on the viral membrane. Upon triggering, viral fusogens undergo a conformational rearrangement, switching to a stable post-fusion conformation and facilitating the merging of the viral lipid envelope with the host cell membrane.

There is a need to develop stable HCMV gB proteins that are in a pre-fusion conformation and use such proteins for the development of a vaccine against HCMV.

### SUMMARY OF THE INVENTION

The present inventors designed HCMV gB sequences that produce a gB protein in pre-fusion conformation and, importantly, produce a purified HCMV gB protein in a stabilized pre-fusion conformation. To do so, the (1) Membrane Proximal Region (MPR), transmembrane domain, and cytoplasmic domains are inactivated by truncation (e.g., by deleting the amino acid residues c-terminal to the residue corresponding to D703 of SEQ ID NO: 1) and (2) replaced with a heterologous trimerization domain that promotes the formation of stable trimers (such as a GCN4 Leucine Zipper domain), also (3) at least one, at least two, at least three, at least four, at least five, or at least six amino acid substitutions to cysteine (C) are introduced (e.g., introducing three pairs of recombinant cysteines resulting in three new, recombinant disulfide bonds being formed within the mutant molecule: S269C with L656C, N524C with M684C, and D217C with S587C, numbered according to SEQ ID NO: 1). For clarity, by replacing the amino acids c-terminal to the residue corresponding to D703 of SEQ ID NO: 1, the membrane proximal region (MPR) is dramatically truncated (nearly deleted) and both the transmembrane domain and the cytoplasmic domain are completely deleted (i.e., full length Domains I-V remain present). See FIG. 1. These modifications are sufficient to produce purified HCMV gB proteins in a stabilized pre-fusion conformation. This is surprising because other attempts to truncate HCMV gB MPR, add a GCN4 domain there, and also substitute certain amino acid residues to cysteine failed to produce a purified protein stabilized in pre-fusion conformation (CHI et al. WO2021260510, where “stabilized” in this context means that post-fusion conformation is not formed).

Without wishing to be bound by theory, the present inventors believe that the combination of (1) inactivating the MPR, transmembrane domain, and cytoplasmic domains by truncation (e.g., removing the residues c-terminal to any one of D699, K700, V701, V702, and D703 numbered according to SEQ ID NO: 1), (2) operably linking a heterologous trimerization domain c-terminal to Domain V, and (3) introducing recombinant disulfide bonds (via one, two, three, four, five, or six amino acid substitutions to cysteine) disrupts the transition of the viral gB protein from pre-fusion to post-fusion conformation and therefore is sufficient to produce a purified HCMV gB protein in a stabilized pre-fusion conformation (Towne, Merlin, or AD169 strains).

In certain embodiments, the present disclosure provides an HCMV gB protein with an amino acid sequence comprising at least one, at least two, at least three, at least four, at least five, or at least six recombinant Cysteine (C) residues. In certain other aspects the HCMV gB is characterized by having a heterologous trimerization domain operably linked c-terminal to the residue corresponding to any one of 698, 699, 700, 701, 702, 703, 704, 705, 706, 707, or 708 of SEQ ID NO: 1. In certain aspects, the HCMV gB amino acid sequence comprises a Domain I amino acid sequence that is at least 190 total residues in length, a Domain II amino acid sequence that is at least 83 total residues in length, a Domain III amino acid sequence that is at least 76 total residues in length, a Domain IV amino acid sequence that is at least 87 total residues in length, and a Domain V amino acid sequence that is at least 30 total residues in length. In still other embodiments, the HCMV gB amino acid sequence comprises (except for the recombinant one, two, three, four, five, or six cysteine residues), residues that correspond to residues 89-648 of SEQ ID NO: 1 and at least 30 consecutive residues from 649-698 (inclusive of endpoints) of SEQ ID NO: 1. In certain aspects, the HCMV gB amino acid sequence comprises (except for the recombinant one, two, three, four, five, or six cysteine residues), residues that correspond to residues 89-698 of SEQ ID NO: 1. In certain aspects, an HCMV gB provided herein has an amino acid sequence comprising (numbered according to SEQ ID NO: 1) a recombinant cysteine residue at residue 239 (e.g., SEQ ID NOs: 56, 57, 58, and 59); a recombinant cysteine residue at residue 156 (e.g., SEQ ID NOs: 60, 61, 62, and 63); a recombinant cysteine residue at residues 217 and 587 (e.g., SEQ ID NOs: 64, 65, 66, and 67); a recombinant cysteine residue at residues 524 and 684 (e.g., SEQ ID NOs: 36, 37, 38, 39); a recombinant cysteine residue at residues 100 and 267 (e.g., SEQ ID NOs: 24, 25, 26, and 27); a recombinant cysteine residue at residues 269 and 656 (e.g., SEQ ID NOs: 34 and 35); a recombinant cysteine residue at residues 251 and 614 (e.g., SEQ ID NOs: 30 and 31); a recombinant cysteine residue at residues 100, 251, and 614 (e.g., SEQ ID NOs: 28 and 29); a recombinant cysteine residue at residues 100, 269, and 656 (e.g., SEQ ID NOs: 32 and 33); a recombinant cysteine residue at residues 269, 524, 656, and 684 (e.g., SEQ ID NOs: 52, 53, 54, and 55); a recombinant cysteine residue at residues 100, 267, 524, and 684 (e.g., SEQ ID NOs: 44, 45, 46, and 47); a recombinant cysteine residue at residues 251, 524, 614, and 684 (e.g., SEQ ID NOs: 50 and 51); a recombinant cysteine residue at residues 217, 524, 587, and 684 (e.g., SEQ ID NOs: 42 and 43); a recombinant cysteine residue at residues 100, 217, 524, 587, and 684 (e.g., SEQ ID NOs: 40 and 41); a recombinant cysteine residue at residues 100, 251, 524, 614, and 684 (e.g., SEQ ID NOs: 48 and 49); a recombinant Cysteine (C) at the residues corresponding to 217, 269, 524, 587, 656, and 684 (e.g., SEQ ID NOs: 3, 4, and 11).

In certain aspects, the present disclosure provides an HCMV gB protein with an amino acid sequence comprising the recombinant amino acid residues (numbered according to SEQ ID NO: 1): (A) T98, G100, V103, K181, G183, Y200, C239, G240, V255, V267, L268, 1273, D281, A314, D350, T358, T360, 1367, I376, A377, E384, 1428, P433, K493, 1516, W517, R535, W541, L542, 1548, S549, D570, Q583, F591, T621, D650, L663, E696, 1697, N704, Q710, S714, and D717 (e.g., SEQ ID NOs: 56 and 57); (B) C239 (e.g., SEQ ID NOs: 58 and 59); (C) T98, G100, V103, C156, K181, G183, Y200, G240, E256, V267, L268, 1273, D281, A314, D350, T360, I367, V376, A377, P384, 1428, P433, K493, 1516, W517, R535, W541, L542, 1548, S549, D570, Q583, F591, T621, D650, L663, E696, N704, Q710, S714, D716, and D717 (e.g., SEQ ID NOs: 60 and 61); (D) C156 (e.g., SEQ ID NOs: 62 and 63); (E) N98, G100, R181, G183, C217, G240, V255, L268, D281, D350, T360, 1367, P433, E493, W517, 1548, S549, C587, F591, D650, N704, S714, and D717 (e.g., SEQ ID NOs: 64 and 65); (F) C217 and C587 (e.g., SEQ ID NOs: 66 and 67); (G) G100, F103, Y114, 1142, G183, Y200, C217, V248, V267, A314, V355, 1367, T377, F382, 1428, P433, T493, Q497, 1516, W517, R535, A547, S549, D570, R583, S585, C587, Q624, D650, N652, and I694 (e.g., SEQ ID NOs: 20 and 21); (H) C217 and C587 (e.g., SEQ ID NOs: 22 and 23); (I) G100, F103F, 1142, G183, V267, H337, V355, 1367, F428, P433, A493, W517, C524, A547, S549, D570, R583, Q624, C684, and I694 (e.g., SEQ ID NOs: 36 and 37); (J) C524 and C684 (e.g., SEQ ID NOs: 38 and 39); (K) C100, F103, 1142, G183, C267, A314, H337, V355, 1367, T377, F428, P433, A493, W517, A547, S549, D570, R583, S583, Q624, S650, N652, and I694 (e.g., SEQ ID NOs: 24 and 25); (L) C100 and C267 (e.g., SEQ ID NOs: 26 and 27); (M) C267 and C656 (e.g., SEQ ID NOs: 34 and 35); (N) C251 and C614 (e.g., SEQ ID NOs: 30 and 31); (O) C100C, F103, 1142, G183, V267, C251, V355, 1367, F428, P433, K493, W517, A547, S549, D570, R583, S583, C614, Q624, S650, N652, and 1694 (e.g., SEQ ID NOs: 28 and 29); (P) C100, F103, C267, I367I, 1428, P433, K493, W517, R583, Q624, and C656 (e.g., SEQ ID NOs: 32 and 33); (Q) G100, F103, 1142, G183, V267, C267, H337, V355, 1367, F428, P433, A493, W517, C524, A547, S549, D570, R583, E585, Q624, S650, N652, C656, C684, and I694 (e.g., SEQ ID NOs: 52 and 53); (R) C267, C524, C656, and C684 (e.g., SEQ ID NOs: 54 and 55); (S) C100, F103, 1142, G183, C267, H337, V355, 1367, F428, P433, K493, W517, C524, A547, S549, D570, R583, S583, Q624, S650, N652, C684, and 1694 (e.g., SEQ ID NOs: 44 and 45); (T) C100, C267, C524, and C684 (e.g., SEQ ID NOs: 46 and 47); (U) C251, C524, C614, and C684 (e.g., SEQ ID NOs: 50 and 51); (V) C217, C524, C587, and C684 (e.g., SEQ ID NOs: 42 and 43); (W) C100, F103, 1142, G183, C217, V267, A314, H337, V355, 1367, T377, F428, P433, K493, W517, C524, A547, S549, D570, R583, E585, C587, Q624, S650, N652, C684, 1694, and R695 (e.g., SEQ ID NOs: 40 and 41); (X) C100, F103, 1142, G183, V267, C251, H337, V355, 1367, F428, P433, K493, W517, C524, A547, S549, D570, R583, E585, C614, Q624, S650, N652, C684, and I694 (e.g., SEQ ID NOs: 48 and 49); or (Y) C217, C269, C524, C587, C656, and C684 (e.g., SEQ ID NOs: 3, 4, and 11).

In an aspect of the present disclosure, the HCMV gB protein comprises an amino acid sequence with at least 90%, 91%, 92%, 93%, 94%, 95%, 96%, 97%, 98%, 99%, or 100% sequence identity to any one of SEQ ID NOS: 3, 4, 11, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67. In certain aspects of the disclosure, the HCMV gB protein comprises an amino acid sequence with the recombinant residue(s) and at least 90%, 91%, 92%, 93%, 94%, 95%, 96%, 97%, 98%, 99%, or 100% sequence identity to SEQ ID NOS: 3, 4, 11, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67.

In one embodiment, the amino acid sequence of the HCMV gB protein does not comprise a recombinant proline residue. In another embodiment, the amino acid sequence of the HCMV gB protein comprises (or consists of) only one recombinant proline residue at the position corresponding to residue 433 of SEQ ID NO: 1. In another embodiment, the amino acid sequence of the HCMV gB protein comprises (or consists of) only two recombinant proline residues at the positions corresponding to residues 433 and 384 of SEQ ID NO: 1.

In an aspect of the invention, the HCMV gB protein is in pre-fusogenic conformation. In a further aspect of the invention, the HCMV gB protein is purified and in pre-fusogenic conformation. In an aspect of the invention, the majority of proteins obtained by protein expression (e.g., in vivo expression) of the modified HCMV gB sequences of this invention are in the pre-fusion conformation. In these aspects it may be clarified that the protein is not bound to a heterologous molecule (such as bound to an antibody or antibody fragment, or bound to a helper molecule such as the inhibitor used by Liu et al. (2021 Sci. Adv. 7: eabf3178) or CHI et al. (WO2021260510).

One aspect of the present invention provides a modified HCMV gB protein comprising a heterologous trimerization domain operably linked c-terminal to the residue corresponding to any one of 698, 699, 700, 701, 702, and 703 numbered according to SEQ ID NO: 1 (specifically any one of E698, D699, K700, V701, V702, and D703 numbered according to SEQ ID NO: 1). The modified gB protein herein may be truncated, i.e., not comprise the native amino acid residues c-terminal to the residue corresponding to 698, 699, 700, 701, 702, or 703 respectively, numbered according to SEQ ID NO: 1 (specifically, does not comprise the native amino acid residues corresponding to 699-907, 700-907, 701-907, 702-907, 703-907, or 704-907 of SEQ ID NO: 1, respectively). Such native amino acid residues form essentially all of (or all of) the MPR domain, all of the transmembrane domain, and all of the cytoplasmic domain (see FIG. 1). In this context, “essentially all of” means at least 60% of the amino acid sequence.

In another aspect, the modified HCMV gB protein has an amino acid linker sequence between the residue corresponding to 698, 699, 700, 701, 702, or 703, numbered according to SEQ ID NO: 1 (i.e., the c-terminal-most residue of the truncated MPR or, in the case of a deleting all of the MPR, the c-terminal-most residue of the Domain V) and the GCN4 domain. Suitable linker sequences include a single amino acid (such as a single proline, serine, glycine or alanine residue), two amino acid residues (such as two proline (P), serine(S), glycine (G) or alanine (A) residues, or any combination thereof), or any short amino acid sequence that functions as a linker sequence.

In certain aspects, the HCMV gB comprises a heterologous trimerization domain. In certain further aspects the heterologous trimerization domain is a GCN4 Leucine Zipper domain (GCN4); a suitable GCN4 domain comprises or consists of the amino acid sequence provided as SEQ ID NO: 2, 12, 13, 14, 15, 16, or 17. In certain other aspects, the heterologous trimerization domain is an aldolase sequence (meaning a nucleic acid or amino acid sequence corresponding to that which is naturally present within an aldolase). Additional multimerization domains that drive the symmetric self-assembly of trimers of soluble proteins are known in the art (Engel et al. 2000 Matrix Biology 19.4:283-288), and may be used in the present invention. Examples of suitable multimerization domains include: (1) the GCN4 leucine zipper (Harbury et al. 1993 Science 262:1401-1407); (2) the phage T4 fibritin foldon (Miroshnikov et al. 1998 Protein Eng 11:329-414 (3) collagen (McAlinden et al. 2003 Biol Chem 278:42200-42207; see also US patent publication 2017/0182151), (4) De novo designed trimeric oligomers (Boyken et al. 2016 Science 352:680-687), and (5) trimeric aldolase structures (e.g., from a 2-keto-3-deoxy-6-phosphogluconate (KDPG) aldolase (Fullerton et al. 2006 Bioorg Med Chem 14 (9): 3002-3010)). A heterologous trimerization domain for use herein may be an aldolase sequence, for example, an aldolase sequence that comprises or consists of the amino acid sequence SEQ ID NO: 18 or 19.

In certain aspects, the modified HCMV gB protein comprises a substitution modification of one or more furin cleavage site amino acid, including but not limited to R457S and R460S substitutions (numbered per SEQ ID NO: 1).

In certain aspects, the modified HCMV gB protein comprises a substitution modification of one or more fusion loop amino acids, including but not limited to substitution modifications at FL1 residues 155-157 and/or FL2 residues 240-242 (numbered per SEQ ID NO: 1); such as Y155G, I156H, H157R, W240F, L241F, and Y242H (numbered per SEQ ID NO: 1); or such as I156H, H157R, and W240R.

In other aspects, the modified HCMV gB protein comprises a purification tag (e.g., a 6-histidine purification tag) and may also comprise a cleavage linker (e.g., a TEV cleavage linker).

In another aspect, the modified HCMV gB protein is operably linked to a carrier. In certain aspects, the carrier is a nanoparticle (e.g., a virus like particle (VLP) or self-assembling protein nanoparticle such as a lumazine synthase nanoparticle, ferritin nanoparticle, or an aldolase-based nanoparticle). In the context of an HCMV gB polynucleotide sequence, certain aspects provide for a HCMV gB nucleotide sequence that is operably linked (e.g., genetically fused) to a heterologous nucleotide sequence that encodes a carrier (e.g., a nanoparticle) or a monomer that, after expression, will form a nanoparticle carrier (in the case of a trimeric protein nanoparticle carrier, e.g., will trimerize after expression to form the trimeric protein nanoparticle).

In another aspect is provided isolated nucleic acids comprising a polynucleotide sequence encoding the modified HCMV gB proteins herein. The nucleic acid may be an RNA. The RNA may be mRNA. The RNA may be self-amplifying RNA (“saRNA”, also referred to as self-replicating RNA or self-amplifying/replicating mRNA), optionally an alphavirus replicon. An alphavirus replication particle (VRP) may comprise the alphavirus replicon.

In certain aspects is provided an immunogenic composition comprising a designed HCMV gB protein or designed HCMV gB nucleic acid. Such compositions may comprise at least one additional antigenic protein, or nucleic acid molecule encoding such at least one additional antigenic protein. In certain aspects, the additional antigenic protein is gO, gH, gL, pUL128, pUL130, pUL131, pp65, IE1, an antigenic fragment thereof, or a combination thereof; optionally those of the HCMV pentamer (i.e., gH or a gH fragment, gL, pUL128, pUL130, and pUL131).

In another aspect is provided immunogenic compositions comprising a modified HCMV gB protein, nucleic acid, or VRP of herein. Immunogenic compositions may comprise an adjuvant, and the adjuvant may comprise aluminum salt, a TLR7 agonist, TLR4 agonist, MPL, 3D-MPL, saponin, or an oil-in-water emulsion. Immunogenic compositions herein may comprise a modified HCMV gB protein and at least one additional protein or nucleic acid antigen. The at least one additional antigen may be an HCMV protein, or immunogenic fragment thereof, such as gO, gH, gL, pUL128, pUL130, pUL131, pp65, IE1, or a combination thereof (such as gH, gL, pUL128, pUL130, and pUL131 which together form the HCMV pentamer).

In one aspect is provided a recombinant vector comprising a nucleic acid described herein. In certain aspects, the recombinant vector is suitable for prophylactic or therapeutic use (such as, for example, the recombinant vector delivered to a subject via administration of a composition comprising a lipid nanoparticle and the recombinant vector).

In a further aspect is provided an isolated host cell comprising a nucleic acid as described herein. The nucleic acid may comprise a polynucleotide that is DNA and such DNA may be stably incorporated into the genomic DNA of the host cell. The host cell may be a mammalian cell. The mammalian cell may be a CHO cell or HEK-293 cell.

In an aspect is provided a cell culture comprising a host cell as described herein.

In another aspect is provided processes of producing a modified HCMV gB protein comprising culturing a host cell under suitable conditions, and optionally then collecting the modified gB protein from the cultured host cell(s), and optionally purifying the modified gB protein.

In further aspects is provided processes of producing a population of host cells, comprising culturing host cells under suitable conditions for expression of a modified HCMV gB protein as described herein and optionally purifying the modified gB protein, wherein a majority of the purified modified gB proteins are in pre-fusogenic (pre-fusion) conformation.

In one aspect is provided a population of host cells wherein a majority of the modified HCMV gB proteins expressed from said host cells are in pre-fusogenic conformation.

In another aspect is provided an isolated antibody, or antigen-binding fragment thereof, specific for a modified HCMV gB protein described herein. A pharmaceutical composition may comprise such antibody, or antigen-binding fragment thereof.

Another aspect provides uses of the modified HCMV gB proteins, nucleic acids, VRPs, immunogenic compositions, vectors, antibodies or antibody fragments, or pharmaceutical compositions described herein for the prevention or treatment of HCMV infection, for inducing an immune response against HCMV, for the manufacture of a medicament for inducing an immune response against HCMV, or for the manufacture of a medicament for inhibiting HCMV entry into a cell.

Also provided are methods of inhibiting membrane fusion of HCMV to a cell, inhibiting HCMV entry into a cell, or inducing an immune response against HCMV in a subject, comprising contacting the cell with the modified HCMV gB proteins, nucleic acids, VRPs, immunogenic compositions, vectors, antibodies or antibody fragments, or pharmaceutical compositions described herein.

In another aspect is provided kits for identifying the presence of a modified HCMV gB protein within a composition, for the purification of a modified HCMV gB protein from a composition, for detecting a modified HCMV gB protein in pre-fusion conformation or a non-post-fusion conformation, or for the prevention or treatment of HCMV virus infection; comprising the antibody or antigen-binding fragment as described herein. Such kits may include instructions for using the antibody or antigen-binding fragment thereof.

### BRIEF DESCRIPTION OF THE FIGURES

FIG. 1 is a schematic representation of the HCMV gB protein from the Merlin strain. Abbreviations therein are as follows: “TM” meaning transmembrane domain; “Cyto” meaning cytoplasmic domain; “SP” meaning signal peptide; “MPR” meaning membrane proximal region; “I” meaning Domain I; “II” meaning Domain II; “III” meaning Domain III; “IV” meaning Domain IV; and “V” meaning Domain V. The amino acid numbering and residues are based on HCMV Merlin strain gB sequence SEQ ID NO: 1 (see also Chandramouli et al., 2015 Nat. Commun. 6 (8176): 1-12; and WO 2016/092460 (corresponding to US 2018/0265551)). Circled ‘G’ represents N glycosylation site. Fusion loops, Furin Cleavage Site, and DIII Coiled-coil region are indicated according to the HCMV Merlin strain gB sequence (SEQ ID NO:1).

FIG. 2A and FIG. 2B provide an alignment spanning the Domain III Coiled-coil (DIIIcc) Region and Membrane Proximal Region (MPR) amino acid residues of (top to bottom) HCMV Merlin strain gB (SEQ ID NO: 1), and HCMV AD169 strain gB (SEQ ID NO: 6). Residue numbering is provided with respect to SEQ ID NO: 1 and, in this way, demonstrates what is meant by an HCMV AD169 gB amino acid residue that “corresponds to” a residue “numbered according to SEQ ID NO: 1”.

### DETAILED DESCRIPTION

### 1. Overview

The crystal structure of the post-fusion HCMV gB ectodomain revealed a trimeric oligomer with five defined structural domains (Chandramouli et al. 2015 Nat. Comm. 6 (8176): 1-12 and Burke & Heldwein 2015 PLOS Path. DOI: 10.1371).

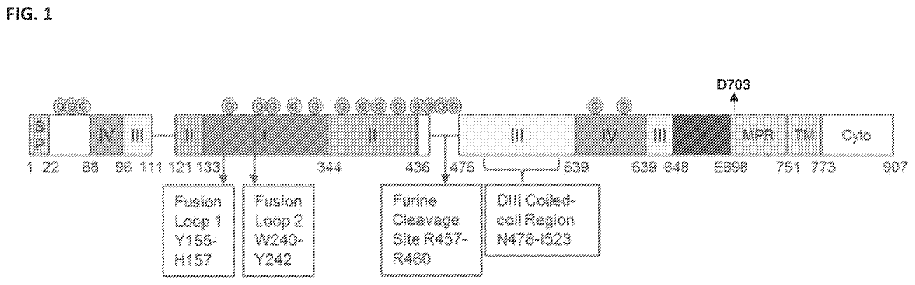

A HCMV vaccine containing soluble post-fusion gB adjuvanted with MF59 has been reported as inducing a potent immunogenic response against gB in a Phase I trial (Frey et al. 1999 J. Infect. Dis. 180 (5): 1700-1703; Li et al. 2017 NPJ Vaccines 2 (36) DOI: 10.1038/s41541-017-0038-0). In addition, different Phase II studies with post-fusion HCMV gB+MF59 reported 50% efficacy for prevention of primary HCMV infection in postpartum women, 43% efficacy in seronegative adolescent girls and reduced viremia in solid-transplant-recipients with seropositive organ donors (Pass et al. 2009 New Engl. J. Med. 360:1191-1199; Griffiths et al. 2011 Lancet 377 (9773): 1256-1263; Bernstein et al. 2016 Vaccine 34 (3): 313-319). Recently, two independent studies reported that protection conferred by HCMV post-fusion gB subunit vaccine is not dependent on neutralizing antibodies (Nelson et al. 2018 PNAS 115 (24): 6267-6272; Baraniak et al. 2018 PNAS 115 (24): 6273-6278), possibly indicating that vaccine-induced neutralizing responses should target epitopes in the pre-fusion form. A study on whole HCMV virions purports to show a low-resolution model of the pre-fusogenic state based on cryo-electron tomography and resolved to 21 Å (EMDB ID EMD-9328 with primary publication by Si et al. 2018 PLOS Path. 14 (12): e1007452). This supposed pre-fusion gB model has a Christmas tree-like conformation, where the apical part seems to belong to the coiled-coil region of Domain III (which is usually buried in the post-fusion conformation) (FIG. 1). But the low-resolution model by Si Z. et al. does not allow for structural design of an HCMV gB molecule (e.g., one that is not in post-fusion conformation) and, in particular, structural design of a stable HCMV gB molecule in the pre-fusion conformation. Using a fusion inhibitor and a chemical cross-linker, a team obtained, and have now published, higher resolution structural details of HCMV gB protein (Towne strain) in pre-fusion conformation (see Liu et al. 2021 Sci. Adv. 7: eabf3178). Based on that information, that team have since disclosed a plethora of potential modifications that may stabilize a purified HCMV gB protein in pre-fusion conformation (Dormitzer et al. US 2020/0247853 and CHI et al. WO2021260510), but only a few mutants have actually been analyzed and of those few: none result in a purified HCMV gB protein that is stabilized in a pre-fusion conformation (“stabilized” meaning in this context that the purified HCMV gB protein does not form the post-fusion conformation). In particular, CHI et al. (WO2021260510) tested whether certain combinations of cysteine mutations would stabilize a purified HCMV gB protein in pre-fusion conformation (there, Towne strain), but the pairs of cysteine mutations that they analyzed all failed for that purpose (post-fusion conformation was obtained). CHI et al. (WO2021260510) state that they could only obtain a purified HCMV gB stabilized in pre-fusion conformation by complete deletion of Domain V (see Example 7 at Tables 13-15 and accompanying text).

As is provided herein, the inventors have discovered that a specific combination of modifications introduced into a HCMV gB sequence results in a purified HCMV gB protein stabilized in a pre-fusion conformation (in particular, where the modified HCMV gB protein has full length Domains I-V).

Other modifications as described herein or as known by the art (e.g., modification of a furin cleavage site, a fusion loop(s), or addition of a purification tag) may also be included in the modified HCMV gB proteins of the present invention.

### 2. Aspects and Definitions

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. The term “at least one” refers to one or more.

Additionally, all numerical limitations given with respect to concentrations or levels of a substance, such as solution component concentrations or ratios thereof, and reaction conditions such as temperatures, pressures, and cycle times are intended to be approximate. Unless specified otherwise, where a numerical range is provided, it is inclusive, i.e., the endpoints are included.

“Pre-Fusogenic” and “Pre-Fusion” are used interchangeably herein.

“Recombinant Cysteine residues” as used herein refers to cysteines that do not occur in nature. Such recombinant cysteine residues are introduced into a protein sequence (e.g., into an HCMV gB protein). This introduction can be by way of insertion of a cysteine residue or substitution of a residue with a cysteine residue. Moreover, “Recombinant Proline residues” as used herein refers to prolines that do not occur in nature and are introduced into a protein sequence.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215:403-410 and Altschul, et al., 1977, Nucleic Acids Res. 3389-3402). Software for performing BLAST analyses is publicly available through the National Center for Biotechnology Information website.

HCMV gB is an envelope glycoprotein B having numerous roles, one of which is the involvement in the fusion of the cytomegalovirus with host cells. It is encoded by the UL55 gene of HCMV genome. The size of the native form of HCMV gB depends on the size of the open reading frame (ORF) which may vary a little according to the strain. For example, the ORF of AD169 strain, which is 2717 bp long, encodes a full length gB of 906 amino acids, whereas the ORF of Towne and Merlin strains encode a full length gB of 907 amino acids. Although the present invention is applicable to gB proteins originating from any HCMV strain, in order to facilitate its understanding, when referring to amino acid positions in the present specification, the numbering is given in relation to the amino acid sequence of the gB protein of SEQ ID NO:1 originating from the clinical isolate Merlin strain, unless otherwise stated. The present invention is not, however, limited to the HCMV Merlin strain.

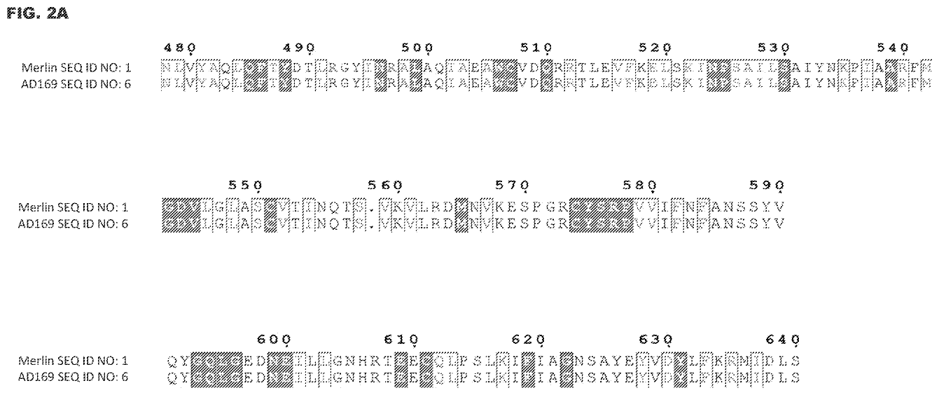

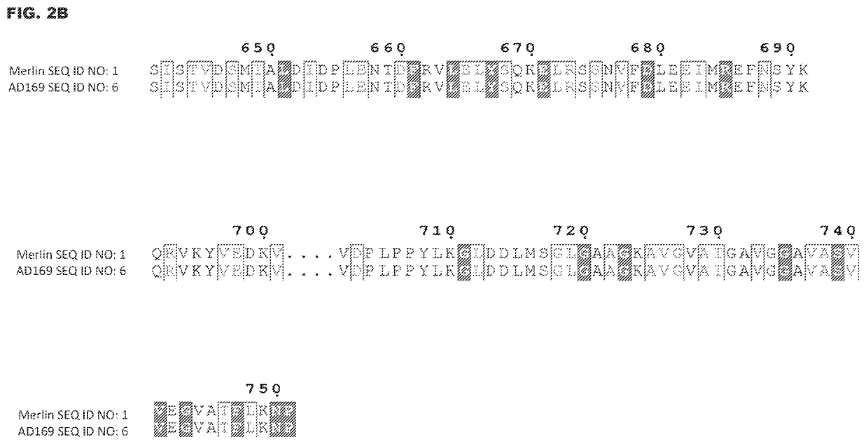

Therefore, when referring to “comparable” or “corresponding” amino acid positions or specific amino acids in a gB protein of any other HCMV strain (also relevant in the context of nucleic acids), such “comparable” or “corresponding” residues (or nucleic acids) can be determined by those of ordinary skill in the art using known information (see FIG. 5 of Deckers et al. 2009 Vir. J. 6 (210): 1-12; Foglierini et al. 2019 Front. Micro. 19 (1005): 1-8; WO 2012/049317 (corresponding to US 2013/0216613); and WO 2016/092460 (corresponding to US 2018/0265551; and Burke & Heldwein 2015 PLOS Path. DOI: 10.1371) and by sequence alignment using readily available and well-known alignment algorithms (such as BLAST, using default settings; ClustalW2, using default settings; or algorithm disclosed by Corpet, Nucleic Acids Research, 1998, 16 (22): 10881-10890, using default parameters). An example of using sequence alignment to determine which residues “correspond to” a referenced residue is provided at FIGS. 2A and 2B (see also FIG. 2 of Burke & Heldwein 2015 PLOS Path. DOI: 10.1371). Accordingly, when referring to a “HCMV gB protein”, it is to be understood as a HCMV gB protein from any strain. The actual residue location number and residue identity may have to be adjusted for gB proteins from HCMV other than HCMV Merlin strain, depending on the actual sequence alignment. (See also Tables 1-2 and FIGS. 2A and 2B). A gB residue that “corresponds to” a residue in a different HCMV gB (e.g., a different strain) may have a different position number, may be a different amino acid, or both (see Tables 1-2 and FIGS. 2A-2B). If a residue location is given a number “according to” a particular amino acid sequence (e.g., “numbered according to SEQ ID NO: 1” or “numbered per SEQ ID NO: 1”), it means that the residue aligns to (i.e., it “corresponds to”) that numbered location of the referenced amino acid sequence (following the example, the residue aligns to that numbered location of SEQ ID NO: 1).

TABLE 1
Summary of Corresponding
MPR Residues

| HCMV Merlin | HCMV AD169 |
|---|---|
| Strain gB SEQ | Strain gB SEQ |
| ID NO: 1 | ID NO: 6 |
| UniProtKB | UniProtKB |
| F5HB53 | P06473 |
| MPR residue | MPR residue |
| D703 | D702 |

A gB residue that “corresponds to” a residue in a different HCMV gB strain may have a different position number, may be a different amino acid, or both (see also FIGS. 2A-2B).

TABLE 2
Summary of Positions for Introducing
a (Recombinant) Cysteine

| | HCMV AD169 |
|---|---|
| HCMV Merlin | Strain gB |
| Strain gB | SEQ ID NO: 6 |
| SEQ ID NO: 1 | UniProtKB |
| UniProtKB F5HB53 | P06473 |
| T100 | T100 |
| I156 | I156 |
| D217 | D217 |
| T239 | T239 |
| G251 | G251 |
| A267 | A267 |
| S269 | S269 |
| G271 | G271 |
| N524 | N523 |
| S587 | S586 |
| P614 | P613 |
| L656 | L655 |
| M684 | M683 |

As yet a further example, it would be well understood by the art that, for example, the HCMV AD-169 strain gB Fusion Loop 1 (FL1) sequence ¹⁵⁵YIY¹⁵⁷ “corresponds to” or is “comparable to” the HCMV Merlin strain gB FL1 sequence ¹⁵⁵ YIH¹⁵⁷ (Y¹⁵⁷ from AD-169 strain gB corresponding to H¹⁵⁷ of Merlin strain gB) (compare WO 2012/049317 (corresponding to US 2013/0216613) and WO 2016/092460 (corresponding to US 2018/0265551). It is known in the art that at least one Recombinant Cysteine could be inserted in the protein sequence. Thus, Cysteine could be introduced in the amino acid sequence be either substitution or insertion.

Orientation within a polypeptide is generally recited in an N-terminal to C-terminal direction, defined by the orientation of the amino and carboxy moieties of individual amino acids. Polypeptides are translated from the N-terminal or amino-terminus towards the C-terminal or carboxy-terminus.

The native form of HCMV Merlin strain gB is set forth in FIG. 1 and generally contains, in the N-terminal to C-terminal direction of the protein, (i) an amino acid signal sequence or signal peptide (here designated as residues 1-22 of SEQ ID NO: 1, but designated by some as residues 1-24 of SEQ ID NO: 1 (see UniProtKB Accession No. F5HB53)), known to be involved in the polypeptide intracellular trafficking including targeting the polypeptide towards secretion, followed by (ii) a region called the leader sequence, (iii) an extracellular domain containing five structural domains (I-V) and an endoproteolytic furin cleavage site (with Domain I comprising two fusion loops), (iv) a Membrane Proximal Region (MPR), (v) a transmembrane domain and (vi) a C-terminal cytoplasmic domain. For the purposes herein, Domain I (DI) corresponds to residues 134-344 of SEQ ID NO: 1 (inclusive is 210 AA), Domain II (DII) corresponds to residues 122-133 and 345-436 of SEQ ID NO: 1 (inclusive is 103 AA), Domain III (DIII) corresponds to residues 97-111, 476-539, and 640-648 of SEQ ID NO: 1 (inclusive is 96 AA), Domain IV (DIV) corresponds to residues 89-96 and 540-639 of SEQ ID NO: 1 (inclusive is 107 AA), and Domain V (DV) corresponds to residues 649-698 of SEQ ID NO: 1 (inclusive is 50 AA). It would be well understood in the art that the labeling of what residues constitute Domains I-V herein is not dispositive and others may label certain residues differently (e.g., when analyzing a sequence from a different strain of HCMV gB and/or when analyzing two- or three-dimensional structures). See, e.g., Liu et al. 2021 Sci. Adv. 7: eabf3178 at FIG. 2 and accompanying text. To that end, it would not be surprising for there to be a shift of 1, 2, 3, 4, 5, 6, 7, 8, 9, or even 10 residues as compared to others' designation of what specific HCMV gB amino acid (AA) residues are within any one of Domains I-V (see CHI et al. WO 2021/610510 pages 79-80). Again, for the purposes herein, the residue labels above are used.

In general, the HCMV gB protein forms a monomeric trimer (comprising three gB proteins, also referred to as subunits) that can be used as an antigen against HCMV. However, the monomeric trimer comprises an exposed hydrophobic surface, which can cause significant problems in both antigen production and purification. For example, the hydrophobic surface can cause aggregation of recombinantly produced gB protein (e.g., two monomeric trimers can form a dimeric trimer via the hydrophobic surface, which may cause production problems). The hydrophobic surface also causes the monomeric gB trimer to adhere to the host cell (e.g., to cell membrane, ER membrane, other hydrophobic proteins, aggregated proteins, etc.). To reduce aggregation and/or increase protein secretion of a gB protein, one or more residues within either or both of Fusion Loop 1 and Fusion Loop 2 corresponding to residues 155-157 and 240-242, respectively, of SEQ ID NO: 1 may be modified (e.g., by amino acid residue substitution) (see Chandramouli et al. 2015 Nat. Comm. 6 (8176): 1-12; WO 2012/049317 (corresponding to US 2013/0216613) and WO 2016/092460 (corresponding to US 2018/0265551). It may therefore be specified that the HCMV gB comprises a substitution modification of one or more fusion loop amino acids, including but not limited to substitution modifications at FL1 residues 155-157 and/or FL2 residues 240-242 (numbered per SEQ ID NO: 1); such as Glycine (G) at the residue corresponding to 155 of SEQ ID NO: 1, Histidine (H) at the residue corresponding to 156 of SEQ ID NO: 1, Arginine (R) at the residue corresponding to 157 of SEQ ID NO: 1, Phenylalanine (F) at the residue corresponding to 240 of SEQ ID NO: 1, (F) at the residue corresponding to 241 of SEQ ID NO: 1, and (H) at the residue corresponding to 242 of SEQ ID NO: 1. It may be otherwise specified that the HCMV gB comprises Histidine (H) at the residue corresponding to 156 of SEQ ID NO: 1, Arginine (R) at the residue corresponding to 157 of SEQ ID NO: 1, and (R) at the residue corresponding to 240 of SEQ ID NO: 1.

Proteins (or polypeptides) herein may be modified to carry further modifications, such as for instance, modifications at endoproteolytic cleavage sites so that said sites are made ineffectual. For example, the furin cleavage site located around amino acids 457 to 460 of the sequence set forth in SEQ ID NO:1, or at a corresponding position in other gB proteins originating from different HCMV strains, may be modified. See WO 2012/049317 (corresponding to US 2013/0216613) and WO 2016/092460 (corresponding to US 2018/0265551).

The term “fragment,” in reference to a polypeptide, refers to a portion (that is, a subsequence) of a polypeptide. An “immunogenic fragment” of a gB protein refers to a fragment that retains at least one immunogenic epitope (e.g., a predominant immunogenic epitope or a neutralizing epitope) of the full-length gB protein. Several antigenic domains (AD) of gB have been described (e.g., AD-1, AD-2, AD-3, AD-4, AD-5). See, e.g., Chandramouli et al. 2015 Nat. Comm. 6 (8176): 1-12 and WO 2016/092460 (corresponding to US 2018/0265551). In certain aspects of the invention, the immunogenic fragment described herein comprises an antigenic domain selected from the group consisting of AD-1, AD-2, AD-3, AD-4, AD-5, and a combination thereof. An “immunogenic fragment” of a modified gB protein still comprises all of the modifications made to, or present in, the corresponding portion of the full length, referenced modified gB protein. Due to art-recognized sequence manipulation techniques, it is common to remove up to ten (10) consecutive amino acids at either or both of the N- and C-terminal of a sequence for, for example, recombinant ligation of two heterologous sequences (e.g., to expose a residue at the C-terminus that is sufficient for His-tag ligation). See, e.g., CHI et al. WO 2021/260510. And because certain HCMV gB domain sequences are not consecutive (e.g., Domains II, III, and IV); it may not be clear to specify that the Domain's amino acid residues are all “consecutive”. To that end, where it is specified that the HCMV gB comprises functional Domains I-V; that does not mean that full length Domains I-V sequences need to be present. An HCMV gB herein that comprises a functional Domain I comprises at least 190, 191, 192, 193, 193, 195, 196, 197, 198, 199, 200, 201, 202, 203, 204, 205, 206, 207, 208, 209, or 210 Domain I amino acids. An HCMV gB herein that comprises a functional Domain II comprises at least 83, 84, 85, 86, 87, 88, 89, 90, 91, 92, 93, 94, 95, 96, 97, 98, 99, 100, 101, 102, or 103 Domain II amino acids. An HCMV gB herein that comprises a functional Domain III comprises at least 76, 77, 78, 79, 80, 81, 82, 83, 84, 85, 86, 87, 88, 89, 90, 91, 92, 93, 94, 95, or 96 Domain III amino acids. An HCMV gB herein that comprises a functional Domain IV comprises at least 87, 88, 89, 90, 91, 92, 93, 94, 95, 96, 97, 98, 99, 100, 101, 102, 103, 104, 105, 106, or 107 Domain IV amino acids. An HCMV gB herein that comprises a functional Domain V comprises at least 30, 31, 32, 33, 34, 35, 36, 37, 38, 39, 40, 41, 42, 43, 44, 45, 46, 47, 48, 49, or 50 Domain V amino acids. In certain embodiments it may be specified that the HCMV gB Domain V amino acid sequence comprises at least 30, 31, 32, 33, 34, 35, 36, 37, 38, 39, 40, 41, 42, 43, 44, 45, 46, 47, 48, 49, or 50 consecutive amino acids from residues 649-698 (inclusive of endpoints) of SEQ ID NO: 1 (i.e., the Domain V consecutive amino acids have 100% identity to a subsequence of (or the full length of) residues 649-698 (inclusive of endpoints) of SEQ ID NO: 1). In a further embodiment it may be specified that the HCMV gB has an amino acid sequence that comprises residues corresponding to 89-648 of SEQ ID NO: 1 (i.e., full length Domains I-IV) and a Domain V amino acid sequence that is at least 30 total residues in length (such as a Domain V amino acid sequence that comprises at least 30, 31, 32, 33, 34, 35, 36, 37, 38, 39, 40, 41, 42, 43, 44, 45, 46, 47, 48, 49, or 50 consecutive amino acids from residues 649-698 (inclusive of endpoints) of SEQ ID NO: 1).

While their structure is distinct from non-modified polypeptides, the modified gB proteins of the present invention maintain immunogenic properties or epitope(s), so it is a further object of the present invention to utilize the modified polypeptides and modified fragments thereof in polypeptide/antibody interactions. The invention therefore provides antibodies which recognise a modified HCMV gB protein as described herein and, further, an antibody that is specific for such modified HCMV gB protein. The antibodies of the present invention may be a monoclonal antibody, polyclonal antibody, multispecific antibody (e.g., bispecific antibodies), labelled antibody, or antibody fragment so long as they exhibit the desired antigen-binding activity. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

Alternatively, the modified HCMV gB proteins herein may be used to identify antibodies using in vitro selection methods, such as phage display using diverse antibody libraries. The invention also provides a method for raising antibodies using a modified gB protein of the invention. An antibody or antibody fragment of the invention may be human or humanised. The antibodies of the invention may be used in a diagnostic assay and may be labelled directly or indirectly. In some embodiments, the antibodies of the invention may be used in therapy, for example in the treatment HCMV infection and may be in the form of neutralizing antibodies, which can inhibit or neutralize a gB protein's biological activity.

Attachment of a glycan to a gB protein creates a physical barrier (as well as a more hydrophilic surface) to reduce aggregation/adhesion via the hydrophobic surface. Glycosylation sites can be introduced into desired locations by suitable modification of amino acid sequences of the gB protein. See WO 2016/092460 (corresponding to US 2018/0265551). Preferably, N-linked glycosylation sites, comprising the N-X-S/T/C motif, are introduced. Preferably, the motif is N-X-S/T. Preferably, X is not proline. Similarly, sites for O-linked glycosylation can also be added. In O-linked glycosylation, the carbohydrate moiety is linked to the hydroxyl oxygen of serine and threonine. In addition, O-linked glycosylation also occurs at tyrosine, 5-hydroxylysine, and 4-hydroxyproline.

Further modifications not described herein but known to the art (specifically known to the art regarding HCMV gB proteins), as well as combinations of modifications described herein, may be performed. The resulting modifications can be analyzed, e.g., by scanning electron microscope (SEM), computer modeling, sedimentation (such as analytical ultracentrifugation (AUC)), chromatography etc., to assess the production of monomeric trimer. For example, size exclusion chromatography (SEC), such as size exclusion chromatography based on UV (SEC-UV) may be used. Alternatively, the sample can be treated with a cross-linking agent, so as to form covalent bonds between two proteins. After cross-linking, loading the sample on a gel in denaturing conditions, such as SDS-PAGE, and staining the gel for the presence of proteins, for example with Coomassie blue or silver nitrate, will display aggregates, if any, which are separated according to their molecular weight. See WO 2016/092460 (corresponding to US 2018/0265551).

A modification of HCMV gB protein residue C246 of SEQ ID NO:1 (or at a corresponding position in other HCMV gB proteins) may be introduced. Such modification can be, e.g., C246S, C246A, or C246G (or corresponding residues). It appears that C246 is an unpaired cysteine and modifying this unpaired cysteine can reduce the undesired formation of inter-molecular disulfide bonds. There is another potential unpaired cysteine at the C-terminal region (residue 779 of SEQ ID NO:1). If present, this cysteine (or corresponding cysteines in other HCMV gB proteins) may also be modified. See WO 2016/092460 (corresponding to US 2018/0265551).

Optionally, to facilitate expression and recovery, the HCMV gB protein (or immunogenic fragment thereof) may include a signal peptide at the N-terminus. A signal peptide can be selected from among numerous signal peptides known in the art, and is typically chosen to facilitate production and processing in a system selected for recombinant expression of the HCMV gB protein (or immunogenic fragment thereof). In general, signal peptides are 5-30 amino acids long, and are typically present at the N-terminus of a newly synthesized protein. The core of the signal peptide generally contains a long stretch of hydrophobic amino acids that has a tendency to form a single alpha-helix. In addition, many signal peptides begin with a short hydrophilic (usually positively charged) stretch of amino acids, which may help to enforce proper topology of the polypeptide during translocation. At the end of the signal peptide (C-terminus), there is typically a stretch of hydrophilic amino acids that is recognized and cleaved by signal peptidase. Signal peptidase may cleave either during or after completion of translocation to generate a free signal peptide and a mature protein.

In certain embodiments, the signal peptide included in the modified HCMV gB peptide is the signal peptide naturally present in the native HCMV gB protein, where “native” means naturally occurring or as found in nature. For Merlin and AD169 strains, the signal peptide is located at residues 1-22 (i.e., 1-22 of SEQ ID NO:1). Signal peptide from other HCMV strains can be identified by sequence alignment. Alternatively, the signal peptide may be a heterologous sequence in that the sequence arises from a protein distinct from gB. Exemplary signal peptides suitable for use in the context of the HCMV gB protein (or an immunogenic fragment thereof) described herein include signal peptides of tissue plasminogen activator (tPA), Herpes Simplex Virus (HSV) gD protein, human endostatin, HIV gp120, CD33, human Her2Neu, gp67, or Epstein Barr Virus (EBV) gp350. The signal peptide can be non-native and may comprise modifications, such as substitutions, insertions, or deletions of amino acids. In particular, modifications can be introduced at C-terminal part of the signal peptide.

Optionally, the modified HCMV gB proteins (or immunogenic fragment thereof) of the invention can include the addition of an amino acid sequence that constitutes a tag, which can facilitate detection (e.g. an epitope tag for detection by monoclonal antibodies) and/or purification (e.g. a polyhistidine-tag to allow purification on a nickel-chelating resin) of the proteins. Examples of affinity-purification tags include, e.g., 6×His tag (hexahistidine, binds to metal ion), maltose-binding protein (MBP) (binds to amylose), glutathione-S-transferase (GST) (binds to glutathione), FLAG tag (Asp-Tyr-Lys-Asp-Asp-Asp-Asp-Lys (SEQ ID NO: 7), binds to an anti-flag antibody), Strep tag (Ala-Trp-Arg-His-Pro-Gln-Phe-Gly-Gly (SEQ ID NO: 8), or Trp-Ser-His-Pro-Gln-Phe-Glu-Lys (SEQ ID NO: 9), or Trp-Ser-His-Pro-Gln-Phe-Glu-Lys-Gly-Gly-Gly-Ser-Gly-Gly-Gly-Ser-Gly-Gly-Gly-Ser-Trp-Ser-His-Pro-Gln-Phe-Glu-Lys (SEQ ID NO:10) binds to streptavidin or a derivative thereof), HA tag, MYC tag, or combination thereof.

In a certain embodiment, cleavable linkers may be used. This allows for the tag to be separated from the purified complex, for example by the addition of an agent capable of cleaving the linker. A number of different cleavable linkers are known to those of skill in the art. Such linkers may be cleaved for example, by irradiation of a photolabile bond or acid-catalyzed hydrolysis. There are also polypeptide linkers which incorporate a protease recognition site and which can be cleaved by the addition of a suitable protease enzyme including, for example, a Tobacco Etch Virus (TEV) cleavage linker (or cleavage site). It may be more desirable to express HCMV gB (or immunogenic fragment thereof) without an exogenous tag sequence (i.e., without a 6×His tag and TEV cleavage site), for example, for clinical safety or efficacy reasons.

When a host cell herein is cultured under suitable conditions, the nucleic acid can express a modified HCMV gB protein (or an immunogenic fragment thereof) as described herein. The modified HCMV gB protein may then be secreted from the host cell. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof.

For modified HCMV gB proteins that comprise a glycosylation site, the host cell should be one that has enzymes that mediate glycosylation. Bacterial hosts are generally not suitable for such modified proteins, unless the host cell is modified to introduce glycosylation enzymes; instead, a eukaryotic host, such as insect cell, avian cell, or mammalian cell should be used.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA. For example, for expression in insect cells a suitable baculovirus expression vector, such as pFastBac (Invitrogen), is used to produce recombinant baculovirus particles. The baculovirus particles are amplified and used to infect insect cells to express recombinant protein. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)).

Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Suitable avian cells include, for example, chicken embryonic stem cells (e.g., EBx cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

The host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, NY; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, FL. In bacterial systems, a number of expression vectors can be selected depending upon the use intended for the expressed product. For example, when large quantities of a polypeptide or fragments thereof are needed for the production of antibodies, vectors which direct high level expression of proteins that are readily purified are favorably employed. Such vectors include, but are not limited to, multifunctional E. coli cloning and expression vectors such as BLUESCRIPT (Stratagene), in which the coding sequence of interest, e.g., a polynucleotide of the invention as described above, can be ligated into the vector in-frame with sequences for the amino-terminal translation initiating methionine and the subsequent seven residues of beta-galactosidase producing a catalytically active beta galactosidase fusion protein; pIN vectors (Van Heeke & Schuster (1989) J Biol Chem 264:5503-5509); pET vectors (Novagen, Madison WI), in which the amino-terminal methionine is ligated in frame with a histidine tag; and the like. Similarly, in yeast, such as Saccharomyces cerevisiae, a number of vectors containing constitutive or inducible promoters such as alpha factor, alcohol oxidase and PGH can be used for production of the desired expression products. For reviews, see Berger, Ausubel, and, e.g., Grant et al. (1987; Methods in Enzymology 153:516-544). In mammalian host cells, a number of expression systems, including both plasmids and viral-based systems, can be utilized.

A host cell is optionally chosen for its ability to modulate the expression of the inserted sequences or to process the expressed protein in the desired fashion, including, but not limited to, glycosylation, acetylation, carboxylation, phosphorylation, lipidation, and acylation. Post-translational processing for example, which cleaves a precursor form into a mature form of the protein (for example, by a furin protease) is optionally performed in the context of the host cell. Different host cells such as 3T3, COS, CHO, HeLa, HEK, BHK, MDCK, 293, WI38, etc. have specific cellular machinery and characteristic mechanisms for such post-translational activities and can be chosen to ensure the correct modification and processing of the introduced, foreign protein. For long-term, high-yield production of modified gB protein encoded by the nucleic acids disclosed herein, stable expression systems are typically used. For example, cell lines which stably express a modified HCMV gB protein of the invention are obtained by introducing into the host cell expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells which successfully express the introduced sequences. For example, resistant groups or colonies of stably transformed cells can be proliferated using tissue culture techniques appropriate to the cell type. Host cells transformed with a nucleic acid encoding a modified HCMV gB protein are optionally cultured under conditions suitable for the expression and recovery of the encoded protein from cell culture.

Following transduction of a suitable host cell line and growth of the host cells to an appropriate cell density, the selected promoter is induced by appropriate means (e.g., temperature shift or chemical induction) and cells are cultured for an additional period. The secreted polypeptide product is then recovered from the culture medium and purified. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption. Modified gB proteins can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

Preferably, the host cells are mammalian cells (e.g., human, non-human primate, horse, cow, sheep, dog, cat, and rodent (e.g., hamster). Suitable mammalian cells include, for example, Chinese hamster ovary (CHO) cells, human embryonic kidney cells (HEK-293 cells, NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like.

In certain embodiments, the host cell is a CHO cell. In certain embodiments, the nucleic acid encoding the modified HCMV gB protein (or immunogenic fragment thereof) described herein is stably integrated into the genome of the CHO cell.

Various CHO cell lines are also available from European Collection of Cell Cultures (ECACC), or American Type Culture Collection (ATCC), such as CHO cell lines hCBE11 (ATCC PTA-3357™), E77.4 (ATCC PTA-3765™), hLT-B: R-hG1 CHO #14 (ATCC CRL-11965™), MOR-CHO-MORAb-003-RCB (ATCC PTA-7552™), AQ.C2 clone 11B (ATCC PTA-3274™), AQ.C2 clone 11B (ATCC PTA-3274™), hsAQC2 in CHO-DG44 (ATCC PTA-3356™), xrs5 (ATCC CRL-2348™), CHO-K1 (ATCC CCL-61™), Lec1 [originally named Pro-5WgaRI3C] (ATCC CRL-1735™), Pro-5 (ATCC CRL-1781™), ACY1-E (ATCC 65421™), ACY1-E (ATCC 65420™), pgsE-606 (ATCC CRL-2246™), CHO-CD36 (ATCC CRL-2092™), pgsC-605 (ATCC CRL-2245™), MC2/3 (ATCC CRL-2143™), CHO-ICAM-1 (ATCC CRL-2093™), and pgsB-618 (ATCC CRL-2241™). Any one of these CHO cell lines may be used.

Other commercially available CHO cell lines include, e.g., FREESTYLE™ CHO-S Cells and FLP-IN™-CHO Cell Line from Life Technologies.

Methods for expressing recombinant proteins in CHO cells in general have been disclosed. See, e.g., in U.S. Pat. Nos. 4,816,567 and 5,981,214.

In certain embodiments, the recombinant nucleic acids are codon optimized or codon-pair optimized for expression in a selected prokaryotic or eukaryotic host cell.

To facilitate replication and expression, the nucleic acids can be incorporated into a vector, such as a prokaryotic or a eukaryotic expression vector. Exemplary vectors include plasmids that are able to replicate autonomously or to be replicated in a host cell. Typical expression vectors contain suitable promoters, enhancers, and terminators that are useful for regulation of the expression of the coding sequence(s) in the expression construct. The vectors may also comprise selection markers to provide a phenotypic trait for selection of transformed host cells (such as conferring resistance to antibiotics such as ampicillin or neomycin). Nucleic acid or vector modification may be undertaken in a manner known by the art, see e.g., WO 2012/049317 (corresponding to US 2013/0216613) and WO 2016/092460 (corresponding to US 2018/0265551). For example, the nucleic acids that encode a modified HCMV gB protein as described herein are cloned into a vector suitable for introduction into mammalian cells (e.g., CHO cells). In this exemplary embodiment, the polynucleotide sequence that encodes the modified HCMV gB protein is introduced into the pMax vector developed by Amaxa. The polypeptide is expressed under a constitutive promoter, the immediate early CMV promoter. Selection of the stably transfected cells expressing the polypeptide is made based on the ability of the transfected cells to grow in the presence of kanamycin. Cells that have successfully integrated the pMax are able to grow in the presence of kanamycin, because the pMax vector expresses a kanamycin resistance gene. Selected cells can be clonally expanded and characterized for expression of the modified HCMV gB proteins. Alternatively, the polynucleotide sequences that encode the modified gB proteins of the invention may be introduced into the pTT5 vector developed by NRC, which expresses an ampicillin resistance gene.

Following transfection and induction of expression (according to the selected promoter and/or enhancers or other regulatory elements), the expressed polypeptides are recovered (e.g., purified, isolated, or enriched). To facilitate purification, the modified HCMV gB protein(s) may include a C-terminal polyhistidine tag (e.g., 6×His tag) and, optionally, further include a cleavage site (e.g., a TEV cleavage site).

The term “purification” or “purifying” refers to the process of removing components from a composition or host cell or culture, the presence of which is not desired. Purification is a relative term and does not require that all traces of the undesirable component be removed from the composition. In the context of vaccine production, purification includes such processes as centrifugation, dialyzation, ion-exchange chromatography, and size-exclusion chromatography, affinity-purification or precipitation. Thus, the term “purified” does not require absolute purity; rather, it is intended as a relative term. A preparation of substantially pure nucleic acid or protein can be purified such that the desired nucleic acid, or protein, represents at least 50% of the total nucleic acid content of the preparation. In certain embodiments, a substantially pure nucleic acid, or protein, will represent at least 60%, at least 70%, at least 80%, at least 85%, at least 90%, or at least 95% or more of the total nucleic acid or protein content of the preparation. Immunogenic molecules or antigens or antibodies which have not been subjected to any purification steps (i.e., the molecule as it is found in nature) are not suitable for pharmaceutical (e.g., vaccine) use.

In the sense of the present invention, a “purified” or an “isolated” biological component (such as a nucleic acid molecule, or protein) has been substantially separated or purified away from other biological components in the cell of the organism in which the component naturally occurs or was produced, such as, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. A molecule that is “purified” or “isolated” is not binding to a heterologous molecule such as an antibody or antibody fragment, or to a helper molecule like the inhibitor in Liu et al. 2021 Sci. Adv. 7: eabf3178 and CHI et al. WO2021260510.

In certain embodiments, the modified HCMV gB protein (or immunogenic fragment thereof) described herein is purified or isolated. The modified gB protein (or immunogenic fragment thereof) can be purified using any suitable methods, such as HPLC, various types of chromatography (such as hydrophobic interaction, ion exchange, affinity, chelating, and size exclusion), electrophoresis, density gradient centrifugation, solvent extraction, or the like. For example, methods for purifying HCMV gB protein by immunoaffinity chromatography has been disclosed. Ruiz-Arguello et al., J. Gen. Virol., 85:3677-3687 (2004). As appropriate, the modified gB protein (or immunogenic fragment thereof) may be further purified, as required, so as to remove substantially any proteins which are also secreted in the medium or result from lysis of host cells, so as to provide a product which is at least substantially free of host debris, e.g., proteins, lipids and polysaccharides. See, e.g., those set forth in Sandana (1997) Bioseparation of Proteins, Academic Press, Inc.; and Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ. If desired, the modified gB protein (or immunogenic fragment thereof) can include a “tag” that facilitates purification, as described above.

Exemplary procedures sufficient to guide one of ordinary skill in the art through the production of modified HCMV gB proteins and nucleic acids encoding them can be found in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

“Recombinant” as used herein to describe a polynucleotide means a polynucleotide of genomic, cDNA, RNA (including mRNA) semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. The term “recombinant” may also be used herein to clarify that the referenced molecule (e.g., a nucleic acid or amino acid) does not naturally exist at that position within the sequence (e.g., it exists there as the result of mutation). Further, “recombinant” may be used herein to refer to specific interactions (e.g., disulfide bonds) which do not naturally exist and are now present, e.g., as a result of mutation (such as point mutations causing amino acid substitutions). Such recombinant disulfide bonds include herein:
a disulfide bond between a recombinant Cysteine at residue number 269 and a recombinant Cysteine at residue number 656 (numbered according to SEQ ID NO: 1) [e.g., S269C-L656C];
a disulfide bond between a recombinant Cysteine at residue number 524 and a recombinant Cysteine at residue 684 (numbered according to SEQ ID NO:1) [e.g., N524C-M684C]; and
a disulfide bond between a recombinant Cysteine at residue number 217 and a recombinant Cysteine at residue 587 (numbered according to SEQ ID NO: 1) [e.g., D217C-S587C].

In certain embodiments it may be specified that the HCMV gB comprises at least one, at least two, at least three, at least four, at least five, or at least six recombinant Cysteine (C) residues. In a further embodiment it may be specified that the HCMV gB comprises a recombinant Cysteine (C) at the residue corresponding to 100, 156, 217, 239, 251, 267, 269, 524, 587, 614, 656, 684, or combinations thereof, of SEQ ID NO: 1. In yet another embodiment it may be specified that the HCMV gB comprises an amino acid sequence that consists of (i.e., has no more than) one, two, three, four, five, or six recombinant Cysteine (C) residues. It may be further specified that the HCMV gB comprises an amino acid sequence that consists of (i.e., has no more than) one, two, three, four, five, or six recombinant Cysteines (C) at the residues corresponding to 100, 156, 217, 239, 251, 267, 269, 524, 587, 614, 656, and/or 684 of SEQ ID NO: 1.

A “heterologous sequence” with regard to a HCMV gB protein refers to an amino acid or nucleotide sequence that is not found in naturally occurring HCMV gB protein, or in a nucleic acid encoding an HCMV gB protein, respectively. When a nucleic acid molecule is operably linked to another polynucleotide that it is not associated with in nature, the nucleic acid molecule may be referred to as “heterologous” (i.e., the nucleic acid molecule is heterologous to at least the polynucleotide). Similarly, when a polypeptide is in contact with or in a complex with another protein that it is not associated with in nature, the polypeptide may be referred to as “heterologous” (i.e., the polypeptide is heterologous to the protein). Further, when a host cell comprises a nucleic acid molecule or polypeptide that it does not naturally comprise, the nucleic acid molecule and polypeptide may be referred to as “heterologous” (i.e., the nucleic acid molecule is heterologous to the host cell and the polypeptide is heterologous to the host cell).

It will be understood by those of skill in the art, that the similarity between modified HCMV gB proteins (or polynucleotide sequences), as for protein (or nucleotide sequences) in general, can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid (or polynucleotide) sequences, the more similar are the higher order structures resulting from folding and assembly. Variants of HCMV gB proteins and polynucleotide sequences (e.g., those originating from different HCMV strains) can have one or a small number of amino acid deletions, additions or substitutions as compared to another, but will nonetheless share a very high percentage of their amino acid, and generally their polynucleotide sequence. Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Sequence identity between polypeptide sequences is preferably determined by pairwise alignment algorithm using the Needleman-Wunsch global alignment algorithm (Needleman and Wunsch, A General Method Applicable to the Search for Similarities in the Amino Acid Sequence of Two Proteins, 1970 J. Mol. Biol. 48 (3): 443-453), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice et al., EMBOSS: The European Molecular Biology Open Software Suite, 2000 Trends Genetics 16:276-277). Sequence identity should be calculated over the entire length of the polypeptide sequence of the invention.

A “subject” is a living multi-cellular vertebrate organism. In the context of this disclosure, the subject can be an experimental subject, such as a non-human mammal, e.g., a mouse, a cotton rat, or a non-human primate. Alternatively, the subject can be a human subject.

An “antigen” is a compound, composition, or substance that can stimulate an immune response by producing antibodies and/or a T cell response in a mammal, including compositions that are injected, absorbed or otherwise introduced into a mammal. The term “antigen” includes all related antigenic epitopes. The term “epitope” or “antigenic determinant” refers to a site on an antigen to which B and/or T cells respond. The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”), such as an HCMV antigen. If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

Typically, the immune response elicits an immune response characterized by the production of Th1 type cytokines, e.g. a Th1-type immune response. A “Th1” type immune response is characterized CD4+ T helper cells that produce IL-2 and IFN-γ. In contrast, a “Th2” type immune response is characterized by CD4+ helper cells that produce IL-4, IL-5, and IL-13.

An “effective amount” means an amount sufficient to cause the referenced effect or outcome. An “effective amount” can be determined empirically and in a routine manner using known techniques in relation to the stated purpose. An “immunologically effective amount” is a quantity of a composition (typically, an immunogenic composition) sufficient to elicit an immune response in a subject (either in a single dose or in a series). Commonly, the desired result is the production of an antigen (e.g., pathogen)-specific immune response that is capable of or contributes to protecting the subject against the pathogen, such as HCMV. However, to obtain a protective immune response against a pathogen can require multiple administrations of the immunogenic composition. Thus, in the context of this disclosure, the term immunologically effective amount encompasses a fractional dose that contributes in combination with previous or subsequent administrations to attaining a protective immune response.

Also provided are immunogenic compositions or pharmaceutical compositions, such as vaccines, that comprise a modified HCMV gB protein and a pharmaceutically acceptable diluent, carrier or excipient. An “immunogenic composition” is a pharmaceutical composition of matter suitable for administration to a human or non-human mammalian subject that is capable of eliciting a specific immune response, e.g., against a pathogen, such as HCMV. As such, an immunogenic composition includes one or more antigens (for example, polypeptide antigens or an RNA or DNA that encodes a polypeptide antigen) or antigenic epitopes, such as for instance, the modified HCMV gB protein of the inventions. An immunogenic composition can also include one or more additional components capable of enhancing an immune response, such as an excipient, carrier, and/or adjuvant.

In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., HCMV) following exposure of the subject to the pathogen. In the context of this disclosure, the term immunogenic composition will be understood to encompass compositions that are intended for administration to a subject or population of subjects for the purpose of eliciting a protective or palliative immune response against HCMV.

The immunogenic compositions according to the invention are not limited to compositions consisting of modified HCMV gB proteins. The present invention also contemplates immunogenic compositions, such as vaccines, comprising the modified HCMV gB protein of the invention and at least one or more HCMV antigens. Such one or more HCMV antigens may be pp65, IE1, gO, gL, gH, pUL128, pUL130, pUL131, or any combination thereof, or any complex-forming fragment thereof. As an example, a composition comprising a modified HCMV gB protein as described herein and pp65 is contemplated. Further, a composition comprising a modified protein as described herein and IE1 is contemplated. Further, a composition comprising a modified HCMV gB protein as described herein and gL, gH, pUL128, pUL130, and pUL131 is contemplated. It is contemplated that the compositions herein may comprise one or more nucleic acids (e.g., an RNA) that together encode a modified HCMV gB protein as described herein and one or more of a gO, gL, gH, pUL128, pUL130, pUL131, pp65, or IE1 protein (e.g., encode a HCMV gO, gL, gH, pUL128, pUL130, pUL131, pp65, or IE1 protein). By “complex-forming fragment” it is meant any part or portion of the polypeptide that retains the ability to form a complex (e.g., the pentameric complex, gH/gL dimer, and gH/gL/gO trimer) with other polypeptides of the complex. As used herein, a “complex-forming fragment” of a modified protein comprises the one or more modified amino acid residues (i.e., the fragment of a modified protein comprises the modification(s)). Where the composition comprises gL, gH, pUL128, pUL130, or pUL131 (five proteins that together form what is called a “pentamer complex”), a “pentamer-forming” fragment of gL, gH, pUL128, pUL130, or pUL131 may be utilized (see WO 2014/005959 (corresponding to U.S. Pat. No. 9,683,022); WO2016/116904 (corresponding to U.S. Pat. No. 10,167,321); and WO 2018/193307). Wherein the composition comprises gH (e.g., HCMV gH), the gH may be a complex-forming gH fragment that lacks a transmembrane domain (see WO 2014/005959 (corresponding to U.S. Pat. No. 9,683,022). Wherein the composition comprises gL (e.g., HCMV gL), the gL may be a modified gL protein having a modification within a protease recognition site, e.g., a modification within a protease recognition site that reduces protease cleavage of gL (see WO2016/116904 (corresponding to U.S. Pat. No. 10,167,321)). Wherein the composition comprises one or more HCMV gL, gH, pUL128, pUL130, or pUL131 proteins; such one or more HCMV proteins may comprise one or more stabilizing modification as previously disclosed within WO 2018/193307.

Numerous pharmaceutically acceptable diluents and carriers and/or pharmaceutically acceptable excipients are known in the art and are described, e.g., in Remington's Pharmaceutical Sciences, by E. W. Martin, Mack Publishing Co., Easton, PA, 15th Edition (1975). The adjective “pharmaceutically acceptable” indicates that the diluent, or carrier, or excipient, is suitable for administration to a subject (e.g., a human or non-human mammalian subject). In general, the nature of the diluent, carrier and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid carriers can be used, including for example, pharmaceutical grades of trehalose, mannitol, lactose, starch or magnesium stearate. Suitable carriers are typically large, slowly metabolized macromolecules such as proteins (e.g., nanoparticles), polysaccharides, polylactic acids, polyglycolic acids, polymeric amino acids, amino acid copolymers, lipid aggregates (such as oil droplets or liposomes), and inactive virus particles. Such carriers are well known in the art.

Accordingly, suitable excipients and carriers can be selected by those of skill in the art to produce a formulation suitable for delivery to a subject by a selected route of administration. Excipients include, without limitation: glycerol, polyethylene glycol (PEG), glass forming polyols (such as, sorbitol, trehalose) N-lauroylsarcosine (e.g., sodium salt), L-proline, non detergent sulfobetaine, guanidine hydrochloride, urea, trimethylamine oxide, KCl, Ca2+, Mg2+, Mn2+, Zn2+ (and other divalent cation related salts), dithiothreitol (DTT), dithioerytrol, β-mercaptoethanol, detergents (including, e.g., TWEEN 80, TWEEN 20, TRITON X-100, NP-40, EMPIGEN BB, Octylglucoside, Lauroyl maltoside, ZWITTERGENT 3-08, ZWITTERGENT 3-10, ZWITTERGENT 3-12, ZWITTERGENT 3-14, ZWITTERGENT 3-16, CHAPS, sodium deoxycholate, sodium dodecyl sulphate, and cetyltrimethylammonium bromide.

In certain embodiments, the antigen(s) may be conjugated to a bacterial toxoid, such as a toxoid from diphtheria, tetanus, cholera, H. pylori, or another pathogen.

In certain examples, the modified HCMV gB protein is operably linked to a carrier wherein the carrier is a nanoparticle (e.g., a virus like particle (VLP) or self-assembling protein nanoparticle such as a lumazine synthase nanoparticle, ferritin nanoparticle, or an aldolase-based nanoparticle). Pharmaceutically acceptable nanoparticles as carriers, as well as methods of using them to present an antigen, are known and include lumazine synthase, ferritin, or aldolase-based nanoparticles (or nanocages) or nanoparticles derived therefrom (see Lopez-Sagaseta et al., 2016 Comput Struct Biotechnol J, 14:58-68; Nguyen and Tolia 2021 npj Vaccines 6 (70): 1-11; WO 2005/121330; WO 2013/044203; WO 2016/037154; and Bruun et al. 2018 ACS Nano 12 (9): 8855-8866). Such nanoparticles may be “self-assembling” (see WO 2015/048149). In the context of nanoparticles (or nanocages) as carriers, operable linkage of antigens onto a nanoparticle can be achieved through a variety of techniques including spontaneous isopeptide bond formation, chemical conjugation, genetic fusion, or bio-orthogonal chemistry with unnatural amino acids (see Bruun et al. 2018 ACS Nano 12 (9): 8855-8866 at 8855 and references therein). See, e.g., WO2015/156870 (PCT/US2015/011534, DENG Z.), describing nanoparticle-polypeptide conjugates linked through an isopeptide bond (see also Bruun et al. 2018 ACS Nano 12 (9): 8855-8866 describing operable linkage to aldolase nanoparticles through isopeptide bond (“SpyTag-SpyCatcher”)). Nanoparticles operably linked to an antigen in pre-fusion conformation has been demonstrated for RSV F proteins (U.S. Pat. No. 9,738,689).

The pharmaceutical formulations are conventionally administered parenterally, e.g., by injection, either subcutaneously, intraperitoneally, transdermally, or intramuscularly. Some embodiments will be administered through an intra-mucosal route such as intra-orally, intra-nasally, intra-vaginally, and intra-rectally. Additional formulations suitable for other modes of administration include oral and pulmonary formulations, suppositories, and transdermal applications. Oral formulations may be preferred for certain viral proteins. Dosage treatment may be a single dose schedule or a multiple dose schedule. The immunogenic composition may be administered in conjunction with other immunoregulatory agents. Any suitable route of administration can be used. For example, a composition can be administered intramuscularly, intraperitoneally, subcutaneously, or transdermally. Compositions can be administered according to any suitable schedule.

Typically, the pharmaceutical formulations are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

In certain examples, the immunogenic composition also includes an adjuvant. Suitable adjuvants for use in immunogenic compositions containing modified HCMV gB proteins of the invention are adjuvants that in combination with said polypeptides disclosed herein are safe and having acceptable reactogenicity when administered to a subject.

An “adjuvant” is an agent that enhances the production of an immune response in a non-specific manner. Common adjuvants include suspensions of minerals (alum, aluminum hydroxide, aluminum phosphate) onto which antigen may be adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll Receptor agonists (particularly, TLR2, TLR4, TLR7/8 and TLR9 agonists), and various combinations of such components. In certain embodiment, the adjuvant is a TLR7 agonist, such as imidazoquinoline or imiquimod. In certain embodiment, the adjuvant is an aluminum salt, such as aluminum hydroxide, aluminum phosphate, aluminum sulfate. The adjuvants described herein can be used singularly or in any combination, such as alum/TLR7 (also called AS37).

Suitable adjuvants for use in combination with the modified gB proteins of the invention are saponins. Accordingly, immunogenic compositions of the invention may comprise the saponin fraction QS21 (WO8809336A1; U.S. Pat. No. 5,057,540A). QS21 is well known in the art as a natural saponin derived from the bark of Quillaja saponaria Molina, which induces CD8+ cytotoxic T cells (CTLs), Th1 cells and a predominant IgG2a antibody response. For the avoidance of doubt reference to QS21 includes OPT-821. In a suitable form of the present invention, the immunogenic compositions of the invention comprise QS21 in substantially pure form, that is to say, the QS21 is at least 80%, at least 85%, at least 90% pure, for example at least 95% pure, or at least 98% pure. A suitable QS-21 fraction is as described in WO 2019/10692.

The immunogenic compositions comprising the modified HCMV gB proteins of the invention may comprise a QS21 fraction and a sterol, cholesterol in particular. Such compositions show a decreased reactogenicity when compared to compositions in which the sterol is absent, while the adjuvant effect is maintained. Reactogenicity studies may be assessed according to the methods disclosed in WO 96/33739. Suitably the sterol is associated to the saponin adjuvant as described in WO 96/33739. In a particular embodiment, the cholesterol is present in excess to that of QS21, for example, the ratio of QS21:sterol will typically be in the order of 1:100 to 1:1 (w/w), suitably between 1:10 to 1:1 (w/w), and preferably 1:5 to 1:1 (w/w). In particular, the ratio of QS21:sterol being at least 1:2 (w/w). In a particular embodiment, the ratio of QS21:sterol is 1:5 (w/w). Suitable sterols include β-sitosterol, stigmasterol, ergosterol, ergocalciferol and cholesterol. In one particular embodiment, the compositions of the invention comprise cholesterol as sterol. These sterols are well known in the art, for example cholesterol is disclosed in the Merck Index, 11th Edn, page 341, as a naturally occurring sterol found in animal fat. Accordingly, in a specific embodiment, immunogenic compositions comprising the modified gB proteins of the invention comprise QS21 in its less reactogenic composition where it is quenched with a sterol, such as cholesterol for example. Several particular forms of less reactogenic compositions wherein QS21 is quenched with a cholesterol exist. In a specific embodiment, the saponin/sterol is in the form of a liposome structure (WO 96/337391). Thus, for example, modified HCMV gB proteins of the invention can suitably be employed in immunogenic compositions with an adjuvant comprising a combination of a QS21 fraction and cholesterol.

The term “liposome(s)” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids, which are capable of forming liposomes include all substances having fatty or fat-like properties. Lipids which can make up the lipids in the liposomes can be selected from the group comprising of glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids. Liposomes may suitably comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], distearoyl phosphatidylcholine [DSPC], dioleoyl phosphatidylcholine [DOPC], 1-palmitoyl, 2-oleoylphosphatidylcholine [POPC], dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phosphoserine, polyethylene glycol [PEG] phospholipid (mPEG-phospholipid, polyglycerin-phospholipid, functionalized-phospholipid, terminal activated-phospholipid). In one embodiment the liposomes comprise 1-palmitoyl-2-oleoyl-glycero-3-phosphoethanolamine. In one embodiment highly purified phosphatidylcholine is used and can be selected from the group comprising phosphatidylcholine (Egg), phosphatidylcholine hydrogenated (Egg) phosphatidylcholine (Soy) phosphatidylcholine hydrogenated (Soy). In a further embodiment the liposomes comprise phosphatidylethanolamine [POPE] or a derivative thereof. Liposome size may vary from 30 nm to several μm depending on the phospholipid composition and the method used for their preparation. In particular embodiments of the invention, the liposome size will be in the range of 50 nm to 500 nm and in further embodiments 50 nm to 200 nm. Dynamic laser light scattering is a method used to measure the size of liposomes well known to those skilled in the art. Liposomes of the invention may comprise dioleoyl phosphatidylcholine [DOPC] and a sterol, in particular cholesterol. Thus, in a particular embodiment, immunogenic compositions comprising the modified HCMV gB proteins of the invention, comprise QS21 in the form of a liposome, wherein said liposome comprises dioleoyl phosphatidylcholine [DOPC] and a sterol, in particular cholesterol.

Immunogenic compositions of the invention may comprise one or more further immunostimulants. In one embodiment, immunogenic compositions comprising the modified HCMV gB proteins of the invention as described herein further comprise a lipopolysaccharide, suitably a non-toxic derivative of lipid A, particularly monophosphoryl lipid A or more particularly 3-Deacylated monophosphoryl lipid A (3D-MPL). 3D-MPL is sold under the name MPL by GlaxoSmithKline Biologicals and is referred throughout the specification as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 mm filter. Such preparations are described in WO94/21292.

In other embodiments, the lipopolysaccharide can be a beta (1.fwdarw.6) glucosamine disaccharides glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. In addition to the described immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are described as TLR-4 agonists (see WO 95/14026; WO 99/64301 and WO 00/0462; WO 01/46127).

Combinations of different adjuvants, such as those mentioned hereinabove, can also be used in compositions with modified HCMV gB proteins. For example, as already noted, the QS21 fraction can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL:QS21. Accordingly, in some embodiments, immunogenic compositions comprising modified gB proteins of the invention comprise at least QS21 and 3D-MPL.

The immunogenic compositions comprising the modified HCMV gB proteins of the invention may also be suitably formulated with an oil-in-water emulsion. The oil in water emulsion comprises a metabolisable oil (i.e. biodegradable). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also suitable. Accordingly, oil-in-water emulsions used in combination with the modified HCMV gB proteins of the invention comprise a metabolisable oil. In a particular embodiment, oil-in-water emulsions comprise squalene (for example between about 4% and 6% [v/v]). The oil-in-water emulsion may further comprise a surfactant. Oil-in-water emulsions of the invention comprise one or more surfactants. Suitable surfactants are well known to the skilled person and include, but are not limited to, polyoxyethylene sorbitan monooleate (TWEEN 80, Polysorbate 80), sorbitan triolate (SPAN 85), phosphatidylcholine (lecithin), polyoxyethylene (12) cetostearyl ether and octoxynol-9 (TRITON X-100). In a particular embodiment of the invention, oil-in-water emulsions comprise is polyoxyethylene sorbitan monooleate (TWEEN 80, Polysorbate 80). In a further embodiment, oil in water emulsions of the invention comprise polyoxyethylene sorbitan monooleate (TWEEN 80) and a further surfactant, in particular sorbitan trioleate (SPAN 85). Oil-in-water emulsions of the invention may also comprise a tocol. Tocols are well known in the art and are described in EP0382271. In particular, the tocol is α-tocopherol or a derivative thereof such as alpha-tocopherol succinate (also known as vitamin E succinate). In a particular embodiment of the invention, there is provided immunogenic compositions comprising the modified gB protein of the invention in combination with an oil-in-water emulsion comprising squalene (for example about 5% [v/v]) and α-tocopherol (for example about 5% [v/v]). In a particular embodiment, the oil-in-water emulsion comprises a metabolisable oil (e.g. squalene), a tocol (e.g. α-tocopherol) and a surfactant (e.g. polyoxyethylene sorbitan monooleate [Polysorbate 80]). In a further embodiment of the invention, oil-in-water emulsions of the invention comprise a metabolisable oil (e.g. squalene), a surfactant (e.g. polyoxyethylene sorbitan monooleate [Polysorbate 80]), and optionally a second surfactant (e.g. sorbitan trioleate [SPAN 85]). In a further embodiment of the invention, oil-in-water emulsions of the invention comprise a metabolisable oil (e.g. squalene), a polyoxyethylene alkyl ether hydrophilic non-ionic surfactant (e.g. polyoxyethylene (12) cetostearyl ether) and a hydrophobic non-ionic surfactant (e.g. polyoxyethylene sorbitan monooleate [Polysorbate 80]), or sorbitan trioleate [SPAN 85]). In some embodiments, immunogenic compositions comprise an oil-in-water emulsion comprising squalene, alpha-tocopherol, and Polysorbate 80.

Suitably, the oil-in-water comprises 11 mg metabolisable oil (such as squalene) or below, for example between 0.5-11 mg, 0.5-10 mg or 0.5-9 mg 1-10 mg, 1-11 mg, 2-10 mg, 4-8 mg, or 4.5-5.5 mg, and 5 mg emulsifying agent (such as polyoxyethylene sorbitan monooleate) or below, for example between 0.1-5 mg, 0.2-5 mg, 0.3-5 mg, 0.4-5 mg, 0.5-4 mg, 1-2 mg or 2-3 mg per dose of the vaccine. Suitably tocol (e.g. alpha-tocopherol) where present is 12 mg or below, for example between 0.5-12 mg, 10-11 mg, 1-11 mg, 2-10 mg, 4-9 mg, or 5-7 mg per human vaccine dose. By the term “vaccine human dose” is meant a dose which is in a volume suitable for human use. Generally, this is between 0.25 and 1.5 ml. In one embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml. In another embodiment, in particular when the immunogenic composition is for the pediatric population, a human dose may be less than 0.5 ml such as between 0.25 and 0.5 ml.

An immunogenic composition typically contains an immunoprotective quantity (or a fractional dose thereof) of the antigen and can be prepared by conventional techniques. Preparation of immunogenic compositions, such as vaccines, including those for administration to human subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Maryland, U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757. Typically, the amount of protein in each dose of the immunogenic composition is selected as an amount which induces an immunoprotective response without significant, adverse side effects in the typical subject. Immunoprotective in this context does not necessarily mean completely protective against infection; it means protection against symptoms or disease, especially severe disease associated with the virus. The amount of antigen can vary depending upon which specific immunogen is employed. The amount utilized in an immunogenic composition is selected based on the subject population (e.g., infant or elderly). An optimal amount for a particular composition can be ascertained by standard studies involving observation of antibody titres and other responses in subjects. Following an initial vaccination, subjects can receive a boost in about four weeks.

In some embodiments, modified HCMV gB proteins (or immunogenic fragments thereof) and/or one or more additional immunogenic protein (e.g., HCMV antigen) described herein are delivered using alphavirus replicon particles (VRP). As used herein, the term “alphavirus” has its conventional meaning in the art and includes various species such as Venezuelan equine encephalitis virus (VEE; e.g., Trinidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus.

An “alphavirus replicon particle” (VRP) or “replicon particle” is an alphavirus replicon packaged with alphavirus structural proteins.

An “alphavirus replicon” (or “replicon”) is an RNA molecule which can direct its own amplification in vivo in a target cell. The replicon encodes the polymerase(s) which catalyze RNA amplification (nsP1, nsP2, nsP3, nsP4) and contains cis RNA sequences required for replication which are recognized and utilized by the encoded polymerase(s). An alphavirus replicon typically contains the following ordered elements: 5′ viral sequences required in cis for replication, sequences which encode biologically active alphavirus nonstructural proteins (nsP1, nsP2, nsP3, nsP4), 3′ viral sequences required in cis for replication, and a polyadenylate tract. An alphavirus replicon also may contain one or more viral subgenomic “junction region” promoters directing the expression of heterologous nucleotide sequences, which may, in certain embodiments, be modified in order to increase or reduce viral transcription of the subgenomic fragment and heterologous sequence(s) to be expressed. Other control elements can be used, such as IRES or 2A sequences.

Recombinant nucleic acids that encode the modified HCMV gB proteins or immunogenic fragments described herein can be administered to induce production of the encoded modified gB proteins or immunogenic fragments and an immune response thereto.

The recombinant nucleic acid can be DNA (e.g., plasmid or viral DNA) or RNA, such as mRNA (e.g., self-amplifying mRNA), and can be monocistronic or polycistronic. Any suitable DNA or RNA can be used as the nucleic acid vector that carries the open reading frames that encode HCMV gB proteins or immunogenic fragments thereof. Suitable nucleic acid vectors have the capacity to carry and drive expression of one or more modified HCMV gB proteins or immunogenic fragments. Such nucleic acid vectors are known in the art and include, for example, plasmids, DNA obtained from DNA viruses such as vaccinia virus vectors (e.g., NYVAC, see U.S. Pat. No. 5,494,807), adenoviral vectors and poxvirus vectors (e.g., ALVAC canarypox vector, Sanofi Pasteur), and RNA obtained from suitable RNA viruses such as alphavirus. If desired, the recombinant nucleic acid molecule can be modified, e.g., contain modified nucleobases and or linkages as described further herein.

The self-amplifying RNA molecules of the invention are based on the genomic RNA of RNA viruses, but lack the genes encoding one or more structural proteins. The self-amplifying RNA molecules are capable of being translated to produce non-structural proteins of the RNA virus and modified HCMV gB proteins encoded by the self-amplifying RNA.

The self-amplifying RNA generally contains at least one or more genes selected from the group consisting of viral replicase, viral proteases, viral helicases and other nonstructural viral proteins, and also comprise 5′- and 3′-end cis-active replication sequences, and a heterologous sequence that encodes one or more desired modified HCMV gB proteins. A subgenomic promoter that directs expression of the heterologous sequence(s) can be included in the self-amplifying RNA. If desired, a heterologous sequence may be fused in frame to other coding regions in the self-amplifying RNA and/or may be under the control of an internal ribosome entry site (IRES).

Self-amplifying RNA molecules of the invention can be designed so that the self-amplifying RNA molecule cannot induce production of infectious viral particles. This can be achieved, for example, by omitting one or more viral genes encoding structural proteins that are necessary for the production of viral particles in the self-amplifying RNA. For example, when the self-amplifying RNA molecule is based on an alpha virus, such as Sindbis virus (SIN), Semliki forest virus and Venezuelan equine encephalitis virus (VEE), one or more genes encoding viral structural proteins, such as capsid and/or envelope glycoproteins, can be omitted. If desired, self-amplifying RNA molecules of the invention can be designed to induce production of infectious viral particles that are attenuated or virulent, or to produce viral particles that are capable of a single round of subsequent infection.

A self-amplifying RNA molecule can, when delivered to a vertebrate cell even without any proteins, lead to the production of multiple daughter RNAs by transcription from itself (or from an antisense copy of itself). The self-amplifying RNA can be directly translated after delivery to a cell, and this translation provides a RNA-dependent RNA polymerase which then produces transcripts from the delivered RNA. Thus, the delivered RNA leads to the production of multiple daughter RNAs. These transcripts are antisense relative to the delivered RNA and may be translated themselves to provide in situ expression of encoded HCMV protein, or may be transcribed to provide further transcripts with the same sense as the delivered RNA which are translated to provide in situ expression of the encoded HCMV protein(s).

A preferred self-amplifying RNA molecule thus encodes (i) a RNA-dependent RNA polymerase which can transcribe RNA from the self-amplifying RNA molecule and (ii) one or more modified HCMV gB proteins or immunogenic fragments thereof. The polymerase can be an alphavirus replicase e.g. comprising alphavirus non-structural proteins nsP1-nsP4.

The self-amplifying RNA molecules of the invention can contain one or more modified nucleotides and therefore have improved stability and be resistant to degradation and clearance in vivo, and other advantages. There are more than 96 naturally occurring nucleoside modifications found on mammalian RNA. See, e.g., Limbach et al., Nucleic Acids Research, 22 (12): 2183-2196 (1994). The preparation of nucleotides and modified nucleotides and nucleosides are well-known in the art, e.g. from U.S. Pat. Nos. 4,373,071, 4,458,066, 4,500,707, 4,668,777, 4,973,679, 5,047,524, 5,132,418, 5,153,319, 5,262,530, 5,700,642 all of which are incorporated herein by reference in their entirety, and many modified nucleosides and modified nucleotides are commercially available. If desired, the self-amplifying RNA molecule can contain phosphoramidate, phosphorothioate, and/or methylphosphonate linkages.

The self-amplifying RNA described herein is suitable for delivery in a variety of modalities, such as naked RNA delivery or in combination with lipids, polymers or other compounds that facilitate entry into the cells. Self-amplifying RNA molecules can be introduced into target cells or subjects using any suitable technique, e.g., by direct injection, microinjection, electroporation, lipofection, biolistics, and the like. The self-amplifying RNA molecule may also be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine amino acids), which is itself coupled to an integrin receptor-binding moiety (e.g., a cyclic peptide having the sequence Arg-Gly-Asp).

The self-amplifying RNA molecules can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

The self-amplifying RNA can be delivered as naked RNA (e.g. merely as an aqueous solution of RNA) but, to enhance entry into cells and also subsequent intercellular effects, the self-amplifying RNA is preferably administered in combination with a delivery system, such as a particulate or emulsion delivery system. A large number of delivery systems are well known to those of skill in the art. Three particularly useful delivery systems are (i) liposomes, (ii) non-toxic and biodegradable polymer microparticles, and (iii) cationic submicron oil-in-water emulsions.

The invention also provides immunogenic composition comprising the nucleic acid (e.g., self-amplifying RNA) described herein. The immunogenic composition may comprise an adjuvant, as described above. Preferred adjuvants include, e.g., an aluminum salt or an oil-in-water emulsion (such as MF59).

A nucleic acid approach for the prevention or treatment of HCMV using gB protein optionally in combination with other HCMV protein(s) was described previously, e.g., WO 2012/051211 and John et al. 2018 Vaccine 36 (12): 1689-1699.

Methods of inducing an immune response against HCMV may comprise the production of neutralizing antibodies against HCMV. In certain embodiments, the neutralizing antibodies are complement-independent. Alternatively, the induced immune response can comprise a humoral immune response, a cell-mediated immune response, or both. In some embodiments an immune response is induced against each delivered HCMV gB protein and, as relevant, other virus antigens having been delivered (e.g., pentamer complex). A cell-mediated immune response can comprise a helper T-cell (Th) response, a CD8+ cytotoxic T-cell (CTL) response, or both. In some embodiments the induced immune response comprises a humoral immune response, and the antibodies are neutralizing antibodies. Neutralizing antibodies block viral infection of cells. With regard to HCMV, the HCMV infects epithelial cells and also fibroblast cells and, in some embodiments the immune response reduces or prevents HCMV infection of both cell types. Neutralizing antibody responses can be complement-dependent or complement-independent. In some embodiments the induced neutralizing antibody response is complement-dependent. In some embodiments the induced neutralizing antibody response is cross-neutralizing; e.g., with regard to HCMV, an antibody generated against an administered composition neutralizes a HCMV virus of one or more strains in addition to the strain used in the composition.

An immune response can be stimulated by administering proteins, DNA molecules, RNA molecules (e.g., self-amplifying RNA molecules), or VRPs to an individual subject, typically a mammal, including a human. In some embodiments the immune response induced is a protective immune response, i.e., the response reduces the risk or severity of HCMV infection. Stimulating a protective immune response is particularly desirable in populations particularly at risk for HCMV infection, including solid organ transplant (SOT) patients, bone marrow transplant patients, and hematopoietic stem cell transplant (HSCT) patients. VRPs can be administered to a transplant donor pre-transplant, or a transplant recipient pre- and/or post-transplant. Because vertical transmission of HCMV from mother to child is a common source of infection in infants, administering VRPs to a woman who is pregnant or can become pregnant is particularly useful.

The invention provides a kit for identifying the presence of a modified HCMV gB protein within a composition, for the purification of a modified HCMV gB protein from a composition, or for detecting a modified HCMV gB protein in pre-fusion or non-post-fusion conformation; the kit comprising an antibody or antigen-binding fragment thereof that is specific for the modified HCMV gB protein. The invention further provides a pharmaceutical kit for the prevention or treatment of HCMV virus infection comprising a pharmaceutical composition comprising an effective amount of an antibody or antigen-binding fragment thereof that is specific for a modified HCMV gB protein as described herein. The kit also contains instructions for using the antibody or antigen-binding fragment thereof for identifying the presence of a modified HCMV gB protein within a composition, for the purification of a modified HCMV gB protein from a composition, or for the prevention or treatment of HCMV infection. The kit may also contain excipients, diluents, adjuvants, syringes, other appropriate means of administering the antibody or antigen-binding fragment thereof, or instructions for decontamination or disposal.

By “adjacent”, it is meant “next to” or “side-by-side”. By “immediately adjacent”, it is meant adjacent to with no material structures in between (e.g., in the context of an amino acid sequence, two residues being “immediately adjacent” to each other means there are atoms between the two residues sufficient to form the bonds necessary for a polypeptide sequence, but not a third residue (a third residue being a “material structure” in that context)).

By “c-terminally” or “c-terminal” to, it is meant toward the c-terminus. Therefore, by “c-terminally adjacent” it is meant “next to” and on the c-terminal side (i.e., on the right side if reading from left to right).

By “n-terminally” or “n-terminal” to, it is meant toward the n-terminus. Therefore, by “n-terminally adjacent” it is meant “next to” and on the n-terminal side (i.e., on the left side if reading from left to right).

By “truncated” it is meant cut short, e.g., “truncated after residue 703” herein means the amino acid sequence comprises residue 703, but the residues c-terminal thereto (i.e., 703->-C′) have been deleted. Optionally, the c-terminal (or 3′-end) of a truncated protein or polynucleotide (respectively) may be extended by operably linking it to another (heterologous) amino acid or nucleic acid sequence. In this way, the location of truncation does not always correspond with the c-terminal end (or 3′ end) of the sequence. If a gB protein or polynucleotide is “truncated” herein it is generally meant that the gB protein or polynucleotide is truncated as compared to a wild type or control gB protein or polynucleotide (i.e., it is the native amino acids or nucleic acids, found within a wild type gB protein or polynucleotide, which have been deleted).

“Operably linked” means connected, such as by genetic fusion, so as to be “operational”, for example, the configuration of polynucleotide sequences for recombinant protein expression. In certain embodiments, “operably linked” refers to the art-recognized positioning of, e.g., nucleic acid components such that the intended function (e.g., expression) is achieved. A person with ordinary skill in the art will recognize that under certain circumstances (e.g., a cleavage site or purification tag), two or more components “operably linked” together are not necessarily adjacent to each other in the nucleic acid or amino acid sequence. A coding sequence that is “operably linked” to a “control sequence” (e.g., a promoter, enhancer, or IRES) is ligated in such a way that expression of the coding sequence is under the influence or control of the control sequence, but it would be recognized in the art that such a ligation is not limited to adjacent ligation. Further, and by example, it would be understood that to construct an HCMV gB amino acid sequence into which a heterologous amino acid sequence has been inserted, the heterologous sequence must be operably linked to the native HCMV gB amino acids (likewise the polynucleotide sequence encoding the heterologous sequence must be operably linked to the polynucleotide sequence encoding the native HCMV gB amino acids). A person with ordinary skill in the art will recognize that a variety of configurations are functional and encompassed.

The terms “about” or “approximately” mean roughly, around, or in the regions of. The terms “about” or “approximately” further mean within an acceptable contextual error range for the particular value as determined by one of ordinary skill in the art, which will depend in part on how the value is measured or determined, i.e. the limitations of the measurement system or the degree of precision required for a particular purpose, e.g. the amount of a complex within media. When the terms “about” or “approximately” are used in conjunction with a numerical range, it modifies that range by extending the boundaries above and below the numerical values set forth. For example, “between about 5.5 to 6.5 mg/ml” means the boundaries of the numerical range extend below 5.5 and above 6.5 so that the particular value in question achieves the same functional result as within the range. For example, “about” and “approximately” can mean within one or more than one standard deviation as per the practice in the art. Alternatively, “about” and “approximately” can mean a range of up to 20%, preferably up to 10%, more preferably up to 5%, and more preferably up to 1% of a given value.

It is further to be understood that all base sizes or amino acid sizes, and all molecular weight or molecular mass values, given for nucleic acids or polypeptides are approximate and are provided for description. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Thus, components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not mean that the steps must be performed in that order (i.e., step 1 then step 2 then step 3, etc.). The word “then” may be used to specify the order of a method's steps.

Although methods and materials similar or equivalent to those described herein can be used in the practice or testing of this disclosure, suitable methods and materials are described below. The term “comprises” means “includes”. Thus, unless the context requires otherwise, the word “comprises”, and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or group thereof. The term “comprising” is open-ended and encompasses “consisting”, which is closed form. The abbreviation, “e.g.” is derived from the Latin exempli gratia, and is used herein to indicate a non-limiting example. Thus, the abbreviation “e.g.” is synonymous with the term “for example.”

This invention is further illustrated by the following examples which should not be construed as limiting.

### EXAMPLES

### Example 1

HCMV Merlin strain gB (SEQ ID NO: 1 and UniProtKB Accession No. F5HB53) is modified with previously described furin cleavage site modifications to reduce processing during protein expression (Chandramouli et al. 2015 Nat. Comm. 6 (8176): 1-12; Burke & Heldwein 2015 PLOS Path. DOI: 10.1371; see also WO 2012/049317 (corresponding to US 2013/0216613); and WO 2016/092460 (corresponding to US 2018/0265551). In particular, residue R457 (numbered per SEQ ID NO: 1) is substituted to serine (R457S) and residue R460 (numbered per SEQ ID NO: 1) is substituted to serine (R460S). To prevent aggregation, Fusion Loop 1 (FL1) and Fusion Loop 2 (FL2) (corresponding to residues 155-157 and 240-242, respectively, of SEQ ID NO: 1) are modified as previously described: hydrophobic residues within FL1 or FL2 are replaced with their (generally) more hydrophilic HSV-1 counterparts (Burke & Heldwein 2015 PLOS Path. DOI: 10.1371; see also WO 2012/049317 (corresponding to US 2013/0216613); and WO 2016/092460 (corresponding to US 2018/0265551)). In particular, the following substitutions are made (numbered per SEQ ID NO: 1) I156H, H157R, W240R, R457S, and R460S A HCMV Merlin strain gB having such furin cleavage site and fusion loops modifications forms a trimer and, without further modification, has post-fusion conformation (see Chandramouli et al. 2015 Nat. Comm. 6, 8176:1-12).

HCMV Merlin strain gB molecules having the above furin cleavage site modifications (R457S and R460S) and the above FL1 and FL2 modifications (1156H and H157R (FL1) with W240R (FL2)) are referred to herein as “gB′2” or ‘gB prime 2’.

HCMV AD169 strain gB (SEQ ID NO: 6) is modified to contain the furin cleavage site modifications and fusion loop modifications corresponding to those in HCMV Merlin strain gB′2. The resulting modified HCMV AD169 gB molecules are referred to herein as “gB′1” or ‘gB prime one’.

The gB′2 molecule as described above is further modified to provide gB′-6C-ΔMPR-GCN4 (SEQ ID NO: 3) by: substitution of six residues to cysteine (S269C, L656C, N524C, M684C, D217C, and S587C); truncation of the residues C-terminal to MPR residue D703; and addition of the General Control Nonderepressible (GCN) 4 Leucine Zipper domain in a trimeric oligomerization state (“GCN4” herein) having the sequence SEQ ID NO: 2 (see Protein Data Bank 4DME and Oshaben et al. 2012 Biochem. 51 (47): 9581-9591). The GCN4 sequence is placed c-terminally adjacent to D703. This modified gB molecule is referred to herein as gB′-6C-GCN4 (SEQ ID NO: 3). Such a modified gB molecule can be represented by N′-6C-AMPR-D703-GCN4-C′.

For purification purposes, gB′-6C-AMPR-GCN4 (SEQ ID NO: 3) is further modified by operably linking a TEV-cleavage site and 6×HIS-tag (SEQ ID NO: 5) to the C-terminus of the GCN4 domain, to provide SEQ ID NO: 4 (gB′-6C-AMPR-GCN4-TEV).

The gB′2 and gB′-6C-ΔMPR-GCN4 constructs are made and expressed in transiently transfected Expi293 cells. The presence of antigenic domain 5 (AD-5) epitope in gB′2 and gB′-6C-ΔMPR-GCN4 is confirmed via binding to a neutralizing antibody 1G2 (see Chandramouli et al. 2015 Nat. Comm. 6 (8176): 1-12).

Nano Differential Scanning Fluorimetry (NanoDSF) analysis was then conducted on gB′2 and gB′-6C-ΔMPR-GCN4 to determine melting temperatures. Based on previous results (data not shown), gB′2 produces one peak (hereinafter referred to as “peak I”). However, the gB′-6C-ΔMPR-GCN4 molecule produces a peak at a lower temperature than the temperature of peak I (hereinafter referred to as “peak II”).

Without wishing to be bound by theory, it is believed that the insertion/substitution of the six (6) recombinant cysteine residues (S269C, L656C, N524C, M684C, D217C, and S587C) results in three new, recombinant disulfide bonds within the modified HCMV gB molecule (S269C with L656C, N524C with M684C, and D217C with S587C) and that these new, recombinant disulfide bonds further stabilize the molecule in pre-fusion conformation such that maintaining that conformation in a purified state is possible. Combination of (1) inactivating the MPR, transmembrane domain, and cytoplasmic domains by truncation (e.g., removing the residues c-terminal to any one of D699, K700, V701, V702, and D703 numbered according to SEQ ID NO: 1), (2) operably linking a heterologous trimerization domain c-terminal to Domain V, and (3) introducing three disulfide bonds (via six amino acid substitutions to cysteine) is sufficient to disrupt the transition of the viral gB protein from pre-fusion to post-fusion conformation and, therefore, is sufficient to produce a stabilized purified HCMV gB protein in pre-fusion conformation (Towne, Merlin, or AD169 strain).

### Example 2

### Molecule Design

The following sequence design approaches were taken to design a soluble and stable prefusion glycoprotein B antigen from human cytomegalovirus (HCMV) that, unlike other approaches, does not require for example the introduction of recombinant proline residue(s) or the aid of a chemical cross-linker and fusion inhibitor. While the design methodology is described in more detail below, it is noted that all modified HCMV gB proteins of this Example comprise at least one recombinant cysteine residue.

Two macromolecular approaches were taken to design the gB molecules herein: (A) Fusion of a leucine zipper (GCN4) to a gB structure with an inactivated (via deletion) cytoplasmic domain, transmembrane domain, and membrane proximal regions (MPR) [gB #s 33, 36, 37, 40, 43, 44, 47, 48, 51, 52, 55, 56, 59, 60, 63, 64, 67, and 68]. Also (B) fusion of a trimeric aldolase structure (instead of a GCN4) to a gB structure with an inactivated (via deletion) cytoplasmic domain, transmembrane domain, and MPR [gB #s 70, 72, 75, 76, 79, and 80]. Designs comprising a GCN4 domain (i.e., (A) designs) all also comprise the optional FL1, FL2, and furin cleavage site mutations that were described above for gB′2. The designed molecules comprising an aldolase sequence (i.e., (B) designs) do not comprise the optional FL1 or FL2 mutations, but they do contain the furin cleavage site mutations. [gB #s 33, 36, 37, 40, 43, 44, 47, 48, 51, 52, 55, 56, 59, 60, 63, 64, 67, and 68 are (A) designs comprising a GCN4 sequence whereas gB #s 70, 72, 75, 76, 79, and 80 are (B) designs comprising an aldolase sequence]. Different GCN4 sequences were used, SEQ ID NO: 12 for gB #33; SEQ ID NO: 13 for gB #51; SEQ ID NO: 14 for gB #s 67, 37, and 59; SEQ ID NO: 15 for gB #s 55 and 63; SEQ ID NO: 16 for gB #43; and SEQ ID NO: 17 for gB #47. Similarly, different aldolase sequences were used, SEQ ID NO: 18 for gB #s 47, 75, and 79; SEQ ID NO: 19 for gB #s 80, 76, and 72.

The macromolecular designs (A) or (B) were then further modified at a micromolecular level by making specific amino acid residue mutations that (i) introduce disulfide bonds (i.e., through the introduction of recombinant cysteine residues) and, for some of the designs that contained recombinant cysteine residues, also (ii) introduce covalent or non-covalent bonds that strengthen the pre-fusion interface (through the substitution of a residue(s) to a, what are herein referred to as, “consensus” amino acid). As used herein, the sequence of the most frequently occurring amino acid at each position is called “the consensus sequence.” gB #s 36, 40, 44, 48, 52, 56, 60, 64, 68, 72, 76, and 80 are (i) designs containing recombinant cysteine substitutions but no consensus mutations; gB #s 33, 37, 43, 47, 51, 55, 59, 63, 67, 70, 75, and 79 are (ii) designs containing both recombinant cysteine substitutions and recombinant consensus mutations.

Ninety-three sequence designs were generated through this methodology. Select designs were then cloned, expressed, purified, and analyzed against a control gB molecule (“control gB”) in this case being a wild type Merlin gB having been truncated c-terminally to residue K700, i.e. control gB here does not comprise FL1, FL2, or furin cleavage site mutations).

### Methods

Cloning by recombinant DNA and Expression: The genes encoding the mutants were synthesized and cloned into an expression vector. For ease of purification, a space linker and HIS tag were included at the C-termini of the sequences. Designed gB constructs were expressed using Expi293 cells by following a common manufacturing protocol (Thermo Fisher Scientific, Carlsbad, CA). The expression was conducted either in a 24 deep well plate format for screening purposes, or at 100 ml small scale for production purposes. Cell culture supernatants were harvested on Day 5-6.

Expression level quantification by Bio-layer Interferometry (BLI): Octet quantification assays were performed on an Octet 96 Red system. Eight anti-HIS biosensors were presoaked in blank spent media for 10 minutes prior to measurements. 200 ul samples of control gB (postfusion gB standard) were prepared in a black 96-well plate with control gB (postfusion gB standard) diluted in media from 20 ug/ml to 0.3125 ug/ml. The binding curve of control gB (postfusion gB standard) protein and designed gB protein on anti-HIS biosensor were measured. Initial binding rates of control (postfusion gB standard) were plotted against its known concentration to generate a calibration curve. That calibration curve was used to calculate the concentration of designed gB protein in media by fitting the measured initial binding rate (of designed gB proteins) to that calibration curve. Expression levels were measured in duplicate wells of each construct's media and average readouts were recorded (data not shown).

Purification: Following the expression level results, certain designed gB constructs were selected for purification. The culture supernatants of selected gB proteins were loaded onto a 5 mL HisTrap Excel column (Cytiva Life Sciences) at a flow rate of 5 mL/min. After loading, the column was washed with buffer A (50 mM Tris pH 8.0, 300 mM NaCl) for 20 Column Volume (CV) to remove non-specially bound proteins. Elution was carried out with buffer gradient from 0% to 18% buffer B (50 mM Tris pH 8.0, 300 mM NaCl, 500 mM imidazole) within 10 column volumes. The fractions containing target proteins were pooled and concentrated using a 10K cutoff Amicon® Ultra-15 concentrator (Millipore Sigma). Samples were further purified over a pre-equilibrated superose 6 increase 10/300 column (Cytiva Life Sciences) with 20 mM HEPS, 150 mM Nacl, pH 7.5 at a flow rate of 0.5 ml/min. SDS-PAGE was used to check the molecular weight of the samples. The fractions corresponding to the targeted protein were pooled together and quantitated using the absorbance at 280 nm (based on SDS-PAGE analysis).

High-Performance Liquid Chromatography (HPLC-Size Exclusion Chromatography (SEC) analysis: HPLC-SEC was used to estimate the molecule size of purified gB proteins. 10 ul of purified gB samples were injected into a superose 6 INCREASE 3.2/300 column and evaluated using an Alliance HPLC system at a flow rate of 0.1 ml/min. UV214 readings were obtained with a Photodiode Array Detector.

Epitope integrity assessment by BIAcore Surface Plasmon Resonance (SPR) analysis: To determine whether the designed gB proteins maintain the epitopes (especially neutralizing epitopes) found within wild type/control gB proteins, analysis via BIAcore SPR was conducted. The SPR experiment was performed in a running buffer composed of 0.01 M HEPES pH 7.4, 0.15 M NaCl, 3 mM EDTA, 0.005% v/v Surfactant P20 at 25° C. using Biacore 8K (GE Healthcare). Series S protein A sensor chip (GE Healthcare) was used. Briefly, the neutralizing anti-gB monoclonal antibody 1G2 (which targets antigenic domain 5, Chandramouli et al. 2015 Nat. Comm. 6, 8176:1-12) was immobilized to a protein A sensor chip (GE Healthcare). Serial dilutions of purified gB constructs were injected ranging in concentration from 10 nM to 1.25 nM. The resulting data were fit to a 1:1 binding model using Biacore Evaluation Software (GE Healthcare). Most of the designed gB proteins showed 1G2 binding comparable to control gB except that gB #37 P2, gB #44 and gB #51 P1 showed reduced binding affinity, and gB #36 did not bind 1G2 at all. Regarding gB #36, the inventors noticed a discrepancy between the binding results measured by Octet BLI (which showed some level of binding to 1G2) and the binding results of Biacore SPR (which didn't show binding to 1G2 at all). Without wishing to be bound by theory, it is believed that the reason behind this discrepancy is that the binding of Octet was measured in raw cell media, which has a friendly environment (proper media pH and salt/chemicals conc.) to help the construct maintain its ‘good’ conformation. However, for the binding of Biacore, a fully purified construct was used. This construct protein went through two-step purifications and 1-step spin concentration and was kept at 4° C. for at least one week, which might cause the damage of conformation/epitopes on gB #36 and lead to the loss of binding to 1G2.

Nano Differential Scanning Fluorimetry (DSF) analysis: NanoDSF was used to assess the thermal stability of purified gB proteins. Samples were diluted to 0.2 mg/ml by PBS and 20 ul of each sample was loaded into capillary tubes. Temperature ramp was set to 1° C./min increase from 20° C. to 99° C. The reported values are the mean of 2nd derivative of Ratio 350/330 from two independent measurements.

### Results

The twenty-four designed (mutant) gB molecules summarized in the below Table 3 (of the eighteen GCN4 designs) and Table 4 (of the six aldolase designs) all produced peak 2 (P2) via NanoDSF analysis (presence of peak II indicates the HCMV gB protein is in pre-fusion conformation). Of those designs, some constructs produced homogenous populations (i.e., no or undetectable levels of peak 1 (P1)/post-fusion like conformation gB) while the remaining designs produced heterologous populations (i.e., produced both peaks I and II via NanoDSF). Twenty-three of the designed gB molecules that formed a pre-fusion conformation also bound the neutralizing 1G2 antibody (as discussed above, gB #36 did not bind 1G2 in these experiments). Without wishing to be bound by theory, it is believed that all of the molecules summarized in Table 3 comprise the immunological epitopes (e.g., neutralizing epitopes) of wild type HCMV gB molecules.

Table 3 provides a summary of the eighteen recombinant cysteine and consensus substitutions within the designed gB amino acid sequences comprising a GCN4 sequence SEQ ID NOs: 20-55 (see elsewhere herein for information on FL1, FL2, and furin cleavage site mutations). A column is provided for each GCN4 gB design (gB #) and a row is provided for each amino acid position at which a substitution occurs within at least one of the designs (residue numbering corresponds to the numbering of SEQ ID NO: 1). Note that at some residues, such as T100, the recombinant amino acid (i.e., that resulting from a substitution) may not be the same for all designs (e.g., a substitution at T100 may result in different recombinant amino acids being present within the designed gB molecule such as glycine (T100G) for gB #33 or cysteine (T100C) for gB #37). Two recombinant cysteine residues were introduced into gB #33 (at D217 and S587), gB #51 (at N524 and M684), gB #37 (at T100 and A267), gB #48 (at S269 and L656), gB #44 (at G251 and P614), gB #40 (at T100 and A267), gB #52 (at N524 and M684), and gB #36 (at D217 and S587); three recombinant cysteine residues were introduced into gB #43 (at T100, G251, and P614) and gB #47 (at T100, S269, and L656); four recombinant cysteine residues were introduced into gB #67 (at S269, N524, L656, and M684), gB #59 (at T100, A267, N524, and M684), gB #68 (at S269, N524, L656, and M684), gB #64 (at G251, N524, P614, and M684), gB #60 (at T100, A267, N524, and M684), and gB #56 (at D217, N524, S587, and M684); five recombinant cysteine residues were introduced into gB #55 (at T100, D217, N524, S587, and M684) and gB #63 (at T100, G251, N524, P614, and M684).

TABLE 3

| 33 | 51 | 67 | 37 | 59 | 55 | 43 | 63 | 47 |
|---|---|---|---|---|---|---|---|---|
| T100G | T100G | T100G | T100C | T100C | T100C | T100C | T100C | T100C |
| I103F | I103F | I103F | I103F | I103F | I103F | I103F | I103F | I103F |
| M114Y | | | | | | | | |
| V142I | V142I | V142I | V142I | V142I | V142I | V142I | V142I | |
| S183G | S183G | S183G | S183G | S183G | S183G | S183G | S183G | |
| A200Y | | | | | | | | |
| D217C | | | | | D217C | | | |
| L248V | | | | | | | | |
| A267V | A267V | A267V | A267C | A267C | A267V | A267V | A267V | |
| | | S267C | | | | | | S267C |
| | | | | | | G251C | G251C | |
| S314A | | | S314A | | S314A | | | |
| | Q337H | Q337H | Q337H | Q337H | Q337H | | Q337H | |
| T355V | T355V | T355V | T355V | T355V | T355V | T355V | T355V | |
| S367I | S367I | S367I | S367I | S367I | S367I | S367I | S367I | S367I |
| S377T | | | S377T | | S377T | | | |
| V382F | | | | | | | | |
| V428I | V428F | V428F | V428F | V428F | V428F | V428F | V428F | V428I |
| G433P | G433P | G433P | G433P | G433P | G433P | G433P | G433P | G433P |
| G493T | G493A | G493A | G493A | G493K | G493K | G493K | G493K | G493K |
| R497Q | | | | | | | | |
| V516I | | | | | | | | |
| F517W | F517W | F517W | F517W | F517W | F517W | F517W | F517W | F517W |
| | N524C | N524C | | N524C | N524C | | N524C | |
| K535R | | | | | | | | |
| G547A | G547A | G547A | G547A | G547A | G547A | G547A | G547A | |
| A549S | A549S | A549S | A549S | A549S | A549S | A549S | A549S | |
| S570D | S570D | S570D | S570D | S570D | S570D | S570D | S570D | |
| N583R | N583R | N583R | N583R | N583R | N583R | N583R | N583R | N583R |
| A585S | | A585E | A583S | A583S | A585E | A583S | A585E | |
| S587C | | | | | S587C | | | |
| | | | | | | P614C | P614C | |
| S624Q | S624Q | S624Q | S624Q | S624Q | S624Q | S624Q | S624Q | S624Q |
| A650D | | A650S | A650S | A650S | A650S | A650S | A650S | |
| D652N | | D652N | D652N | D652N | D652N | D652N | D652N | |
| | | L656C | | | | | | L656C |
| | M684C | M684C | | M684C | M684C | | M684C | |
| V694I | V694I | V694I | V694I | V694I | V694I | V694I | V694I | |
| | | | | | K695R | | | |

| 48 | 68 | 44 | 64 | 40 | 60 | 52 | 36 | 56 |
|---|---|---|---|---|---|---|---|---|
| | | | | T100C | T100C | | | |
| | | | | | | | D217C | D217C |
| | | | | A267C | A267C | | | |
| S267C | S267C | | | | | | | |
| | | G251C | G251C | | | | | |
| | N524C | | N524C | | N524C | N524C | | N524C |
| | | | | | | | S587C | S587C |
| | | P614C | P614C | | | | | |
| L656C | L656C | | | | | | | |
| | M684C | | M684C | | M684C | M684C | | M684C |

Table 4 provides a summary of the six recombinant cysteine and consensus substitutions within the designed gB amino acid sequences comprising an aldolase sequence SEQ ID NOs: 56-67 (see elsewhere herein for information on FL1, FL2, and furin cleavage site mutations). A column is provided for each aldolase gB design (gB #) and a row is provided for each amino acid position at which a substitution occurs within at least one of the designs (residue numbering corresponds to the numbering of SEQ ID NO: 1). Note that at some residues, such as Q98, the recombinant amino acid (i.e., that resulting from a substitution) may not be the same for all designs (e.g., a substitution at Q98 may result in different recombinant amino acids being present within the designed gB molecule such as threonine (Q98T) for gB #70 or asparagine (Q98N) for gB #79). One recombinant cysteine residue was introduced into gB #70 (at T239), gB #72 (at T239), gB #75 (at 1156) and gB #76 (at I156); two recombinant cysteine residues were introduced into gB #79 (at D217 and S587) and gB #80 (at D217 and S587).

TABLE 4

| gB#70 | gB#75 | gB#79 | gB#80 | gB#76 | gB#72 |
|---|---|---|---|---|---|
| Q98T | Q98T | Q98N | | | |
| T100G | T100G | T100G | | | |
| I103V | I103V | | | | |
| | I156C | | | I156C | |
| S181K | S181K | S181R | | | |
| S183G | S183G | S183G | | | |
| A200Y | A200Y | | | | |
| | | D217C | D217C | | |
| T239C | | | | | T239C |
| W240G | W240G | W240G | | | |
| T255V | | T255V | | | |
| | T256E | | | | |
| A267V | A267V | | | | |
| T268L | T268L | T268L | | | |
| V273I | V273I | | | | |
| N281D | N281D | N281D | | | |
| S314A | S314A | | | | |
| E350D | E350D | E350D | | | |
| S358T | | | | | |
| A360T | A360T | A360T | | | |
| S367I | S367I | S367I | | | |
| L376I | L376V | | | | |
| S377A | S377A | | | | |
| M384E | M384P | | | | |
| V428I | V428I | | | | |
| G433P | G433P | G433P | | | |
| G493K | G493K | G493E | | | |
| V516I | V516I | | | | |
| F517W | F517W | F517W | | | |
| K535R | K535R | | | | |
| F541W | F541W | | | | |
| M542L | M542L | | | | |
| L548I | L548I | L548I | | | |
| A549S | A549S | A549S | | | |
| S570D | S570D | | | | |
| N583Q | N583Q | | | | |
| | | S587C | S587C | | |
| Q591F | Q591F | Q591F | | | |
| A621T | A621T | | | | |
| A650D | A650D | A650D | | | |
| V663L | V663L | | | | |
| Y696E | Y696E | | | | |
| V697I | | | | | |
| P704N | P704N | P704N | | | |
| K710Q | K710Q | | | | |
| D714S | D714S | D714S | | | |
| | S716D | | | | |
| G717D | G717D | G717D | | | |

Table 5 provides a summary of the antigenic characterization (e.g., the binding affinity) of modified HCMV gB proteins from Example 2 bound to 1G2 antibody. Ka is “association rate constant.” Kd is “dissociation rate constant,” and KD is “equilibrium dissociation constant.” The designs without a “peak I”/“P1” or “peak 2”/“P2” label produced homogeneous populations all at peak 2 with no or undetectable levels of peak I/“P1”. The remaining designs produced heterologous populations (i.e., produced both peak I/“P1” and peak II/“P2”).

TABLE 5

| Purified gB | | | |
|---|---|---|---|
| constructs | ka (1/Ms) | kd (1/s) | KD(M) |
| gB#33 | 1.0 × 10⁵ ± 9.5 × 10² | 7.2 × 10⁻⁶ ± | 6.6 × 10⁻¹¹ ± |
| | | 2.1 × 10⁻⁷ | 2.5 × 10⁻¹² |
| gB#37 P1 | 6.0 × 10⁴ ± 1.0 × 10³ | 8.7 × 10⁻⁸ ± | 1.4 × 10⁻¹² ± |
| | | 3.5 × 10⁻⁸ | 6.0 × 10⁻¹³ |
| gB#37 P2 | 7.5 × 10⁴ ± 2.5 × 10⁴ | 6.6 × 10⁻⁵ ± | 8.3 × 10⁻¹⁰ ± |
| | | 4.2 × 10⁻⁵ | 2.8 × 10⁻¹⁰ |
| gB#40 P1 | 1.0 × 10⁵ ± 2.6 × 10³ | 3.8 × 10⁻⁷ ± | 3.6 × 10⁻¹² ± |
| | | 2.2 × 10⁻⁸ | 3.0 × 10⁻¹³ |
| gB#40 P2 | 8.2 × 10⁴ ± 0.0 × 10⁰ | 2.9 × 10⁻⁷ ± | 3.5 × 10⁻¹² ± |
| | | 0.0 × 10⁺⁰ | 0.0 × 10⁺⁰ |
| gB#43 P1 | 3.7 × 10⁴ ± 1.1 × 10⁴ | 5.4 × 10⁻⁸ ± | 1.5 × 10⁻¹² ± |
| | | 2.0 × 10⁻⁹ | 4.3 × 10⁻¹³ |
| gB#43 P2 | 3.2 × 10⁴ ± 1.2 × 10³ | 2.3 × 10⁻⁷ ± | 7.2 × 10⁻¹² ± |
| | | 3.9 × 10⁻⁸ | 1.5 × 10⁻¹² |
| gB#44 | 1.0 × 10⁴ ± 4.5 × 10³ | 6.8 × 10⁻⁶ ± | 6.9 × 10⁻¹⁰ ± |
| | | 1.6 × 10⁻⁶ | 1.4 × 10⁻¹⁰ |
| gB#47 P1 | 7.3 × 10⁴ ± 7.7 × 10³ | 2.9 × 10⁻⁷ ± | 4.1 × 10⁻¹² ± |
| | | 1.9 × 10⁻⁷ | 3.1 × 10⁻¹² |
| gB#47 P2 | 7.4 × 10⁴ ± 8.3 × 10³ | 3.1 × 10⁻⁶ ± | 3.9 × 10⁻¹¹ ± |
| | | 3.7 × 10⁻⁶ | 4.6 × 10⁻¹¹ |
| gB#48 P1 | 9.7 × 10⁴ ± 1.9 × 10⁴ | 1.9 × 10⁻⁶ ± | 2.3 × 10⁻¹¹ ± |
| | | 2.4 × 10⁻⁶ | 2.9 × 10⁻¹¹ |
| gB#48 P2 | 1.1 × 10⁵ ± 0.0 × 10⁰ | 1.4 × 10⁻⁶ ± | 1.2 × 10⁻¹¹ ± |
| | | 0.0 × 10⁺⁰ | 0.0 × 10⁺⁰ |
| gB#51 P1 | 1.3 × 10⁵ ± 1.1 × 10³ | 7.0 × 10⁻⁵ ± | 5.3 × 10⁻¹⁰ ± |
| | | 1.8 × 10⁻⁵ | 1.4 × 10⁻¹⁰ |
| gB#51 P2 | 1.2 × 10⁵ ± 3.1 × 10³ | 5.3 × 10⁻⁶ ± | 4.3 × 10⁻¹¹ ± |
| | | 4.7 × 10⁻⁶ | 3.9 × 10⁻¹¹ |
| gB#52 P1 | 1.4 × 10⁵ ± 0.0 × 10⁰ | 5.3 × 10⁻⁷ ± | 3.7 × 10⁻¹² ± |
| | | 0.0 × 10⁺⁰ | 0.0 × 10⁺⁰ |
| gB#52 P2 | 1.1 × 10⁵ ± 3.4 × 10³ | 1.3 × 10⁻⁵ ± | 1.2 × 10⁻¹⁰ ± |
| | | 5.9 × 10⁻⁶ | 5.8 × 10⁻¹¹ |
| gB#55 P1 | 8.3 × 10⁴ ± 2.9 × 10³ | 1.5 × 10⁻⁷ ± | 1.9 × 10⁻¹² ± |
| | | 5.7 × 10⁻⁸ | 7.5 × 10⁻¹³ |
| gB#55 P2 | 1.0 × 10⁵ ± 3.9 × 10² | 4.1 × 10⁻⁶ ± | 4.1 × 10⁻¹¹ ± |
| | | 4.7 × 10⁻⁶ | 4.7 × 10⁻¹¹ |
| gB#56 P1 | 9.2 × 10⁴ ± 2.8 × 10³ | 2.2 × 10⁻⁷ ± | 2.3 × 10⁻¹² ± |
| | | 1.2 × 10⁻⁷ | 1.3 × 10⁻¹² |
| gB#56 P2 | 9.1 × 10⁴ ± 0.0 × 10⁰ | 1.0 × 10⁻⁷ ± | 1.1 × 10⁻¹² ± |
| | | 0.0 × 10⁺⁰ | 0.0 × 10⁺⁰ |
| gB#59 P1 | 6.3 × 10⁴ ± 0.0 × 10⁰ | 1.6 × 10⁻⁷ ± | 2.6 × 10⁻¹² ± |
| | | 0.0 × 10⁺⁰ | 0.0 × 10⁺⁰ |
| gB#59 P2 | 4.0 × 10⁴ ± 2.7 × 10³ | 1.6 × 10⁻⁷ ± | 4.0 × 10⁻¹² ± |
| | | 6.4 × 10⁻⁸ | 1.3 × 10⁻¹² |
| gB#60 P1 | 1.1 × 10⁵ ± 1.7 × 10⁴ | 5.2 × 10⁻⁶ ± | 4.4 × 10⁻¹¹ ± |
| | | 3.3 × 10⁻⁷ | 3.6 × 10⁻¹² |
| gB#60 P2 | 1.0 × 10⁵ ± 5.2 × 10³ | 1.1 × 10⁻⁵ ± | 1.1 × 10⁻¹⁰ ± |
| | | 8.4 × 10⁻⁷ | 2.5 × 10⁻¹² |
| gB#63 P1 | 8.5 × 10⁴ ± 2.1 × 10⁴ | 1.4 × 10⁻⁶ ± | 1.9 × 10⁻¹¹ ± |
| | | 1.4 × 10⁻⁶ | 2.2 × 10⁻¹¹ |
| gB#63 P2 | 7.5 × 10⁴ ± 1.2 × 10⁴ | 9.8 × 10⁻⁶ ± | 1.3 × 10⁻¹⁰ ± |
| | | 2.7 × 10⁻⁷ | 1.8 × 10⁻¹¹ |
| gB#64 P2 | 5.2 × 10⁴ ± 3.4 × 10³ | 8.7 × 10⁻⁶ ± | 1.6 × 10⁻¹⁰ ± |
| | | 4.1 × 10⁻⁷ | 3.1 × 10⁻¹² |
| gB#67 P1 | 1.0 × 10⁵ ± 5.2 × 10³ | 5.9 × 10⁻⁶ ± | 5.8 × 10⁻¹¹ ± |
| | | 7.1 × 10⁻⁷ | 4.0 × 10⁻¹² |
| gB#67 p2 | 1.1 × 10⁵ ± 2.1 × 10⁴ | 6.3 × 10⁻⁶ ± | 5.6 × 10⁻¹¹ ± |
| | | 1.3 × 10⁻⁷ | 9.6 × 10⁻¹² |
| gB#68 P1 | 1.0 × 10⁵ ± 0.0 × 10⁰ | 2.3 × 10⁻⁷ ± | 2.3 × 10⁻¹² ± |
| | | 0.0 × 10⁺⁰ | 0.0 × 10⁺⁰ |
| gB#68 P2 | 1.2 × 10⁵ ± 6.2 × 10² | 1.4 × 10⁻⁵ ± | 1.2 × 10⁻¹⁰ ± |
| | | 3.6 × 10⁻⁷ | 2.3 × 10⁻¹² |
| gB#70 P1 | 1.1 × 10⁵ ± 3.2 × 10³ | 4.1 × 10⁻⁶ ± | 3.7 × 10⁻¹¹ ± |
| | | 1.8 × 10⁻⁷ | 5.7 × 10⁻¹³ |
| gB#70 P2 | 1.3 × 10⁵ ± 1.0 × 10³ | 1.4 × 10⁻⁵ ± | 1.0 × 10⁻¹⁰ ± |
| | | 3.7 × 10⁻⁷ | 3.5 × 10⁻¹² |
| gB#72 P1 | 9.4 × 10⁴ ± 1.8 × 10³ | 2.6 × 10⁻⁸ ± | 2.7 × 10⁻¹³ ± |
| | | 8.8 × 10⁻⁹ | 9.8 × 10⁻¹⁴ |
| gB#72 P2 | 1.3 × 10⁵ ± 5.1 × 10³ | 5.5 × 10⁻⁸ ± | 4.1 × 10⁻¹³ ± |
| | | 4.0 × 10⁻⁸ | 3.1 × 10⁻¹³ |
| gB#75 P1 | 1.3 × 10⁵ ± 2.0 × 10³ | 4.8 × 10⁻⁶ ± | 3.5 × 10⁻¹¹ ± |
| | | 8.5 × 10⁻⁸ | 1.1 × 10⁻¹² |
| gB#75 P2 | 1.6 × 10⁵ ± 2.4 × 10³ | 1.3 × 10⁻⁵ ± | 8.2 × 10⁻¹¹ ± |
| | | 4.4 × 10⁻⁷ | 1.4 × 10⁻¹² |
| gB#76 P1 | 9.6 × 10⁴ ± 2.8 × 10³ | 4.3 × 10⁻⁶ ± | 4.5 × 10⁻¹¹ ± |
| | | 1.8 × 10⁻⁷ | 5.2 × 10⁻¹³ |
| gB#76 P2 | 1.3 × 10⁵ ± 6.2 × 10⁰ | 1.4 × 10⁻⁵ ± | 1.0 × 10⁻¹⁰ ± |
| | | 8.1 × 10⁻⁷ | 5.9 × 10⁻¹² |
| gB#79 P1 | 8.5 × 10⁴ ± 5.9 × 10³ | 2.8 × 10⁻⁶ ± | 3.3 × 10⁻¹¹ ± |
| | | 8.3 × 10⁻⁷ | 7.4 × 10⁻¹² |
| gB#79 P2 | 1.1 × 10⁵ ± 1.0 × 10³ | 1.3 × 10⁻⁵ ± | 1.1 × 10⁻¹⁰ ± |
| | | 4.2 × 10⁻⁷ | 2.6 × 10⁻¹² |
| gB#80 P1 | 1.0 × 10⁴ ± 1.4 × 10² | 1.4 × 10⁻⁷ ± | 1.4 × 10⁻¹¹ ± |
| | | 6.5 × 10⁻⁹ | 8.3 × 10⁻¹³ |
| gB#80 P2 | 1.1 × 10⁵ ± 2.3 × 10³ | 1.3 × 10⁻⁵ ± | 1.1 × 10⁻¹⁰ ± |
| | | 4.5 × 10⁻⁷ | 1.6 × 10⁻¹² |
| gB#88 | 6.0 × 10⁴ ± 2.0 × 10³ | 8.0 × 10⁻⁶ ± | 1.3 × 10⁻¹⁰ ± |
| | | 1.5 × 10⁻⁷ | 2.0 × 10⁻¹² |
| gB#89 P1 | 1.0 × 10⁵ ± 0.0 × 10⁰ | 2.4 × 10⁻⁷ ± | 2.3 × 10⁻¹² ± |
| | | 0.0 × 10⁺⁰ | 0.0 × 10⁺⁰ |
| gB#89 P2 | 1.5 × 10⁵ ± 1.3 × 10³ | 6.3 × 10⁻⁶ ± | 4.0 × 10⁻¹¹ ± |
| | | 3.0 × 10⁻⁶ | 1.9 × 10⁻¹¹ |
| gB#90 | 2.0 × 10⁵ ± 1.3 × 10³ | 8.1 × 10⁻⁶ ± | 3.9 × 10⁻¹¹ ± |
| | | 6.7 × 10⁻⁷ | 3.5 × 10⁻¹² |

*gB#36 didn't show binding to 1G2 mAb in this experiment.

Table 6 shows the results of the thermal stability (Tm₁ value (C)) of purified gB constructs using NanoDSF. P1 refers to peak I and P2 refers to peak II. Table 6 demonstrates that most of the modified purified gB constructs of Example 2 have a lower Tm₁ value in comparison with the post-fusion gB protein. Lower Tm₁ indicates a prefusion-like conformation.

TABLE 6

| gB | Tm₁ value |
|---|---|
| constructs | (° C.) |
| gB #33 | 70.4 |
| gB #36 | 71.3 |
| gB #37 P1 | 65.3 |
| gB #37 P2 | 66 |
| gB #40 P1 | 70.6 |
| gB #40 P2 | 65.7 |
| gB #43 P1 | 68 |
| gB #43 P2 | 68 |
| gB #44 | 66 |
| gB #47 P1 | 63.6 |
| gB #47 P2 | 61.8 |
| gB #48 P1 | 66 |
| gB #48 P2 | 63 |
| gB #51 P1 | 61.4 |
| gB #51 P2 | 60.7 |
| gB #52 P1 | 58.3 |
| gB #52 P2 | 56.5 |
| gB #55 P1 | 65 |
| gB #55 P2 | 65 |
| gB #56 P1 | 64.5 |
| gB #56 P2 | 64.7 |
| gB #59 P1 | 61.5 |
| gB #59 P2 | 60.6 |
| gB #60 P1 | 60.5 |
| gB #60 P2 | 60 |
| gB #63 P1 | 67.4 |
| gB #63 P2 | 68 |
| gB #64 P2 | 63.4 |
| gB #67 P1 | 67 |
| gB #67 P2 | 65.9 |
| gB #68 P2 | 65.4 |
| gB #70 P1 | 38 |
| gB #72 P2 | 70 |
| gB #75 P1 | 37.3 |
| gB #75 P2 | 37.8 |
| gB #76 P1 | 66 |
| gB #76 P2 | 80 |
| gB #79 P1 | 66 |
| gB #79 P2 | 66.5 |
| gB #80 P1 | 71.5 |
| gB #80 P2 | 76 |
| gB #88 | 76.6 |
| gB #89 P1 | 71 |
| gB #89 P2 | N/A |
| gB #90 | 61.6 |
| Post | 75 |
| Fusion gB | |
| protein | |

The various features which are referred to in individual sections above apply, as appropriate, to other sections. Consequently, features specified in one section may be combined with features specified in other sections, as appropriate. Those skilled in the art will recognize or be able to ascertain using no more than routine experimentation, many equivalents to the specific embodiments of the invention (or aspects of the disclosure) described herein. Such equivalents are intended to be encompassed by the present application.

### EXPLANATION OF SEQUENCES

```
Amino acid sequence of wild type, Merlin strain, Human
Cytomegalovirus gB protein. See Chandramouli et al. 2015
Nat. Comm. 6(8176) at FIG. 1: See also UniProtKB Accession
No. F5HB53 (GB_HCMVM; sequence version 1 last updated
Jun. 28, 2011).
Predicted Signal peptide residues M1-A22 (double
underlined);
Fusion Loop 1 residues ¹⁵⁵YIH¹⁵⁷ (underlined);
Fusion Loop 2 residues ²⁴⁰WLY²⁴² (underlined);
Furin Cleavage Site residues R457 and R460 (underlined);
Domain III Coiled-coil Region residues N478-1523
underlined with residues N478-R511 being double underlined;
D217, S269, N524, S587, L656, and M684 (bold);
MPR residues D699-P751 residues (underlined) with residue
K724 in bold;
Transmembrane domain residues F752-Y772 (double underlined);
Cytoplasmic domain is from residues T773-V907.
SEQ ID NO: 1
                10 20 30 40 50 60
MESRIWCLVV CVNLCIVCLG AAVSSSSTRG TSATHSHHSS HTTSAAHSRS GSVSQRVTSS
                70 80 90 100 110 120
QTVSHGVNET IYNTTLKYGD VVGVNTTKYP YRVCSMAQGT DLIRFERNIV CTSMKPINED
              130 140 150 160 170 180
LDEGIMVVYK RNIVAHTFKV RVYQKVLTFR RSYAYIHTTY LLGSNTEYVA PPMWEIHHIN
              190 200 210 220 230 240
SHSQCYSSYS RVIAGTVFVA YHRDSYENKT MQLMPDDYSN THSTRYVTVK DQWHSRGSTW
              250 260 270 280 290 300
LYRETCNLNC MVTITTARSK YPYHFFATST GDVVDISPFY NGTNRNASYF GENADKFFIF
              310 320 330 340 350 360
PNYTIVSDFG RPNSALETHR LVAFLERADS VISWDIQDEK NVTCQLIFWE ASERTIRSEA
              370 380 390 400 410 420
EDSYHFSSAK MTATFLSKKQ EVNMSDSALD CVRDEAINKL QQIFNTSYNQ TYEKYGNVSV
              430 440 450 460 470 480
FETTGGLVVF WQGIKQKSLV ELERLANRSS LNLTHNRTKR STDGNNATHL SNMESVHNLV
              490 500 510 520 530 540
YAQLQFTYDT LRGYINRALA QIAEAWCVDQ RRTLEVEKEL SKINPSAILS AIYNKPIAAR
              550 560 570 580 590 600
FMGDVLGLAS CVTINQTSVK VLRDMNVKES PGRCYSRPVV IFNFANSSYV QYGQLGEDNE
              610 620 630 640 650 660
ILLGNHRTEE CQLPSLKIFI AGNSAYEYVD YLFKRMIDLS SISTVDSMIA LDIDPLENTD
              670 680 690 700 710 720
FRVLELYSQK ELRSSNVEDL EEIMREFNSY KQRVKYVEDK VVDPLPPYLK GLDDLMSGLG
              730 740 750 760 770 780
AAGKAVGVAI GAVGGAVASV VEGVATFLKN PFGAFTIILV AIAVVIITYL IYTRQRRLCT
              790 800 810 820 830 840
QPLQNLFPYL VSADGTTVTS GSTKDTSLQA PPSYEESVYN SGRKGPGPPS SDASTAAPPY
              850 860 870 880 890 900
TNEQAYQMLL ALARLDAEQR AQQNGTDSLD GRIGTQDKGQ KPNLLDRLRH RKNGYRHLKD
SDEEENV
Amino acid sequence of GCN4 Leucine Zipper Domain in a
trimeric oligomerization state (see Oshaben et al., 2012
Biochemistry 51(47): 9581-9591; see also Protein Data Bank
(PDB) Accession 4DME)
SEQ ID NO: 2
                10 20 30
RMKQLEDKVE ELLSKNYHLE NEVARLKKLV GER
(gB′-6C-ΔMPR-GCN4) - exemplary amino acid sequence of a
modified HCMV gB protein herein, as compared to wild type
Merlin HCMV gB (SEQ ID NO: 1):
Predicted Signal peptide residues M1-A22 (underlined, would
be removed in mature sequence);
Fusion Loop 1 modifications ¹⁵⁶IH¹⁵⁷ to ¹⁵⁶HR¹⁵⁷ (underlined);
Fusion Loop 2 modifications W240R (underlined);
Furin Cleavage Site residues R457S and R460S (underlined);
Substitutions D217C, S269C, N524C, S587C, L656C, and M684C
(bold and underlined);
A truncation of the residues c-terminal to MPR residue D703
(residue in bold) (i.e., cleavage of the c-terminal end of
the MPR and removal of the transmembrane and cytoplasmic
domains);
Insertion of GCN4 sequence SEQ ID NO: 2 c-terminally
adjacent to D703;
This sequence may be further modified by operably linking
a TEV cleavage site and 6xHis tag (SEQ ID NO: 5) to the
C-terminus of the GCN4 sequence, as is shown in SEQ ID
NO: 4.
SEQ ID NO: 3
                10 20 30 40 50 60
MESRIWCLVV CVNLCIVCLG AAVSSSSTRG TSATHSHHSS HTTSAAHSRS GSVSQRVTSS
                70 80 90 100 110 120
QTVSHGVNET IYNTTLKYGD VVGVNTTKYP YRVCSMAQGT DLIRFERNIV CTSMKPINED
              130 140 150 160 170 180
LDEGIMVVYK RNIVAHTFKV RVYQKVLTFR RSYAYHRTTY LLGSNTEYVA PPMWEIHHIN
              190 200 210 220 230 240
SHSQCYSSYS RVIAGTVFVA YHRDSYENKT MQLMPDCYSN THSTRYVTVK DQWHSRGSTR
              250 260 270 280 290 300
LYRETCNLNC MVTITTARSK YPYHFFATCT GDVVDISPFY NGINRNASYF GENADKFFIF
              310 320 330 340 350 360
PNYTIVSDFG RPNSALETHR LVAFLERADS VISWDIQDEK NVTCQLTFWE ASERTIRSEA
              370 380 390 400 410 420
EDSYHFSSAK MTATFLSKKQ EVNMSDSALD CVRDEAINKL QQIFNTSYNQ TYEKYGNVSV
              430 440 450 460 470 480
FETTGGLVVF WQGIKQKSLV ELERLANRSS LNLTHNSTKS STDGNNATHL SNMESVHNLV
              490 500 510 520 530 540
YAQLQFTYDT LRGYINRALA QIAEAWCVDQ RRTLEVFKEL SKICPSAILS AIYNKPIAAR
              550 560 570 580 590 600
FMGDVLGLAS CVTINQTSVK VLRDMNVKES PGRCYSRPVV IFNFANCSYV QYGQLGEDNE
              610 620 630 640 650 660
ILLGNHRTEE CQLPSLKIFI AGNSAYEYVD YLFKRMIDLS SISTVDSMIA LDIDPCENTD
              670 680 690 700 710 720
FRVLELYSQK ELRSSNVEDL EEICREFNSY KQRVKYVEDK VVDRMKQLED KVEELLSKNY
              730 736
HLENEVARLK KLVGER
(gB′-6C-ΔMPR-GCN4) - exemplary amino acid sequence of a
modified HCMV gB protein herein, as compared to wild type
Merlin HCMV gB (SEQ ID NO: 1):
Predicted Signal peptide residues M1-A22 (underlined,
would be removed in mature sequence);
Fusion Loop 1 modifications ¹⁵⁶IH¹⁵⁷ to ¹⁵⁶HR¹⁵⁷ (underlined);
Fusion Loop 2 modifications W240R (underlined);
Furin Cleavage Site residues R457S and R460S (underlined);
Substitutions D217C, S269C, N524C, S587C, L656C, and M684C
(bold and underlined);
A truncation of the residues c-terminal to MPR residue D703
(residue in bold) (i.e., cleavage of the c-terminal end of
the MPR and removal of the transmembrane and cytoplasmic
domains);
Insertion of GCN4 sequence SEQ ID NO: 2 c-terminally
adjacent to D703;
Insertion of SEQ ID NO: 5 (i.e., a TEV cleavage site
(underlined) and 6xHis tag (double underlined)
C-terminally adjacent to the GCN4 sequence).
SEQ ID NO: 4
                10 20 30 40 50 60
MESRIWCLVV CVNLCIVCLG AAVSSSSTRG TSATHSHHSS HTTSAAHSRS GSVSQRVTSS
                70 80 90 100 110 120
QTVSHGVNET IYNTTLKYGD VVGVNTTKYP YRVCSMAQGT DLIRFERNIV CTSMKPINED
              130 140 150 160 170 180
LDEGIMVVYK RNIVAHTFKV RVYQKVLTFR RSYAYHRTTY LLGSNTEYVA PPMWEIHHIN
              190 200 210 220 230 240
SHSQCYSSYS RVIAGTVFVA YHRDSYENKT MQLMPDCYSN THSTRYVTVK DQWHSRGSTR
              250 260 270 280 290 300
LYRETCNLNC MVTITTARSK YPYHFFATCT GDVVDISPFY NGTNRNASYF GENADKFFIF
              310 320 330 340 350 360
PNYTIVSDFG RPNSALETHR LVAFLERADS VISWDIQDEK NVTCQLTEWE ASERTIRSEA
              370 380 390 400 410 420
EDSYHFSSAK MTATFLSKKQ EVNMSDSALD CVRDEAINKL QQIFNTSYNQ TYEKYGNVSV
              430 440 450 460 470 480
FETTGGLVVF WQGIKQKSLV ELERLANRSS LNLTHNSTKS STDGNNATHL SNMESVHNLV
              490 500 510 520 530 540
YAQLQFTYDT LRGYINRALA QIAEAWCVDQ RRTLEVFKEL SKICPSAILS AIYNKPIAAR
              550 560 570 580 590 600
FMGDVLGLAS CVTINQTSVK VLRDMNVKES PGRCYSRPVV IFNFANCSYV QYGQLGEDNE
              610 620 630 640 650 660
ILLGNHRTEE CQLPSLKIFI AGNSAYEYVD YLFKRMIDLS SISTVDSMIA LDIDPCENTD
              670 680 690 700 710 720
FRVLELYSQK ELRSSNVEDL EEICREFNSY KQRVKYVEDK VVDRMKQLED KVEELLSKNY
              730 740 750
HLENEVARLK KLVGERENLY FQGGHHHHHH
a TEV cleavage site (underlined) and 6xHis tag
(double underlined).
SEQ ID NO: 5
                10
ENLYFQGGHH HHHH
AD169 Strain HCMV gB amino acid sequence corresponding
to UniProtKB Accession P06473 (version 1 of sequence
last updated Jan. 1, 1988; see also FIG. 2 of Burke &
Heldwein 2015 PLOS Path. DOI: 10.1371).
Domain III Coiled-coil Region residues N477-1522,
corresponding to residues N478-1523 of SEQ ID NO: 1
(FIG. 2A), underlined and residues N477-R510,
corresponding to residues N478-R511 of SEQ ID NO: 1,
double underlined;
D217, S269, N523, S586, L655, and M683 (bold);
MPR residues D698-P750, corresponding to residues
D699-P751 of SEQ ID NO: 1 (FIG. 2B) double underlined
with residue D702, which corresponds to residue K724
of SEQ ID NO: 1, in bold.
SEQ ID NO: 6
                10 20 30 40 50
MESRIWCLVV CVNLCIVCLG AAVSSSSTSH ATSSTHNGSH TSRTTSAQTR
                60 70 80 90 100
SVYSQHVTSS EAVSHRANET IYNTTLKYGD VVGVNTTKYP YRVCSMAQGT
              110 120 130 140 150
DLIRFERNII CTSMKPINED LDEGIMVVYK RNIVAHTFKV RVYQKVLTFR
              160 170 180 190 200
RSYAYIYTTY LLGSNTEYVA PPMWEIHHIN KFAQCYSSYS RVIGGTVFVA
              210 220 230 240 250
YHRDSYENKT MQLIPDDYSN THSTRYVTVK DQWHSRGSTW LYRETCNLNC
              260 270 280 290 300
MLTITTARSK YPYHFFATST GDVVYISPFY NGTNRNASYF GENADKFFIF
              310 320 330 340 350
PNYTIVSDFG RPNAAPETHR LVAFLERADS VISWDIQDEK NVTCQLIFWE
              360 370 380 390 400
ASERTIRSEA EDSYHFSSAK MTATFLSKKQ EVNMSDSALD CVRDEAINKL
              410 420 430 440 450
QQIFNTSYNQ TYEKYGNVSV FETSGGLVVF WQGIKQKSLV ELERLANRSS
              460 470 480 490 500
LNITHRTRRS TSDNNTTHLS SMESVHNLVY AQLQFTYDTL RGYINRALAQ
              510 520 530 540 550
IAEAWCVDQR RTLEVFKELS KINPSAILSA IYNKPIAARF MGDVLGLASC
              560 570 580 590 600
VTINQTSVKV LRDMNVKESP GRCYSRPVVI FNFANSSYVQ YGQLGEDNEI
              610 620 630 640 650
LLGNHRTEEC QLPSLKIFIA GNSAYEYVDY LFKRMIDLSS ISTVDSMIAL
              660 670 680 690 700
DIDPLENTDF RVLELYSQKE LRSSNVEDLE EIMREFNSYK QRVKYVEDKV
              710 720 730 740 750
VDPLPPYLKG LDDLMSGLGA AGKAVGVAIG AVGGAVASVV EGVATELKNP
              760 770 780 790 800
FGAFTIILVA IAVVIITYLI YTRQRRLCTQ PLQNLFPYLV SADGTTVTSG
              810 820 830 840 850
STKDTSLQAP PSYEESVYNS GRKGPGPPSS DASTAAPPYT NEQAYQMLLA
              860 870 880 890 900
LARLDAEQRA QQNGTDSLDG QTGTQDKGQK PNLLDRLRHR KNGYRHLKDS
DEEENV
FLAG tag
SEQ ID NO: 7
DYKDDDDK
Streptavidin tag
SEQ ID NO: 8
AWRHPQFGG
Streptavidin tag
SEQ ID NO: 9
WSHPQFEK
Streptavidin tag
SEQ ID NO: 10
WSHPQFEKGGGSGGGSGGGSWSHPQFEK
(AD169-6C-ΔMPR-GCN4) - exemplary amino acid
sequence of a modified HCMV AD169 strain gB
protein herein, as compared to HCMV AD169
gB sequence SEQ ID NO: 6 above:
Predicted Signal peptide residues M1-A22
(underlined, would be removed in mature
sequence);
Fusion Loop 1 modifications ¹⁵⁶TH¹⁵⁷ to ¹⁵⁶HR¹⁵⁷
(underlined);
Fusion Loop 2 modifications W240R (underlined);
Furin Cleavage Site modifications of residues
⁴⁵⁶RTRR⁴⁵⁹ to ⁴⁵⁶TTQT⁴⁵⁹ (underlined);
A truncation of the residues c-terminal to MPR
residue D702 (residue in bold) (i.e., cleavage
of the C-terminal end of the MPR and removal of
the transmembrane and cytoplasmic domains);
Substitutions D217C, S269C, N523C, S586C, L655C,
and M683C (bold and underline);
Insertion of GCN4 sequence SEQ ID NO: 2
C-terminally adjacent to the c-terminal-
most inserted proline residue (double
underlined);
Insertion of SEQ ID NO: 5 (i.e., a TEV cleavage
site (underlined) and 6xHis tag (double
underlined) C-terminally adjacent to the GCN4
sequence).
SEQ ID NO: 11
                10 20 30 40 50 60
MESRIWCLVV CVNLCIVCLG AAVSSSSTSH ATSSTHNGSH TSRTTSAQTR SVYSQHVTSS
                70 80 90 100 110 120
EAVSHRANET IYNTTLKYGD VVGVNTTKYP YRVCSMAQGT DLIRFERNII CTSMKPINED
              130 140 150 160 170 180
LDEGIMVVYK RNIVAHTFKV RVYQKVLTFR RSYAYHRTTY LLGSNTEYVA PPMWEIHHIN
              190 200 210 220 230 240
KFAQCYSSYS RVIGGTVFVA YHRDSYENKT MQLIPDCYSN THSTRYVTVK DQWHSRGSTR
              250 260 270 280 290 300
LYRETCNLNC MLTITTARSK YPYHFFATCT GDVVYISPFY NGINRNASYF GENADKFFIF
              310 320 330 340 350 360
PNYTIVSDFG RPNAAPETHR LVAFLERADS VISWDIQDEK NVTCQLTFWE ASERTIRSEA
              370 380 390 400 410 420
EDSYHFSSAK MTATFLSKKQ EVNMSDSALD CVRDEAINKL QQIFNTSYNQ TYEKYGNVSV
              430 440 450 460 470 480
FETSGGLVVF WQGIKQKSLV ELERLANRSS LNITHTTQTS TSDNNTTHLS SMESVHNLVY
              490 500 510 520 530 540
AQLQFTYDTL RGYINRALAQ IAEAWCVDQR RTLEVFKELS KICPSAILSA IYNKPIAARF
              550 560 570 580 590 600
MGDVLGLASC VTINQTSVKV LRDMNVKESP GRCYSRPVVI FNFANCSYVQ YGQLGEDNEI
              610 620 630 640 650 660
LLGNHRTEEC QLPSLKIFIA GNSAYEYVDY LFKRMIDLSS ISTVDSMIAL DIDPCENTDF
              670 680 690 700 710 720
RVLELYSQKE LRSSNVEDLE EICREFNSYK QRVKYVEDKV VDRMKQLEDK VEELLSKNYH
              730 740 749
LENEVARLKK LVGERENLYF QGGHHHHHH
A GCN4 amino acid sequence (see gB#33)
SEQ ID NO: 12
                10 20 30 33
RIKELEDRVE ELLSELKHLE QEVARLKKLV GER
A GCN4 amino acid sequence (see gB#051)
SEQ ID NO: 13
                10 20 30 33
RIKQLEDKVE ELLSKLKHLE QEVARLKKLV GER
A GCN4 amino acid sequence (see gB#s 67, 37, 59)
SEQ ID NO: 14
                10 20 30 33
RIKQLEDRVE ELLSKLKHLE QEVARLKKLV GER
A GCN4 amino acid sequence (see gB#s 55, 63)
SEQ ID NO: 15
                10 20 30 33
RIKQLEDRVD ELLSKLKHLE QEVARLKKLV GER
A GCN4 amino acid sequence (see gB#43)
SEQ ID NO: 16
                10 20 30 33
RIKQLEDRVE ELLSKLKHLE QEVARLKKLV GER
A GCN4 amino acid sequence (see gB#47)
SEQ ID NO: 17
                10 20 30 33
RLKQLEDKVE ELLSKLYHLE QEVARLKKLV GER
An aldolase amino acid sequence (see gB#s 70, 75,
and 79)
SEQ ID NO: 18
                10 20 30 40 50 60
MEELFKKHKI VAVLRANSVE EAKEKALALF EGGVHLIEIT FTVPDADKVI KELSELKEKG
                70 80 90 100 110 120
AIIGAGTVTS VEQCRKAICS GAEFIVSPHL DEEISRFCKE KGVFYMPGVM TPTELVKALK
              130 140 150 160 170 180
LGHTILKLFP GEVVGPQFVK AMKGPFPNVK FVPTGGVNLD NVCEWFKAGV LAVGVGSALV
              190 200 201
KGTPDEVREK AKAFVEKIRG C
An aldolase amino acid sequence (see gB#s 80, 76,
and 72)
SEQ ID NO: 19
                10 20 30 40 50 60
MEELFKKHKI VAVLRANSVE EAKEKALAVF EGGVHLIEIT FTVPDADTVI KELSELKEKG
                70 80 90 100 110 120
AIIGAGTVTS VEQCRKACES GAEFIVSPHL DEEISQFCKE KGVFYMPGVM TPTELVKAMK
              130 140 150 160 170 180
LGHTILKLFP GEVVGPQFVK AMKGPFPNVK FVPTGGVNLD NVCEWFKAGV LAVGVGSALV
              190 200 201
KGTPDEVREK AKAFVEKIRG C
amino acid sequence for design gB#33 (predicted
signal sequence underlined)
SEQ ID NO: 20
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSYKPINEDLDEGIMVVYKRNIVAHTFKVRIYQKVLTFR
RSYAYHRTTYLLGSNTEYVAPPMWEIHHINSHGQCYSSYSRVIAGTVFVYYHRDSYENKTMQLMPDCYSNTHSTR
YVTVKDQWHSRGSTRLYRETCNVNCMVTITTARSKYPYHFFVTSTGDVVDISPFYNGTNRNASYFGENADKFFIF
PNYTIVSDFGRPNAALETHRLVAFLERADSVISWDIQDEKNVTCQLTFWEASERVIRSENEDSYHFISAKMTATF
LTKKQEFNMSDSALDCVRDEAINKLQQIFNTSYNQTYEKYGNVSVFETTGGLIVFWQPIKQKSLVELERLANRSS
LNLTHNSTKSSTDGNNATHLSNMESVHNLVYAQLQFTYDTLRTYINQALAQIAEAWCVDQRRTLEIWKELSKINP
SAILSAIYNRPIAARFMGDVLALSSCVTINQTSVKVLRDMNVKEDPGRCYSRPVVIFRFSNCSYVQYGQLGEDNE
ILLGNHRTEECQLPSLKIFIAGNQAYEYVDYLFKRMIDLSSISTVDSMIDLNIDPLENTDERVLELYSQKELRSS
NVFDLEEIMREFNSYKQRIKYVEDKVVDRIKELEDRVEELLSELKHLEQEVARLKKLVGER
  mature amino acid sequence for design gB#33
(is SEQ ID NO: 20 without the signal sequence)
SEQ ID NO: 21
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSYKPINEDLDEGIMVVYKRNIVAHTFKVRIYQKVLTFRRSYAYHRTTYLLGSNTEYVAPP
MWEIHHINSHGQCYSSYSRVIAGTVFVYYHRDSYENKTMQLMPDCYSNTHSTRYVTVKDQWHSRGSTRLYRETCN
VNCMVTITTARSKYPYHFFVTSTGDVVDISPFYNGTNRNASYFGENADKFFIFPNYTIVSDFGRPNAALETHRLV
AFLERADSVISWDIQDEKNVTCQLTFWEASERVIRSENEDSYHFISAKMTATFLTKKQEFNMSDSALDCVRDEAI
NKLQQIFNTSYNQTYEKYGNVSVFETTGGLIVFWQPIKQKSLVELERLANRSSLNLTHNSTKSSTDGNNATHLSN
MESVHNLVYAQLQFTYDTLRTYINQALAQIAEAWCVDQRRTLEIWKELSKINPSAILSAIYNRPIAARFMGDVLA
LSSCVTINQTSVKVLRDMNVKEDPGRCYSRPVVIFRFSNCSYVQYGQLGEDNEILLGNHRTEECQLPSLKIFIAG
NQAYEYVDYLFKRMIDLSSISTVDSMIDLNIDPLENTDERVLELYSQKELRSSNVFDLEEIMREFNSYKQRIKYV
EDKVVDRIKELEDRVEELLSELKHLEQEVARLKKLVGER
amino acid sequence for design gB#36 (predicted
signal sequence underlined)
SEQ ID NO: 22
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDCYSNTHSTR
YVTVKDQWHSRGSTRLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANCSYVQYGQLGEDNE
ILLGNHRTEECQLPSLKIFIAGNSAYEYVDYLFKRMIDLSSISTVDSMIALDIDPLENTDFRVLELYSQKELRSS
NVFDLEEIMREFNSYKQRVKYVEDKVVDRMKQLEDKVEELLSKNYHLENEVARLKKLVGER
mature amino acid sequence for design gB#36
(is SEQ ID NO: 22 without the signal sequence)
SEQ ID NO: 23
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDCYSNTHSTRYVTVKDQWHSRGSTRLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANCSYVQYGQLGEDNEILLGNHRTEECQLPSLKIFIAG
NSAYEYVDYLFKRMIDLSSISTVDSMIALDIDPLENTDERVLELYSQKELRSSNVEDLEEIMREFNSYKQRVKYV
EDKVVDRMKQLEDKVEELLSKNYHLENEVARLKKLVGER
amino acid sequence for design gB#37 (predicted
signal sequence underlined)
SEQ ID NO: 24
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGCDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFCTSTGDVVDISPFYNGTNRNASYFGENADKFFIF
PNYTIVSDFGRPNAALETHRLVAFLERADSVISWDIHDEKNVTCQLTFWEASERVIRSEAEDSYHFISAKMTATF
LTKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRAYINRALAQIAEAWCVDQRRTLEVWKELSKINP
SAILSAIYNKPIAARFMGDVLALSSCVTINQTSVKVLRDMNVKEDPGRCYSRPVVIFRFSNSSYVQYGQLGEDNE
ILLGNHRTEECQLPSLKIFIAGNQAYEYVDYLFKRMIDLSSISTVDSMISLNIDPLENTDERVLELYSQKELRSS
NVFDLEEIMREFNSYKQRIKYVEDKVVDRIKQLEDRVEELLSKLKHLEQEVARLKKLVGER
mature amino acid sequence for design gB#37
(is SEQ ID NO: 24 without the signal sequence)
SEQ ID NO: 25
VSSSSTRGTSATHSHHSSHTTSAAHSRSGSVSQRVTSSQTVSHGVNETIYNTTLKYGDVVGVNTTKYPYRVCSMA
QGCDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFCTSTGDVVDISPFYNGTNRNASYFGENADKFFIFPNYTIVSDFGRPNAALETHRLV
AFLERADSVISWDIHDEKNVTCQLTFWEASERVIRSEAEDSYHFISAKMTATFLTKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRAYINRALAQIAEAWCVDQRRTLEVWKELSKINPSAILSAIYNKPIAARFMGDVLA
LSSCVTINQTSVKVLRDMNVKEDPGRCYSRPVVIFRFSNSSYVQYGQLGEDNEILLGNHRTEECQLPSLKIFIAG
NQAYEYVDYLFKRMIDLSSISTVDSMISLNIDPLENTDFRVLELYSQKELRSSNVEDLEEIMREFNSYKQRIKYV
EDKVVDRIKQLEDRVEELLSKLKHLEQEVARLKKLVGER
amino acid sequence for design gB#40 (predicted
signal sequence underlined)
SEQ ID NO: 26
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGCDLIRFERNIVCTSMKPINEDLDEGIMVVYKRNIVAHTFKVRVYQKVLTER
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFCTSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDFRVLELYSQKELRSS
NVFDLEEIMREFNSYKQRVKYVEDKVVDRMKQLEDKVEELLSKNYHLENEVARLKKLVGER
mature amino acid sequence for design gB#40
(is SEQ ID NO: 26 without the signal sequence)
SEQ ID NO: 27
VSSSSTRGTSATHSHHSSHTTSAAHSRSGSVSQRVTSSQTVSHGVNETIYNTTLKYGDVVGVNTTKYPYRVCSMA
QGCDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFCTSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPLENTDFRVLELYSQKELRSSNVFDLEEIMREFNSYKQRVKYV
EDKVVDRMKQLEDKVEELLSKNYHLENEVARLKKLVGER
amino acid sequence for design gB#43 (predicted
signal sequence underlined)
SEQ ID NO: 28
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFVTSTCDVVDISPFYNGTNRNASYFGENADKFFIF
PNYTIVSDFGRPNSALETHRLVAFLERADSVISWDIQDEKNVTCQLTFWEASERVIRSEAEDSYHFISAKMTATF
LSKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRKYINRALAQIAEAWCVDQRRTLEVWKELSKINP
SAILSAIYNKPIAARFMGDVLALSSCVTINQTSVKVLRDMNVKEDPGRCYSRPVVIFRFSNSSYVQYGQLGEDNE
ILLGNHRTEECQLCSLKIFIAGNQAYEYVDYLFKRMIDLSSISTVDSMISLNIDPLENTDFRVLELYSQKELRSS
NVFDLEEIMREFNSYKQRIKYVEDKVVDRIKQLEDRVEELLSKLKHLEQEVARLKKLVGER
mature amino acid sequence for design gB#43
(is SEQ ID NO: 28 without the signal sequence)
SEQ ID NO: 29
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFVTSTCDVVDISPFYNGTNRNASYFGENADKFFIFPNYTIVSDFGRPNSALETHRLV
AFLERADSVISWDIQDEKNVTCQLTFWEASERVIRSEAEDSYHFISAKMTATFLSKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRKYINRALAQIAEAWCVDQRRTLEVWKELSKINPSAILSAIYNKPIAARFMGDVLA
LSSCVTINQTSVKVLRDMNVKEDPGRCYSRPVVIFRFSNSSYVQYGQLGEDNEILLGNHRTEECQLCSLKIFIAG
NQAYEYVDYLFKRMIDLSSISTVDSMISLNIDPLENTDFRVLELYSQKELRSSNVFDLEEIMREFNSYKQRIKYV
EDKVVDRIKQLEDRVEELLSKLKHLEQEVARLKKLVGER
amino acid sequence for design gB#44 (predicted
signal sequence underlined)
SEQ ID NO: 30
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATSTCDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLCSLKIFIAGNSAYEYVDYLFKRMIDLSSISTVDSMIALDIDPLENTDFRVLELYSQKELRSS
NVFDLEEIMREFNSYKQRVKYVEDKVVDRMKQLEDKVEELLSKNYHLENEVARLKKLVGER
mature amino acid sequence for design gB#44
(is SEQ ID NO: 30 without the signal sequence)
SEQ ID NO: 31
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATSTCDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANSSYVQYGQLGEDNEILLGNHRTEECQLCSLKIFIAG
NSAYEYVDYLFKRMIDLSSISTVDSMIALDIDPLENTDFRVLELYSQKELRSSNVFDLEEIMREFNSYKQRVKYV
EDKVVDRMKQLEDKVEELLSKNYHLENEVARLKKLVGER
amino acid sequence for design gB#47 (predicted
signal sequence underlined)
SEQ ID NO: 32
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATCTGDVVDISPFYNGTNRNASYFGENADKFFIF
PNYTIVSDFGRPNSALETHRLVAFLERADSVISWDIQDEKNVTCQLTFWEASERTIRSEAEDSYHFISAKMTATF
LSKKQEVNMSDSALDCVRDEAINKLQQIFNTSYNQTYEKYGNVSVFETTGGLIVFWQPIKQKSLVELERLANRSS
LNLTHNSTKSSTDGNNATHLSNMESVHNLVYAQLQFTYDTLRKYINRALAQIAEAWCVDQRRTLEVWKELSKINP
SAILSAIYNKPIAARFMGDVLGLASCVTINQTSVKVLRDMNVKESPGRCYSRPVVIFRFANSSYVQYGQLGEDNE
ILLGNHRTEECQLPSLKIFIAGNQAYEYVDYLFKRMIDLSSISTVDSMIALDIDPCENTDFRVLELYSQKELRSS
NVFDLEEIMREFNSYKQRVKYVEDKVVDRLKQLEDKVEELLSKLYHLEQEVARLKKLVGER
mature amino acid sequence for design gB#47
(is SEQ ID NO: 32 without the signal sequence)
SEQ ID NO: 33
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATCTGDVVDISPFYNGTNRNASYFGENADKFFIFPNYTIVSDFGRPNSALETHRLV
AFLERADSVISWDIQDEKNVTCQLTFWEASERTIRSEAEDSYHFISAKMTATFLSKKQEVNMSDSALDCVRDEAI
NKLQQIFNTSYNQTYEKYGNVSVFETTGGLIVFWQPIKQKSLVELERLANRSSLNLTHNSTKSSTDGNNATHLSN
MESVHNLVYAQLQFTYDTLRKYINRALAQIAEAWCVDQRRTLEVWKELSKINPSAILSAIYNKPIAARFMGDVLG
LASCVTINQTSVKVLRDMNVKESPGRCYSRPVVIFRFANSSYVQYGQLGEDNEILLGNHRTEECQLPSLKIFIAG
NQAYEYVDYLFKRMIDLSSISTVDSMIALDIDPCENTDFRVLELYSQKELRSSNVFDLEEIMREFNSYKQRVKYV
EDKVVDRLKQLEDKVEELLSKLYHLEQEVARLKKLVGER
amino acid sequence for design gB#48 (predicted
signal sequence underlined)
SEQ ID NO: 34
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATCTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPCENTDERVLELYSQKELRSS
NVFDLEEIMREFNSYKQRVKYVEDKVVDRMKQLEDKVEELLSKNYHLENEVARLKKLVGER
mature amino acid sequence for design gB#48
(is SEQ ID NO: 34 without the signal sequence)
SEQ ID NO: 35
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATCTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPCENTDFRVLELYSQKELRSSNVFDLEEIMREFNSYKQRVKYV
EDKVVDRMKQLEDKVEELLSKNYHLENEVARLKKLVGER
amino acid sequence for design gB#51 (predicted
signal sequence underlined)
SEQ ID NO: 36
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFVTSTGDVVDISPFYNGTNRNASYFGENADKFFIF
PNYTIVSDFGRPNSALETHRLVAFLERADSVISWDIHDEKNVTCQLTFWEASERVIRSEAEDSYHFISAKMTATF
LSKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRAYINRALAQIAEAWCVDQRRTLEVWKELSKICP
SAILSAIYNKPIAARFMGDVLALSSCVTINQTSVKVLRDMNVKEDPGRCYSRPVVIFRFANSSYVQYGQLGEDNE
ILLGNHRTEECQLPSLKIFIAGNQAYEYVDYLFKRMIDLSSISTVDSMIALDIDPLENTDERVLELYSQKELRSS
NVFDLEEICREFNSYKQRIKYVEDKVVDRIKQLEDKVEELLSKLKHLEQEVARLKKLVGER
mature amino acid sequence for design gB#51
(is SEQ ID NO: 36 without the signal sequence)
SEQ ID NO: 37
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFVTSTGDVVDISPFYNGTNRNASYFGENADKFFIFPNYTIVSDFGRPNSALETHRLV
AFLERADSVISWDIHDEKNVTCQLTFWEASERVIRSEAEDSYHFISAKMTATFLSKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRAYINRALAQIAEAWCVDQRRTLEVWKELSKICPSAILSAIYNKPIAARFMGDVLA
LSSCVTINQTSVKVLRDMNVKEDPGRCYSRPVVIFRFANSSYVQYGQLGEDNEILLGNHRTEECQLPSLKIFIAG
NQAYEYVDYLFKRMIDLSSISTVDSMIALDIDPLENTDFRVLELYSQKELRSSNVEDLEEICREFNSYKQRIKYV
EDKVVDRIKQLEDKVEELLSKLKHLEQEVARLKKLVGER
amino acid sequence for design gB#52 (predicted
signal sequence underlined)
SEQ ID NO: 38
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKICP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDERVLELYSQKELRSS
NVEDLEEICREFNSYKQRVKYVEDKVVDRMKQLEDKVEELLSKNYHLENEVARLKKLVGER
mature amino acid sequence for design gB#52
(is SEQ ID NO: 38 without the signal sequence)
SEQ ID NO: 39
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKICPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPLENTDFRVLELYSQKELRSSNVEDLEEICREFNSYKQRVKYV
EDKVVDRMKQLEDKVEELLSKNYHLENEVARLKKLVGER
amino acid sequence for design gB#55 (predicted
signal sequence underlined)
SEQ ID NO: 40
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDCYSNTHSTR
YVTVKDQWHSRGSTRLYRETCNLNCMVTITTARSKYPYHFFVTSTGDVVDISPFYNGTNRNASYFGENADKFFIF
PNYTIVSDFGRPNAALETHRLVAFLERADSVISWDIHDEKNVTCQLTFWEASERVIRSEAEDSYHFISAKMTATF
LTKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRKYINRALAQIAEAWCVDQRRTLEVWKELSKICP
SAILSAIYNKPIAARFMGDVLALSSCVTINQTSVKVLRDMNVKEDPGRCYSRPVVIFRFENCSYVQYGQLGEDNE
ILLGNHRTEECQLPSLKIFIAGNQAYEYVDYLFKRMIDLSSISTVDSMISLNIDPLENTDFRVLELYSQKELRSS
NVFDLEEICREFNSYKQRIRYVEDKVVDRIKQLEDRVDELLSKLKHLEQEVARLKKLVGER
mature amino acid sequence for design gB#55
(is SEQ ID NO: 40 without the signal sequence)
SEQ ID NO: 41
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDCYSNTHSTRYVTVKDQWHSRGSTRLYRETCN
LNCMVTITTARSKYPYHFFVTSTGDVVDISPFYNGTNRNASYFGENADKFFIFPNYTIVSDFGRPNAALETHRLV
AFLERADSVISWDIHDEKNVTCQLTFWEASERVIRSEAEDSYHFISAKMTATFLTKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRKYINRALAQIAEAWCVDQRRTLEVWKELSKICPSAILSAIYNKPIAARFMGDVLA
LSSCVTINQTSVKVLRDMNVKEDPGRCYSRPVVIFRFENCSYVQYGQLGEDNEILLGNHRTEECQLPSLKIFIAG
NQAYEYVDYLFKRMIDLSSISTVDSMISLNIDPLENTDERVLELYSQKELRSSNVEDLEEICREFNSYKQRIRYV
EDKVVDRIKQLEDRVDELLSKLKHLEQEVARLKKLVGER
amino acid sequence for design gB#56 (predicted
signal sequence underlined)
SEQ ID NO: 42
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDCYSNTHSTR
YVTVKDQWHSRGSTRLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKICP
SAILSAIYNKPIAARFMGDVLGLASCVTINQTSVKVLRDMNVKESPGRCYSRPVVIFNFANCSYVQYGQLGEDNE
ILLGNHRTEECQLPSLKIFIAGNSAYEYVDYLFKRMIDLSSISTVDSMIALDIDPLENTDERVLELYSQKELRSS
NVEDLEEICREFNSYKQRVKYVEDKVVDRMKQLEDKVEELLSKNYHLENEVARLKKLVGER
mature amino acid sequence for design gB#56
(is SEQ ID NO: 42 without the signal sequence)
SEQ ID NO: 43
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDCYSNTHSTRYVTVKDQWHSRGSTRLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKICPSAILSAIYNKPIAARFMGDVLG
LASCVTINQTSVKVLRDMNVKESPGRCYSRPVVIFNFANCSYVQYGQLGEDNEILLGNHRTEECQLPSLKIFIAG
NSAYEYVDYLFKRMIDLSSISTVDSMIALDIDPLENTDERVLELYSQKELRSSNVEDLEEICREFNSYKQRVKYV
EDKVVDRMKQLEDKVEELLSKNYHLENEVARLKKLVGER
amino acid sequence for design gB#59 (predicted
signal sequence underlined)
SEQ ID NO: 44
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGCDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFCTSTGDVVDISPFYNGTNRNASYFGENADKFFIF
PNYTIVSDFGRPNSALETHRLVAFLERADSVISWDIHDEKNVTCQLTFWEASERVIRSEAEDSYHFISAKMTATF
LSKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRKYINRALAQIAEAWCVDQRRTLEVWKELSKICP
SAILSAIYNKPIAARFMGDVLALSSCVTINQTSVKVLRDMNVKEDPGRCYSRPVVIFRFSNSSYVQYGQLGEDNE
ILLGNHRTEECQLPSLKIFIAGNQAYEYVDYLFKRMIDLSSISTVDSMISLNIDPLENTDERVLELYSQKELRSS
NVFDLEEICREFNSYKQRIKYVEDKVVDRIKQLEDRVEELLSKLKHLEQEVARLKKLVGER
mature amino acid sequence for design gB#59
(is SEQ ID NO: 44 without the signal sequence)
SEQ ID NO: 45
VSSSSTRGTSATHSHHSSHTTSAAHSRSGSVSQRVTSSQTVSHGVNETIYNTTLKYGDVVGVNTTKYPYRVCSMA
QGCDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFCTSTGDVVDISPFYNGTNRNASYFGENADKFFIFPNYTIVSDFGRPNSALETHRLV
AFLERADSVISWDIHDEKNVTCQLTFWEASERVIRSEAEDSYHFISAKMTATFLSKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRKYINRALAQIAEAWCVDQRRTLEVWKELSKICPSAILSAIYNKPIAARFMGDVLA
LSSCVTINQTSVKVLRDMNVKEDPGRCYSRPVVIFRFSNSSYVQYGQLGEDNEILLGNHRTEECQLPSLKIFIAG
NQAYEYVDYLFKRMIDLSSISTVDSMISLNIDPLENTDERVLELYSQKELRSSNVEDLEEICREFNSYKQRIKYV
EDKVVDRIKQLEDRVEELLSKLKHLEQEVARLKKLVGER
amino acid sequence for design gB#60 (predicted
signal sequence underlined)
SEQ ID NO: 46
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGCDLIRFERNIVCTSMKPINEDLDEGIMVVYKRNIVAHTFKVRVYQKVLTER
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFCTSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKICP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDERVLELYSQKELRSS
NVFDLEEICREFNSYKQRVKYVEDKVVDRMKQLEDKVEELLSKNYHLENEVARLKKLVGER
mature amino acid sequence for design gB#60
(is SEQ ID NO: 46 without the signal sequence)
SEQ ID NO: 47
VSSSSTRGTSATHSHHSSHTTSAAHSRSGSVSQRVTSSQTVSHGVNETIYNTTLKYGDVVGVNTTKYPYRVCSMA
QGCDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFCTSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKICPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPLENTDFRVLELYSQKELRSSNVFDLEEICREFNSYKQRVKYV
EDKVVDRMKQLEDKVEELLSKNYHLENEVARLKKLVGER
amino acid sequence for design gB#63 (predicted
signal sequence underlined)
SEQ ID NO: 48
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFVTSTCDVVDISPFYNGTNRNASYFGENADKFFIF
PNYTIVSDFGRPNSALETHRLVAFLERADSVISWDIHDEKNVTCQLTFWEASERVIRSEAEDSYHFISAKMTATF
LSKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRKYINRALAQIAEAWCVDQRRTLEVWKELSKICP
SAILSAIYNKPIAARFMGDVLALSSCVTINQTSVKVLRDMNVKEDPGRCYSRPVVIFRFENSSYVQYGQLGEDNE
ILLGNHRTEECQLCSLKIFIAGNQAYEYVDYLFKRMIDLSSISTVDSMISLNIDPLENTDERVLELYSQKELRSS
NVEDLEEICREFNSYKQRIKYVEDKVVDRIKQLEDRVDELLSKLKHLEQEVARLKKLVGER
mature amino acid sequence for design gB#63
(is SEQ ID NO: 48 without the signal sequence)
SEQ ID NO: 49
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFVISTCDVVDISPFYNGTNRNASYFGENADKFFIFPNYTIVSDFGRPNSALETHRLV
AFLERADSVISWDIHDEKNVTCQLTFWEASERVIRSEAEDSYHFISAKMTATFLSKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRKYINRALAQIAEAWCVDQRRTLEVWKELSKICPSAILSAIYNKPIAARFMGDVLA
LSSCVTINQTSVKVLRDMNVKEDPGRCYSRPVVIFRFENSSYVQYGQLGEDNEILLGNHRTEECQLCSLKIFIAG
NQAYEYVDYLFKRMIDLSSISTVDSMISLNIDPLENTDERVLELYSQKELRSSNVFDLEEICREFNSYKQRIKYV
EDKVVDRIKQLEDRVDELLSKLKHLEQEVARLKKLVGER
amino acid sequence for design gB#64 (predicted
signal sequence underlined)
SEQ ID NO: 50
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTER
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATSTCDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKICP
SAILSAIYNKPIAARFMGDVLGLASCVTINQTSVKVLRDMNVKESPGRCYSRPVVIFNFANSSYVQYGQLGEDNE
ILLGNHRTEECQLCSLKIFIAGNSAYEYVDYLFKRMIDLSSISTVDSMIALDIDPLENTDERVLELYSQKELRSS
NVEDLEEICREFNSYKQRVKYVEDKVVDRMKQLEDKVEELLSKNYHLENEVARLKKLVGER
mature amino acid sequence for design gB#64
(is SEQ ID NO: 50 without the signal sequence)
SEQ ID NO: 51
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATSTCDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKICPSAILSAIYNKPIAARFMGDVLG
LASCVTINQTSVKVLRDMNVKESPGRCYSRPVVIFNFANSSYVQYGQLGEDNEILLGNHRTEECQLCSLKIFIAG
NSAYEYVDYLFKRMIDLSSISTVDSMIALDIDPLENTDERVLELYSQKELRSSNVFDLEEICREFNSYKQRVKYV
EDKVVDRMKQLEDKVEELLSKNYHLENEVARLKKLVGER
amino acid sequence for design gB#67 (predicted
signal sequence underlined)
SEQ ID NO: 52
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGGDLFRFERNIVCTSMKPINEDLDEGIMVVYKRNIVAHTFKVRIYQKVLTFR
RSYAYHRTTYLLGSNTEYVAPPMWEIHHINSHGQCYSSYSRVIAGTVFVAYHRDSYENKTMQLMPDDYSNTHSTR
YVTVKDQWHSRGSTRLYRETCNLNCMVTITTARSKYPYHFFVTCTGDVVDISPFYNGTNRNASYFGENADKFFIF
PNYTIVSDFGRPNSALETHRLVAFLERADSVISWDIHDEKNVTCQLTFWEASERVIRSEAEDSYHFISAKMTATF
LSKKQEVNMSDSALDCVRDEAINKLQQIFNTSYNQTYEKYGNVSVFETTGGLFVFWQPIKQKSLVELERLANRSS
LNLTHNSTKSSTDGNNATHLSNMESVHNLVYAQLQFTYDTLRAYINRALAQIAEAWCVDQRRTLEVWKELSKICP
SAILSAIYNKPIAARFMGDVLALSSCVTINQTSVKVLRDMNVKEDPGRCYSRPVVIFRFENSSYVQYGQLGEDNE
ILLGNHRTEECQLPSLKIFIAGNQAYEYVDYLFKRMIDLSSISTVDSMISLNIDPCENTDERVLELYSQKELRSS
NVFDLEEICREFNSYKQRIKYVEDKVVDRIKQLEDRVEELLSKLKHLEQEVARLKKLVGER
mature amino acid sequence for design gB#67
(is SEQ ID NO: 52 without the signal sequence)
SEQ ID NO: 53
VSSSSTRGTSATHSHHSSHTTSAAHSRSGSVSQRVTSSQTVSHGVNETIYNTTLKYGDVVGVNTTKYPYRVCSMA
QGGDLFRFERNIVCTSMKPINEDLDEGIMVVYKRNIVAHTFKVRIYQKVLTFRRSYAYHRTTYLLGSNTEYVAPP
MWEIHHINSHGQCYSSYSRVIAGTVFVAYHRDSYENKTMQLMPDDYSNTHSTRYVTVKDQWHSRGSTRLYRETCN
LNCMVTITTARSKYPYHFFVTCTGDVVDISPFYNGTNRNASYFGENADKFFIFPNYTIVSDFGRPNSALETHRLV
AFLERADSVISWDIHDEKNVTCQLTFWEASERVIRSEAEDSYHFISAKMTATFLSKKQEVNMSDSALDCVRDEAI
NKLQQIFNTSYNQTYEKYGNVSVFETTGGLFVFWQPIKQKSLVELERLANRSSLNLTHNSTKSSTDGNNATHLSN
MESVHNLVYAQLQFTYDTLRAYINRALAQIAEAWCVDQRRTLEVWKELSKICPSAILSAIYNKPIAARFMGDVLA
LSSCVTINQTSVKVLRDMNVKEDPGRCYSRPVVIFRFENSSYVQYGQLGEDNEILLGNHRTEECQLPSLKIFIAG
NQAYEYVDYLFKRMIDLSSISTVDSMISLNIDPCENTDERVLELYSQKELRSSNVEDLEEICREENSYKQRIKYV
EDKVVDRIKQLEDRVEELLSKLKHLEQEVARLKKLVGER
amino acid sequence for design gB#68 (predicted
signal sequence underlined)
SEQ ID NO: 54
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYHRTTYLLGSNTEYVAPPMWEIHHINSHSQCYSSYSRVIAGTVFVAYHRDSYENKTMQLMPDDYSNTHSTR
YVTVKDQWHSRGSTRLYRETCNLNCMVTITTARSKYPYHFFATCTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKICP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPCENTDERVLELYSQKELRSS
NVEDLEEICREFNSYKQRVKYVEDKVVDRMKQLEDKVEELLSKNYHLENEVARLKKLVGER
mature amino acid sequence for design gB#68
(is SEQ ID NO: 54 without the signal sequence)
SEQ ID NO: 55
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYHRTTYLLGSNTEYVAPP
MWEIHHINSHSQCYSSYSRVIAGTVFVAYHRDSYENKTMQLMPDDYSNTHSTRYVTVKDQWHSRGSTRLYRETCN
LNCMVTITTARSKYPYHFFATCTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKICPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPCENTDERVLELYSQKELRSSNVEDLEEICREENSYKQRVKYV
EDKVVDRMKQLEDKVEELLSKNYHLENEVARLKKLVGER
amino acid sequence for design gB#70 (predicted
signal sequence underlined)
SEQ ID NO: 56
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMATGGDLVRFERNIVCTSMKPINEDLDEGIMVVYKRNIVAHTFKVRVYQKVLTFR
RSYAYIHTTYLLGSNTEYVAPPMWEIHHINKHGQCYSSYSRVIAGTVFVYYHRDSYENKTMQLMPDDYSNTHSTR
YVTVKDQWHSRGSCGLYRETCTLNCMVTIVTARSKYPYHFFVLSTGDIVDISPFYDGTNRNASYFGENADKFFIF
PNYTIVSDFGRPNAALETHRLVAFLERADSVISWDIQDEKNVTCQLTFWDASERTIRTETEDSYHFISAKMTATF
IAKKQEVNESDSALDCVRDEAINKLQQIFNTSYNQTYEKYGNVSVFETTGGLIVFWQPIKQKSLVELERLANRSS
LNLTHNSTKSSTDGNNATHLSNMESVHNLVYAQLQFTYDTLRKYINRALAQIAEAWCVDQRRTLEIWKELSKINP
SAILSAIYNRPIAARWLGDVLGISSCVTINQTSVKVLRDMNVKEDPGRCYSRPVVIFQFANSSYVFYGQLGEDNE
ILLGNHRTEECQLPSLKIFITGNSAYEYVDYLFKRMIDLSSISTVDSMIDLDIDPLENTDERLLELYSQKELRSS
NVFDLEEIMREFNSYKQRVKEIEDKVVDNLPPYLQGLDSLMSDLGTVGKMEELFKKHKIVAVLRANSVEEAKEKA
LALFEGGVHLIEITFTVPDADKVIKELSFLKEKGAIIGAGTVTSVEQCRKAICSGAEFIVSPHLDEEISRFCKEK
GVFYMPGVMTPTELVKALKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#70
(is SEQ ID NO: 56 without the signal sequence)
SEQ ID NO: 57
VSSSSTRGTSATHSHHSSHTTSAAHSRSGSVSQRVTSSQTVSHGVNETIYNTTLKYGDVVGVNTTKYPYRVCSMA
TGGDLVRFERNIVCTSMKPINEDLDEGIMVVYKRNIVAHTFKVRVYQKVLTFRRSYAYIHTTYLLGSNTEYVAPP
MWEIHHINKHGQCYSSYSRVIAGTVFVYYHRDSYENKTMQLMPDDYSNTHSTRYVTVKDQWHSRGSCGLYRETCT
LNCMVTIVTARSKYPYHFFVLSTGDIVDISPFYDGTNRNASYFGENADKFFIFPNYTIVSDFGRPNAALETHRLV
AFLERADSVISWDIQDEKNVTCQLTFWDASERTIRTETEDSYHFISAKMTATFIAKKQEVNESDSALDCVRDEAI
NKLQQIFNTSYNQTYEKYGNVSVFETTGGLIVFWQPIKQKSLVELERLANRSSLNLTHNSTKSSTDGNNATHLSN
MESVHNLVYAQLQFTYDTLRKYINRALAQIAEAWCVDQRRTLEIWKELSKINPSAILSAIYNRPIAARWLGDVLG
ISSCVTINQTSVKVLRDMNVKEDPGRCYSRPVVIFQFANSSYVFYGQLGEDNEILLGNHRTEECQLPSLKIFITG
NSAYEYVDYLFKRMIDLSSISTVDSMIDLDIDPLENTDERLLELYSQKELRSSNVFDLEEIMREFNSYKQRVKEI
EDKVVDNLPPYLQGLDSLMSDLGTVGKMEELFKKHKIVAVLRANSVEEAKEKALALFEGGVHLIEITFTVPDADK
VIKELSFLKEKGAIIGAGTVTSVEQCRKAICSGAEFIVSPHLDEEISRFCKEKGVFYMPGVMTPTELVKALKLGH
TILKLFPGEVVGPQFVKAMKGPFPNVKFVPTGGVNLDNVCEWFKAGVLAVGVGSALVKGTPDEVREKAKAFVEKI
RGC
amino acid sequence for design gB#72 (predicted
signal sequence underlined)
SEQ ID NO: 58
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYIHTTYLLGSNTEYVAPPMWEIHHINSHSQCYSSYSRVIAGTVFVAYHRDSYENKTMQLMPDDYSNTHSTR
YVTVKDQWHSRGSCWLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDERVLELYSQKELRSS
NVFDLEEIMREFNSYKQRVKYVEDKVVDPLPPYLKGLDDLMSGLGAAGKMEELFKKHKIVAVLRANSVEEAKEKA
LAVFEGGVHLIEITFTVPDADTVIKELSFLKEKGAIIGAGTVTSVEQCRKAVCSGAEFIVSPHLDEEISQFCKEK
GVFYMPGVMTPTELVKAMKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#72
(is SEQ ID NO: 58 without the signal sequence)
SEQ ID NO: 59
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYIHTTYLLGSNTEYVAPP
MWEIHHINSHSQCYSSYSRVIAGTVFVAYHRDSYENKTMQLMPDDYSNTHSTRYVTVKDQWHSRGSCWLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPLENTDFRVLELYSQKELRSSNVFDLEEIMREFNSYKQRVKYV
EDKVVDPLPPYLKGLDDLMSGLGAAGKMEELFKKHKIVAVLRANSVEEAKEKALAVFEGGVHLIEITFTVPDADT
VIKELSFLKEKGAIIGAGTVTSVEQCRKAVCSGAEFIVSPHLDEEISQFCKEKGVFYMPGVMTPTELVKAMKLGH
TILKLFPGEVVGPQFVKAMKGPFPNVKFVPTGGVNLDNVCEWFKAGVLAVGVGSALVKGTPDEVREKAKAFVEKI
RGC
amino acid sequence for design gB#75 (predicted
signal sequence underlined)
SEQ ID NO: 60
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMATGGDLVRFERNIVCTSMKPINEDLDEGIMVVYKRNIVAHTFKVRVYQKVLTFR
RSYAYCHTTYLLGSNTEYVAPPMWEIHHINKHGQCYSSYSRVIAGTVFVYYHRDSYENKTMQLMPDDYSNTHSTR
YVTVKDQWHSRGSTGLYRETCTLNCMVTITEARSKYPYHFFVLSTGDIVDISPFYDGTNRNASYFGENADKFFIF
PNYTIVSDFGRPNAALETHRLVAFLERADSVISWDIQDEKNVTCQLTFWDASERTIRSETEDSYHFISAKMTATF
VAKKQEVNPSDSALDCVRDEAINKLQQIFNTSYNQTYEKYGNVSVFETTGGLIVFWQPIKQKSLVELERLANRSS
LNLTHNSTKSSTDGNNATHLSNMESVHNLVYAQLQFTYDTLRKYINRALAQIAEAWCVDQRRTLEIWKELSKINP
SAILSAIYNRPIAARWLGDVLGISSCVTINQTSVKVLRDMNVKEDPGRCYSRPVVIFQFANSSYVFYGQLGEDNE
ILLGNHRTEECQLPSLKIFITGNSAYEYVDYLFKRMIDLSSISTVDSMIDLDIDPLENTDERLLELYSQKELRSS
NVFDLEEIMREFNSYKQRVKEVEDKVVDNLPPYLQGLDSLMDDLGSVGKMEELFKKHKIVAVLRANSVEEAKEKA
LALFEGGVHLIEITFTVPDADKVIKELSFLKEKGAIIGAGTVTSVEQCRKACESGAEFIVSPHLDEEISRFCKEK
GVFYMPGVMTPTELVKALKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#75
(is SEQ ID NO: 60 without the signal sequence)
SEQ ID NO: 61
VSSSSTRGTSATHSHHSSHTTSAAHSRSGSVSQRVTSSQTVSHGVNETIYNTTLKYGDVVGVNTTKYPYRVCSMA
TGGDLVRFERNIVCTSMKPINEDLDEGIMVVYKRNIVAHTFKVRVYQKVLTFRRSYAYCHTTYLLGSNTEYVAPP
MWEIHHINKHGQCYSSYSRVIAGTVFVYYHRDSYENKTMQLMPDDYSNTHSTRYVTVKDQWHSRGSTGLYRETCT
LNCMVTITEARSKYPYHFFVLSTGDIVDISPFYDGTNRNASYFGENADKFFIFPNYTIVSDFGRPNAALETHRLV
AFLERADSVISWDIQDEKNVTCQLTFWDASERTIRSETEDSYHFISAKMTATFVAKKQEVNPSDSALDCVRDEAI
NKLQQIFNTSYNQTYEKYGNVSVFETTGGLIVFWQPIKQKSLVELERLANRSSLNLTHNSTKSSTDGNNATHLSN
MESVHNLVYAQLQFTYDTLRKYINRALAQIAEAWCVDQRRTLEIWKELSKINPSAILSAIYNRPIAARWLGDVLG
ISSCVTINQTSVKVLRDMNVKEDPGRCYSRPVVIFQFANSSYVFYGQLGEDNEILLGNHRTEECQLPSLKIFITG
NSAYEYVDYLFKRMIDLSSISTVDSMIDLDIDPLENTDERLLELYSQKELRSSNVFDLEEIMREFNSYKQRVKEV
EDKVVDNLPPYLQGLDSLMDDLGSVGKMEELFKKHKIVAVLRANSVEEAKEKALALFEGGVHLIEITFTVPDADK
VIKELSFLKEKGAIIGAGTVTSVEQCRKACESGAEFIVSPHLDEEISRFCKEKGVFYMPGVMTPTELVKALKLGH
TILKLFPGEVVGPQFVKAMKGPFPNVKFVPTGGVNLDNVCEWFKAGVLAVGVGSALVKGTPDEVREKAKAFVEKI
RGC
amino acid sequence for design gB#76 (predicted
signal sequence underlined)
SEQ ID NO: 62
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYCHTTYLLGSNTEYVAPPMWEIHHINSHSQCYSSYSRVIAGTVFVAYHRDSYENKTMQLMPDDYSNTHSTR
YVTVKDQWHSRGSTWLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDERVLELYSQKELRSS
NVFDLEEIMREFNSYKQRVKYVEDKVVDPLPPYLKGLDDLMSGLGAAGKMEELFKKHKIVAVLRANSVEEAKEKA
LAVFEGGVHLIEITFTVPDADTVIKELSFLKEKGAIIGAGTVTSVEQCRKACESGAEFIVSPHLDEEISQFCKEK
GVFYMPGVMTPTELVKAMKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#76
(is SEQ ID NO: 62 without the signal sequence)
SEQ ID NO: 63
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYCHTTYLLGSNTEYVAPP
MWEIHHINSHSQCYSSYSRVIAGTVFVAYHRDSYENKTMQLMPDDYSNTHSTRYVTVKDQWHSRGSTWLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANSSYVQYGQLGEDNEILLGNHRTEECQLPSLKIFIAG
NSAYEYVDYLFKRMIDLSSISTVDSMIALDIDPLENTDERVLELYSQKELRSSNVEDLEEIMREFNSYKQRVKYV
EDKVVDPLPPYLKGLDDLMSGLGAAGKMEELFKKHKIVAVLRANSVEEAKEKALAVFEGGVHLIEITFTVPDADT
VIKELSFLKEKGAIIGAGTVTSVEQCRKACESGAEFIVSPHLDEEISQFCKEKGVFYMPGVMTPTELVKAMKLGH
TILKLFPGEVVGPQFVKAMKGPFPNVKFVPTGGVNLDNVCEWFKAGVLAVGVGSALVKGTPDEVREKAKAFVEKI
RGC
amino acid sequence for design gB#79 (predicted
signal sequence underlined)
SEQ ID NO: 64
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMANGGDLIRFERNIVCTSMKPINEDLDEGIMVVYKRNIVAHTFKVRVYQKVLTFR
RSYAYIHTTYLLGSNTEYVAPPMWEIHHINRHGQCYSSYSRVIAGTVFVAYHRDSYENKTMQLMPDCYSNTHSTR
YVTVKDQWHSRGSTGLYRETCNLNCMVTIVTARSKYPYHFFALSTGDVVDISPFYDGTNRNASYFGENADKFFIF
PNYTIVSDFGRPNSALETHRLVAFLERADSVISWDIQDEKNVTCQLTFWDASERTIRSETEDSYHFISAKMTATF
LSKKQEVNMSDSALDCVRDEAINKLQQIFNTSYNQTYEKYGNVSVFETTGGLVVFWQPIKQKSLVELERLANRSS
LNLTHNSTKSSTDGNNATHLSNMESVHNLVYAQLQFTYDTLREYINRALAQIAEAWCVDQRRTLEVWKELSKINP
SAILSAIYNKPIAARFMGDVLGISSCVTINQTSVKVLRDMNVKESPGRCYSRPVVIFNFANCSYVFYGQLGEDNE
ILLGNHRTEECQLPSLKIFIAGNSAYEYVDYLFKRMIDLSSISTVDSMIDLDIDPLENTDERVLELYSQKELRSS
NVFDLEEIMREFNSYKQRVKYVEDKVVDNLPPYLKGLDSLMSDLGDVGKMEELFKKHKIVAVLRANSVEEAKEKA
LALFEGGVHLIEITFTVPDADKVIKELSFLKEKGAIIGAGTVTSVEQCRKAVESGAEFIVSPHLDEEISQFCKEK
GVFYMPGVMTPTELVKALKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#79
(is SEQ ID NO: 64 without the signal sequence)
SEQ ID NO: 65
VSSSSTRGTSATHSHHSSHTTSAAHSRSGSVSQRVTSSQTVSHGVNETIYNTTLKYGDVVGVNTTKYPYRVCSMA
NGGDLIRFERNIVCTSMKPINEDLDEGIMVVYKRNIVAHTFKVRVYQKVLTFRRSYAYIHTTYLLGSNTEYVAPP
MWEIHHINRHGQCYSSYSRVIAGTVFVAYHRDSYENKTMQLMPDCYSNTHSTRYVTVKDQWHSRGSTGLYRETCN
LNCMVTIVTARSKYPYHFFALSTGDVVDISPFYDGTNRNASYFGENADKFFIFPNYTIVSDFGRPNSALETHRLV
AFLERADSVISWDIQDEKNVTCQLTFWDASERTIRSETEDSYHFISAKMTATFLSKKQEVNMSDSALDCVRDEAI
NKLQQIFNTSYNQTYEKYGNVSVFETTGGLVVFWQPIKQKSLVELERLANRSSLNLTHNSTKSSTDGNNATHLSN
MESVHNLVYAQLQFTYDTLREYINRALAQIAEAWCVDQRRTLEVWKELSKINPSAILSAIYNKPIAARFMGDVLG
ISSCVTINQTSVKVLRDMNVKESPGRCYSRPVVIFNFANCSYVFYGQLGEDNEILLGNHRTEECQLPSLKIFIAG
NSAYEYVDYLFKRMIDLSSISTVDSMIDLDIDPLENTDFRVLELYSQKELRSSNVFDLEEIMREFNSYKQRVKYV
EDKVVDNLPPYLKGLDSLMSDLGDVGKMEELFKKHKIVAVLRANSVEEAKEKALALFEGGVHLIEITFTVPDADK
VIKELSFLKEKGAIIGAGTVTSVEQCRKAVESGAEFIVSPHLDEEISQFCKEKGVFYMPGVMTPTELVKALKLGH
TILKLFPGEVVGPQFVKAMKGPFPNVKFVPTGGVNLDNVCEWFKAGVLAVGVGSALVKGTPDEVREKAKAFVEKI
RGC
amino acid sequence for design gB#80 (predicted
signal sequence underlined)
SEQ ID NO: 66
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYIHTTYLLGSNTEYVAPPMWEIHHINSHSQCYSSYSRVIAGTVFVAYHRDSYENKTMQLMPDCYSNTHSTR
YVTVKDQWHSRGSTWLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNSTKSSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANCSYVQYGQLGEDNE
ILLGNHRTEECQLPSLKIFIAGNSAYEYVDYLFKRMIDLSSISTVDSMIALDIDPLENTDERVLELYSQKELRSS
NVFDLEEIMREFNSYKQRVKYVEDKVVDPLPPYLKGLDDLMSGLGAAGKMEELFKKHKIVAVLRANSVEEAKEKA
LAVFEGGVHLIEITFTVPDADTVIKELSFLKEKGAIIGAGTVTSVEQCRKAVESGAEFIVSPHLDEEISQFCKEK
GVFYMPGVMTPTELVKAMKLGHTILKLFPGEVVGPQFVKAMKGPFPNVKFVPTGGVNLDNVCEWFKAGVLAVGVG
SALVKGTPDEVREKAKAFVEKIRGC
mature amino acid sequence for design gB#80
(is SEQ ID NO: 66 without the signal sequence)
SEQ ID NO: 67
VSSSSTRGTSATHSHHSSHTTSAAHSRSGSVSQRVTSSQTVSHGVNETIYNTTLKYGDVVGVNTTKYPYRVCSMA
QGTDLIRFERNIVCTSMKPINEDLDEGIMVVYKRNIVAHTFKVRVYQKVLTFRRSYAYIHTTYLLGSNTEYVAPP
MWEIHHINSHSQCYSSYSRVIAGTVFVAYHRDSYENKTMQLMPDCYSNTHSTRYVTVKDQWHSRGSTWLYRETCN
LNCMVTITTARSKYPYHFFATSTGDVVDISPFYNGTNRNASYFGENADKFFIFPNYTIVSDFGRPNSALETHRLV
AFLERADSVISWDIQDEKNVTCQLTFWEASERTIRSEAEDSYHFSSAKMTATFLSKKQEVNMSDSALDCVRDEAI
NKLQQIFNTSYNQTYEKYGNVSVFETTGGLVVFWQGIKQKSLVELERLANRSSLNLTHNSTKSSTDGNNATHLSN
MESVHNLVYAQLQFTYDTLRGYINRALAQIAEAWCVDQRRTLEVFKELSKINPSAILSAIYNKPIAARFMGDVLG
LASCVTINQTSVKVLRDMNVKESPGRCYSRPVVIFNFANCSYVQYGQLGEDNEILLGNHRTEECQLPSLKIFIAG
NSAYEYVDYLFKRMIDLSSISTVDSMIALDIDPLENTDERVLELYSQKELRSSNVEDLEEIMREFNSYKQRVKYV
EDKVVDPLPPYLKGLDDLMSGLGAAGKMEELFKKHKIVAVLRANSVEEAKEKALAVFEGGVHLIEITFTVPDADT
VIKELSFLKEKGAIIGAGTVTSVEQCRKAVESGAEFIVSPHLDEEISQFCKEKGVFYMPGVMTPTELVKAMKLGH
TILKLFPGEVVGPQFVKAMKGPFPNVKFVPTGGVNLDNVCEWFKAGVLAVGVGSALVKGTPDEVREKAKAFVEKI
RGC
```

1. An HCMV gB protein with an amino acid sequence comprising:
(a) at least one, at least two, at least three, at least four, at least five, or at least six recombinant Cysteine (C) residues; and
(b) a heterologous trimerization domain operably linked c-terminal to the residue corresponding to any one of 698, 699, 700, 701, 702, 703, 704, 705, 706, 707, or 708 of SEQ ID NO: 1.

2. The HCMV gB protein of claim 1, wherein the amino acid sequence comprises:
(c) a Domain I amino acid sequence that is at least 190 total residues in length, a Domain II amino acid sequence that is at least 83 total residues in length, a Domain III amino acid sequence that is at least 76 total residues in length, a Domain IV amino acid sequence that is at least 87 total residues in length, and a Domain V amino acid sequence that is at least 30 total residues in length.

3. The HCMV gB protein of claim 1, wherein the amino acid sequence comprises: except for the at least one, at least two, at least three, at least four, at least five, or at least six recombinant Cysteine (C) residues, residues that correspond to residues 89-648 of SEQ ID NO: 1 and at least 30 consecutive residues from 649-698 of SEQ ID NO: 1.

4. (canceled)

5. The HCMV gB protein of claim 1, wherein the amino acid sequence comprises:
i. a recombinant cysteine residue at residue 239, numbered according to SEQ ID NO: 1;
ii. a recombinant cysteine residue at residue 156, numbered according to SEQ ID NO: 1;
iii. a recombinant cysteine residue at residues 217 and 587; numbered according to SEQ ID NO: 1;
iv. a recombinant cysteine residue at residues 524 and 684; numbered according to SEQ ID NO: 1;
v. a recombinant cysteine residue at residues 100 and 267; numbered according to SEQ ID NO: 1;
vi. a recombinant cysteine residue at residues 269 and 656; numbered according to SEQ ID NO: 1;
vii. a recombinant cysteine residue at residues 251 and 614; numbered according to SEQ ID NO: 1;
viii. a recombinant cysteine residue at residues 100, 251, and 614; numbered according to SEQ ID NO: 1;
ix. a recombinant cysteine residue at residues 100, 269, and 656; numbered according to SEQ ID NO: 1;
x. a recombinant cysteine residue at residues 269, 524, 656, and 684; numbered according to SEQ ID NO: 1;
xi. a recombinant cysteine residue at residues 100, 267, 524, and 684; numbered according to SEQ ID NO: 1;
xii. a recombinant cysteine residue at residues 251, 524, 614, and 684; numbered according to SEQ ID NO: 1;
xiii. a recombinant cysteine residue at residues 217, 524, 587, and 684; numbered according to SEQ ID NO: 1;
xiv. a recombinant cysteine residue at residues 100, 217, 524, 587, and 684; numbered according to SEQ ID NO: 1;
xv. a recombinant cysteine residue at residues 100, 251, 524, 614, and 684; numbered according to SEQ ID NO: 1; or
xvi. a recombinant cysteine residue at residues 217, 269, 524, 587, 656, and 684; numbered according to SEQ ID NO: 1.

6. The HCMV gB protein of claim 1, wherein the heterologous trimerization domain is a GCN4 or aldolase sequence.

7. The HCMV gB protein of claim 6, wherein the heterologous trimerization domain is a GCN4 sequence selected from the group consisting of SEQ ID NOs: 2, 12, 13, 14, 15, 16, and 17.

8. The HCMV gB protein of claim 6, wherein the heterologous trimerization domain is an aldolase sequence selected from the group consisting of SEQ ID NOs: 18 and 19.

9. The HCMV gB protein of claim 1, wherein the amino acid sequence comprises an amino acid linker between the residue corresponding to 698, 699, 700, 701, 702, or 703, numbered according to SEQ ID NO: 1, and the heterologous trimerization domain.

10. The HCMV gB protein of claim 1, wherein the amino acid sequence comprises:
(i) a substitution of one or more furin cleavage site amino acid;
(ii) a substitution of one or more fusion loop amino acid; or
(iii) both (i) and (ii).

11. The HCMV gB protein of claim 1, wherein the amino acid sequence comprises a purification tag.

12. The HCMV gB protein of claim 1, wherein the amino acid sequence does not comprise a recombinant proline residue, comprises only one recombinant proline residue at the position corresponding to residue 433 of SEQ ID NO: 1, or comprises only two recombinant proline residues at the positions corresponding to residues 433 and 384 of SEQ ID NO: 1.

13. The HCMV gB protein of claim 1, that is in pre-fusogenic conformation.

14. (canceled)

15. The HCMV gB protein of claim 1, comprising an amino acid sequence that is at least 90%, 91%, 92%, 93%, 94%, 95%, 96%, 97%, 98%, 99%, or 100% identical to the amino acid sequence set out in any one of SEQ ID NOs: 3, 4, 11, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67.

16. (canceled)

17. An isolated nucleic acid comprising a polynucleotide sequence encoding the HCMV gB protein of claim 1.

18. An immunogenic composition comprising the HCMV gB protein of claim 1.

19. The immunogenic composition of claim 18, further comprising at least one additional antigenic protein, or nucleic acid molecule encoding the at least one additional antigenic protein, wherein the at least one additional antigenic protein is gO, gH, gL, pUL128, pUL130, pUL131, pp65, IE1, an antigenic fragment thereof, or a combination thereof; optionally gH or a gH fragment, gL, pUL128, pUL130, and pUL131.

20. (canceled)

21. The immunogenic composition of claim 18, further comprising an adjuvant, wherein the adjuvant comprises an aluminum salt, a TLR7 agonist, TLR4 agonist, MPL, 3D-MPL, saponin, or an oil-in-water emulsion.

22. (canceled)

23. A recombinant vector comprising the nucleic acid of claim 17.

24. An isolated host cell comprising the nucleic acid of claim 17.

25. (canceled)

26. (canceled)

27. (canceled)

28. (canceled)

29. (canceled)

30. (canceled)

31. (canceled)

32. (canceled)

33. A method of inducing an immune response against HCMV in a subject, comprising administering to the subject an immunologically effective amount of the HCMV gB protein of claim 1.


SEQUENCE LISTING (67 sequences)

SEQ ID NO: 1 - protein, 907 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 2 - protein, 33 aa, synthetic construct
RMKQLEDKVEELLSKNYHLENEVARLKKLVGER

SEQ ID NO: 3 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPCENTDFRVLELYSQKELRSSNVFDLEEICREFNSYKQRVKYVEDKVVDRMKQLEDKVEELLSKNYHLENEVARLKKLVGER

SEQ ID NO: 4 - protein, 750 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPCENTDFRVLELYSQKELRSSNVFDLEEICREFNSYKQRVKYVEDKVVDRMKQLEDKVEELLSKNYHLENEVARLKKLVGERENLYFQGGHHHHHH

SEQ ID NO: 5 - protein, 14 aa, synthetic construct
ENLYFQGGHHHHHH

SEQ ID NO: 6 - protein, 906 aa, synthetic construct
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 7 - protein, 8 aa, synthetic construct
DYKDDDDK

SEQ ID NO: 8 - protein, 9 aa, synthetic construct
AWRHPQFGG

SEQ ID NO: 9 - protein, 8 aa, synthetic construct
WSHPQFEK

SEQ ID NO: 10 - protein, 28 aa, synthetic construct
WSHPQFEKGGGSGGGSGGGSWSHPQFEK

SEQ ID NO: 11 - protein, 749 aa, synthetic construct
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYHRTTYLLGSNTEYVAPPMWEIHHINKFAQCYSSYSRVIGGTVFVAYHRDSYENKTMQLIPDCYSNTHSTRYVTVKDQWHSRGSTRLYRETCNLNCMLTITTARSKYPYHFFATCTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHTTQTSTSDNNTTHLSSMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPCENTDFRVLELYSQKELRSSNVFDLEEICREFNSYKQRVKYVEDKVVDRMKQLEDKVEELLSKNYHLENEVARLKKLVGERENLYFQGGHHHHHH

SEQ ID NO: 12 - protein, 33 aa, synthetic construct
RIKELEDRVEELLSELKHLEQEVARLKKLVGER

SEQ ID NO: 13 - protein, 33 aa, synthetic construct
RIKQLEDKVEELLSKLKHLEQEVARLKKLVGER

SEQ ID NO: 14 - protein, 33 aa, synthetic construct
RIKQLEDRVEELLSKLKHLEQEVARLKKLVGER

SEQ ID NO: 15 - protein, 33 aa, synthetic construct
RIKQLEDRVDELLSKLKHLEQEVARLKKLVGER

SEQ ID NO: 16 - protein, 33 aa, synthetic construct; identical to SEQ ID NO: 14

SEQ ID NO: 17 - protein, 33 aa, synthetic construct
RLKQLEDKVEELLSKLYHLEQEVARLKKLVGER

SEQ ID NO: 18 - protein, 201 aa, synthetic construct
MEELFKKHKIVAVLRANSVEEAKEKALALFEGGVHLIEITFTVPDADKVIKELSFLKEKGAIIGAGTVTSVEQCRKAICSGAEFIVSPHLDEEISRFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 19 - protein, 201 aa, synthetic construct
MEELFKKHKIVAVLRANSVEEAKEKALAVFEGGVHLIEITFTVPDADTVIKELSFLKEKGAIIGAGTVTSVEQCRKACESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 20 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSYKPINEDLDEGIMVVYKRNIVAHTFKVRIYQKVLTFRRSYAYHRTTYLLGSNTEYVAPPMWEIHHINSHGQCYSSYSRVIAGTVFVYYHRDSYENKTMQLMPDCYSNTHSTRYVTVKDQWHSRGSTRLYRETCNVNCMVTITTARSKYPYHFFVTSTGDVVDISPFYNGTNRNASYFGENADKFFIFPNYTIVSDFGRPNAALETHRLVAFLERADSVISWDIQDEKNVTCQLTFWEASERVIRSENEDSYHFISAKMTATFLTKKQEFNMSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRTYINQALAQIAEAWCVDQRRTLEIWKELSKINPSAILSAIYNRPIAARFMGDVLALSSCVTINQTSVKVLRDMNVKEDPGRCYSRPVVIFRFSNCSYVQYGQLGEDNEILLGNHRTEECQLPSLKIFIAGNQAYEYVDYLFKRMIDLSSISTVDSMIDLNIDPLENTDFRVLELYSQKELRSSNVFDLEEIMREFNSYKQRIKYVEDKVVDRIKELEDRVEELLSELKHLEQEVARLKKLVGER

SEQ ID NO: 21 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSYKPINEDLDEGIMVVYKRNIVAHTFKVRIYQKVLTFRRSYAYHRTTYLLGSNTEYVAPPMWEIHHINSHGQCYSSYSRVIAGTVFVYYHRDSYENKTMQLMPDCYSNTHSTRYVTVKDQWHSRGSTRLYRETCNVNCMVTITTARSKYPYHFFVTSTGDVVDISPFYNGTNRNASYFGENADKFFIFPNYTIVSDFGRPNAALETHRLVAFLERADSVISWDIQDEKNVTCQLTFWEASERVIRSENEDSYHFISAKMTATFLTKKQEFNMSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRTYINQALAQIAEAWCVDQRRTLEIWKELSKINPSAILSAIYNRPIAARFMGDVLALSSCVTINQTSVKVLRDMNVKEDPGRCYSRPVVIFRFSNCSYVQYGQLGEDNEILLGNHRTEECQLPSLKIFIAGNQAYEYVDYLFKRMIDLSSISTVDSMIDLNIDPLENTDFRVLELYSQKELRSSNVFDLEEIMREFNSYKQRIKYVEDKVVDRIKELEDRVEELLSELKHLEQEVARLKKLVGER

SEQ ID NO: 22 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 23 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 24 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGCDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNAALETHRLVAFLERADSVISWDIHDEKNVTCQLTFWEASERVIRSEAEDSYHFISAKMTATFLTKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKINPSAILSAIYNKPIAARFMGDVLALSSCVTINQTSVKVLRDMNVKEDPGRCYSRPVVIFRFSNSSYVQYGQLGEDNEILLGNHRTEECQLPSLKIFIAGNQAYEYVDYLFKRMIDLSSISTVDSMISLNIDPLENTDFRVLELYSQKELRSSNVFDLEEIMREFNSYKQRIKYVEDKVVDRIKQLEDRVEELLSKLKHLEQEVARLKKLVGER

SEQ ID NO: 25 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGCDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNAALETHRLVAFLERADSVISWDIHDEKNVTCQLTFWEASERVIRSEAEDSYHFISAKMTATFLTKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKINPSAILSAIYNKPIAARFMGDVLALSSCVTINQTSVKVLRDMNVKEDPGRCYSRPVVIFRFSNSSYVQYGQLGEDNEILLGNHRTEECQLPSLKIFIAGNQAYEYVDYLFKRMIDLSSISTVDSMISLNIDPLENTDFRVLELYSQKELRSSNVFDLEEIMREFNSYKQRIKYVEDKVVDRIKQLEDRVEELLSKLKHLEQEVARLKKLVGER

SEQ ID NO: 26 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGCDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 27 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGCDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 28 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTCDVVDISPFYNGTNRNASYFGENADKFFIFPNYTIVSDFGRPNSALETHRLVAFLERADSVISWDIQDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKINPSAILSAIYNKPIAARFMGDVLALSSCVTINQTSVKVLRDMNVKEDPGRCYSRPVVIFRFSNSSYVQYGQLGEDNEILLGNHRTEECQLCSLKIFIAGNQAYEYVDYLFKRMIDLSSISTVDSMISLNIDPLENTDFRVLELYSQKELRSSNVFDLEEIMREFNSYKQRIKYVEDKVVDRIKQLEDRVEELLSKLKHLEQEVARLKKLVGER

SEQ ID NO: 29 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTCDVVDISPFYNGTNRNASYFGENADKFFIFPNYTIVSDFGRPNSALETHRLVAFLERADSVISWDIQDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKINPSAILSAIYNKPIAARFMGDVLALSSCVTINQTSVKVLRDMNVKEDPGRCYSRPVVIFRFSNSSYVQYGQLGEDNEILLGNHRTEECQLCSLKIFIAGNQAYEYVDYLFKRMIDLSSISTVDSMISLNIDPLENTDFRVLELYSQKELRSSNVFDLEEIMREFNSYKQRIKYVEDKVVDRIKQLEDRVEELLSKLKHLEQEVARLKKLVGER

SEQ ID NO: 30 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTCDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLCSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 31 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTCDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLCSLKIFIAGNSAYEYVDYLFKRMIDLSSISTVDSMIALDIDPLENTDFRVLELYSQKELRSSNVFDLEEIMREFNSYKQRVKYVEDKVVDRMKQLEDKVEELLSKNYHLENEVARLKKLVGER

SEQ ID NO: 32 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTATFLSKKQEVNMSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEVWKELSKINPSAILSAIYNKPIAARFMGDVLGLASCVTINQTSVKVLRDMNVKESPGRCYSRPVVIFRFANSSYVQYGQLGEDNEILLGNHRTEECQLPSLKIFIAGNQAYEYVDYLFKRMIDLSSISTVDSMIALDIDPCENTDFRVLELYSQKELRSSNVFDLEEIMREFNSYKQRVKYVEDKVVDRLKQLEDKVEELLSKLYHLEQEVARLKKLVGER

SEQ ID NO: 33 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTATFLSKKQEVNMSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEVWKELSKINPSAILSAIYNKPIAARFMGDVLGLASCVTINQTSVKVLRDMNVKESPGRCYSRPVVIFRFANSSYVQYGQLGEDNEILLGNHRTEECQLPSLKIFIAGNQAYEYVDYLFKRMIDLSSISTVDSMIALDIDPCENTDFRVLELYSQKELRSSNVFDLEEIMREFNSYKQRVKYVEDKVVDRLKQLEDKVEELLSKLYHLEQEVARLKKLVGER

SEQ ID NO: 34 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPCENTDFRVLELYSQKELRSSNVFDLEEIMREFNSYKQRVKYVEDKVVDRMKQLEDKVEELLSKNYHLENEVARLKKLVGER

SEQ ID NO: 35 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPCENTDFRVLELYSQKELRSSNVFDLEEIMREFNSYKQRVKYVEDKVVDRMKQLEDKVEELLSKNYHLENEVARLKKLVGER

SEQ ID NO: 36 - protein, 736 aa, unidentified
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKICPSAILSAIYNKPIAARFMGDVLALSSCVTINQTSVKVLRDMNVKEDPGRCYSRPVVIFRFANSSYVQYGQLGEDNEILLGNHRTEECQLPSLKIFIAGNQAYEYVDYLFKRMIDLSSISTVDSMIALDIDPLENTDFRVLELYSQKELRSSNVFDLEEICREFNSYKQRIKYVEDKVVDRIKQLEDKVEELLSKLKHLEQEVARLKKLVGER

SEQ ID NO: 37 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKICPSAILSAIYNKPIAARFMGDVLALSSCVTINQTSVKVLRDMNVKEDPGRCYSRPVVIFRFANSSYVQYGQLGEDNEILLGNHRTEECQLPSLKIFIAGNQAYEYVDYLFKRMIDLSSISTVDSMIALDIDPLENTDFRVLELYSQKELRSSNVFDLEEICREFNSYKQRIKYVEDKVVDRIKQLEDKVEELLSKLKHLEQEVARLKKLVGER

SEQ ID NO: 38 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 39 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 40 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDCYSNTHSTRYVTVKDQWHSRGSTRLYRETCNLNCMVTITTARSKYPYHFFVTSTGDVVDISPFYNGTNRNASYFGENADKFFIFPNYTIVSDFGRPNAALETHRLVAFLERADSVISWDIHDEKNVTCQLTFWEASERVIRSEAEDSYHFISAKMTATFLTKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFENCSYVQYGQLGEDNEILLGNHRTEECQLPSLKIFIAGNQAYEYVDYLFKRMIDLSSISTVDSMISLNIDPLENTDFRVLELYSQKELRSSNVFDLEEICREFNSYKQRIRYVEDKVVDRIKQLEDRVDELLSKLKHLEQEVARLKKLVGER

SEQ ID NO: 41 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDCYSNTHSTRYVTVKDQWHSRGSTRLYRETCNLNCMVTITTARSKYPYHFFVTSTGDVVDISPFYNGTNRNASYFGENADKFFIFPNYTIVSDFGRPNAALETHRLVAFLERADSVISWDIHDEKNVTCQLTFWEASERVIRSEAEDSYHFISAKMTATFLTKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFENCSYVQYGQLGEDNEILLGNHRTEECQLPSLKIFIAGNQAYEYVDYLFKRMIDLSSISTVDSMISLNIDPLENTDFRVLELYSQKELRSSNVFDLEEICREFNSYKQRIRYVEDKVVDRIKQLEDRVDELLSKLKHLEQEVARLKKLVGER

SEQ ID NO: 42 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 43 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTRLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 44 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGCDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFSNSSYVQYGQLGEDNEILLGNHRTEECQLPSLKIFIAGNQAYEYVDYLFKRMIDLSSISTVDSMISLNIDPLENTDFRVLELYSQKELRSSNVFDLEEICREFNSYKQRIKYVEDKVVDRIKQLEDRVEELLSKLKHLEQEVARLKKLVGER

SEQ ID NO: 45 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGCDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFSNSSYVQYGQLGEDNEILLGNHRTEECQLPSLKIFIAGNQAYEYVDYLFKRMIDLSSISTVDSMISLNIDPLENTDFRVLELYSQKELRSSNVFDLEEICREFNSYKQRIKYVEDKVVDRIKQLEDRVEELLSKLKHLEQEVARLKKLVGER

SEQ ID NO: 46 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGCDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 47 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGCDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFCTSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 48 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTCDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFENSSYVQYGQLGEDNEILLGNHRTEECQLCSLKIFIAGNQAYEYVDYLFKRMIDLSSISTVDSMISLNIDPLENTDFRVLELYSQKELRSSNVFDLEEICREFNSYKQRIKYVEDKVVDRIKQLEDRVDELLSKLKHLEQEVARLKKLVGER

SEQ ID NO: 49 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTSTCDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRKYINRALAQIAEAWCVDQRRTLEVWKELSKICPSAILSAIYNKPIAARFMGDVLALSSCVTINQTSVKVLRDMNVKEDPGRCYSRPVVIFRFENSSYVQYGQLGEDNEILLGNHRTEECQLCSLKIFIAGNQAYEYVDYLFKRMIDLSSISTVDSMISLNIDPLENTDFRVLELYSQKELRSSNVFDLEEICREFNSYKQRIKYVEDKVVDRIKQLEDRVDELLSKLKHLEQEVARLKKLVGER

SEQ ID NO: 50 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTCDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLCSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 51 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATSTCDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLCSLKIFIAGNSAYEYVDYLFKRMIDLSSISTVDSMIALDIDPLENTDFRVLELYSQKELRSSNVFDLEEICREFNSYKQRVKYVEDKVVDRMKQLEDKVEELLSKNYHLENEVARLKKLVGER

SEQ ID NO: 52 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTCTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKICPSAILSAIYNKPIAARFMGDVLALSSCVTINQTSVKVLRDMNVKEDPGRCYSRPVVIFRFENSSYVQYGQLGEDNEILLGNHRTEECQLPSLKIFIAGNQAYEYVDYLFKRMIDLSSISTVDSMISLNIDPCENTDFRVLELYSQKELRSSNVFDLEEICREFNSYKQRIKYVEDKVVDRIKQLEDRVEELLSKLKHLEQEVARLKKLVGER

SEQ ID NO: 53 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGGDLFRFERNIVCTSMKPINEDLDEGIMVVYKRNIVAHTFKVRIYQKVLTFRRSYAYHRTTYLLGSNTEYVAPPMWEIHHINSHGQCYSSYSRVIAGTVFVAYHRDSYENKTMQLMPDDYSNTHSTRYVTVKDQWHSRGSTRLYRETCNLNCMVTITTARSKYPYHFFVTCTGDVVDISPFYNGTNRNASYFGENADKFFIFPNYTIVSDFGRPNSALETHRLVAFLERADSVISWDIHDEKNVTCQLTFWEASERVIRSEAEDSYHFISAKMTATFLSKKQEVNMSDSALDCVRDEAINKLQQIFNTSYNQTYEKYGNVSVFETTGGLFVFWQPIKQKSLVELERLANRSSLNLTHNSTKSSTDGNNATHLSNMESVHNLVYAQLQFTYDTLRAYINRALAQIAEAWCVDQRRTLEVWKELSKICPSAILSAIYNKPIAARFMGDVLALSSCVTINQTSVKVLRDMNVKEDPGRCYSRPVVIFRFENSSYVQYGQLGEDNEILLGNHRTEECQLPSLKIFIAGNQAYEYVDYLFKRMIDLSSISTVDSMISLNIDPCENTDFRVLELYSQKELRSSNVFDLEEICREFNSYKQRIKYVEDKVVDRIKQLEDRVEELLSKLKHLEQEVARLKKLVGER

SEQ ID NO: 54 - protein, 736 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPCENTDFRVLELYSQKELRSSNVFDLEEICREFNSYKQRVKYVEDKVVDRMKQLEDKVEELLSKNYHLENEVARLKKLVGER

SEQ ID NO: 55 - protein, 714 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTRLYRETCNLNCMVTITTARSKYPYHFFATCTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKICPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPCENTDFRVLELYSQKELRSSNVFDLEEICREFNSYKQRVKYVEDKVVDRMKQLEDKVEELLSKNYHLENEVARLKKLVGER

SEQ ID NO: 56 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMATGGDLVRFERNIVCTSMKPINEDLDEGIMVVYKRNIVAHTFKVRVYQKVLTFRRSYAYIHTTYLLGSNTEYVAPPMWEIHHINKHGQCYSSYSRVIAGTVFVYYHRDSYENKTMQLMPDDYSNTHSTRYVTVKDQWHSRGSCGLYRETCTLNCMVTIVTARSKYPYHFFVLSTGDIVDISPFYDGTNRNASYFGENADKFFIFPNYTIVSDFGRPNAALETHRLVAFLERADSVISWDIQDEKNVTCQLTFWDASERTIRTETEDSYHFISAKMTATFIAKKQEVNESDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEIWKELSKINPSAILSAIYNRPIAARWLGDVLGISSCVTINQTSVKVLRDMNVKEDPGRCYSRPVVIFQFANSSYVFYGQLGEDNEILLGNHRTEECQLPSLKIFITGNSAYEYVDYLFKRMIDLSSISTVDSMIDLDIDPLENTDFRLLELYSQKELRSSNVFDLEEIMREFNSYKQRVKEIEDKVVDNLPPYLQGLDSLMSDLGTVGKMEELFKKHKIVAVLRANSVEEAKEKALALFEGGVHLIEITFTVPDADKVIKELSFLKEKGAIIGAGTVTSVEQCRKAICSGAEFIVSPHLDEEISRFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 57 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMATGGDLVRFERNIVCTSMKPINEDLDEGIMVVYKRNIVAHTFKVRVYQKVLTFRRSYAYIHTTYLLGSNTEYVAPPMWEIHHINKHGQCYSSYSRVIAGTVFVYYHRDSYENKTMQLMPDDYSNTHSTRYVTVKDQWHSRGSCGLYRETCTLNCMVTIVTARSKYPYHFFVLSTGDIVDISPFYDGTNRNASYFGENADKFFIFPNYTIVSDFGRPNAALETHRLVAFLERADSVISWDIQDEKNVTCQLTFWDASERTIRTETEDSYHFISAKMTATFIAKKQEVNESDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEIWKELSKINPSAILSAIYNRPIAARWLGDVLGISSCVTINQTSVKVLRDMNVKEDPGRCYSRPVVIFQFANSSYVFYGQLGEDNEILLGNHRTEECQLPSLKIFITGNSAYEYVDYLFKRMIDLSSISTVDSMIDLDIDPLENTDFRLLELYSQKELRSSNVFDLEEIMREFNSYKQRVKEIEDKVVDNLPPYLQGLDSLMSDLGTVGKMEELFKKHKIVAVLRANSVEEAKEKALALFEGGVHLIEITFTVPDADKVIKELSFLKEKGAIIGAGTVTSVEQCRKAICSGAEFIVSPHLDEEISRFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 58 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSCWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKAVCSGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 59 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSCWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKAVCSGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 60 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMATGGDLVRFERNIVCTSMKPINEDLDEGIMVVYKRNIVAHTFKVRVYQKVLTFRRSYAYCHTTYLLGSNTEYVAPPMWEIHHINKHGQCYSSYSRVIAGTVFVYYHRDSYENKTMQLMPDDYSNTHSTRYVTVKDQWHSRGSTGLYRETCTLNCMVTITEARSKYPYHFFVLSTGDIVDISPFYDGTNRNASYFGENADKFFIFPNYTIVSDFGRPNAALETHRLVAFLERADSVISWDIQDEKNVTCQLTFWDASERTIRSETEDSYHFISAKMTATFVAKKQEVNPSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEIWKELSKINPSAILSAIYNRPIAARWLGDVLGISSCVTINQTSVKVLRDMNVKEDPGRCYSRPVVIFQFANSSYVFYGQLGEDNEILLGNHRTEECQLPSLKIFITGNSAYEYVDYLFKRMIDLSSISTVDSMIDLDIDPLENTDFRLLELYSQKELRSSNVFDLEEIMREFNSYKQRVKEVEDKVVDNLPPYLQGLDSLMDDLGSVGKMEELFKKHKIVAVLRANSVEEAKEKALALFEGGVHLIEITFTVPDADKVIKELSFLKEKGAIIGAGTVTSVEQCRKACESGAEFIVSPHLDEEISRFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 61 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMATGGDLVRFERNIVCTSMKPINEDLDEGIMVVYKRNIVAHTFKVRVYQKVLTFRRSYAYCHTTYLLGSNTEYVAPPMWEIHHINKHGQCYSSYSRVIAGTVFVYYHRDSYENKTMQLMPDDYSNTHSTRYVTVKDQWHSRGSTGLYRETCTLNCMVTITEARSKYPYHFFVLSTGDIVDISPFYDGTNRNASYFGENADKFFIFPNYTIVSDFGRPNAALETHRLVAFLERADSVISWDIQDEKNVTCQLTFWDASERTIRSETEDSYHFISAKMTATFVAKKQEVNPSDSALDCVRDEAINKLQQIFNTSYNQTYEKYGNVSVFETTGGLIVFWQPIKQKSLVELERLANRSSLNLTHNSTKSSTDGNNATHLSNMESVHNLVYAQLQFTYDTLRKYINRALAQIAEAWCVDQRRTLEIWKELSKINPSAILSAIYNRPIAARWLGDVLGISSCVTINQTSVKVLRDMNVKEDPGRCYSRPVVIFQFANSSYVFYGQLGEDNEILLGNHRTEECQLPSLKIFITGNSAYEYVDYLFKRMIDLSSISTVDSMIDLDIDPLENTDFRLLELYSQKELRSSNVFDLEEIMREFNSYKQRVKEVEDKVVDNLPPYLQGLDSLMDDLGSVGKMEELFKKHKIVAVLRANSVEEAKEKALALFEGGVHLIEITFTVPDADKVIKELSFLKEKGAIIGAGTVTSVEQCRKACESGAEFIVSPHLDEEISRFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 62 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYCHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKACESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 63 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYCHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKACESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 64 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMANGGDLIRFERNIVCTSMKPINEDLDEGIMVVYKRNIVAHTFKVRVYQKVLTFRRSYAYIHTTYLLGSNTEYVAPPMWEIHHINRHGQCYSSYSRVIAGTVFVAYHRDSYENKTMQLMPDCYSNTHSTRYVTVKDQWHSRGSTGLYRETCNLNCMVTIVTARSKYPYHFFALSTGDVVDISPFYDGTNRNASYFGENADKFFIFPNYTIVSDFGRPNSALETHRLVAFLERADSVISWDIQDEKNVTCQLTFWDASERTIRSETEDSYHFISAKMTATFLSKKQEVNMSDSALDCVRDEAINKLQQIFNTSYNQTYEKYGNVSVFETTGGLVVFWQPIKQKSLVELERLANRSSLNLTHNSTKSSTDGNNATHLSNMESVHNLVYAQLQFTYDTLREYINRALAQIAEAWCVDQRRTLEVWKELSKINPSAILSAIYNKPIAARFMGDVLGISSCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIDLDIDPLENTDFRVLELYSQKELRSSNVFDLEEIMREFNSYKQRVKYVEDKVVDNLPPYLKGLDSLMSDLGDVGKMEELFKKHKIVAVLRANSVEEAKEKALALFEGGVHLIEITFTVPDADKVIKELSFLKEKGAIIGAGTVTSVEQCRKAVESGAEFIVSPHLDEEISQFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 65 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMANGGDLIRFERNIVCTSMKPINEDLDEGIMVVYKRNIVAHTFKVRVYQKVLTFRRSYAYIHTTYLLGSNTEYVAPPMWEIHHINRHGQCYSSYSRVIAGTVFVAYHRDSYENKTMQLMPDCYSNTHSTRYVTVKDQWHSRGSTGLYRETCNLNCMVTIVTARSKYPYHFFALSTGDVVDISPFYDGTNRNASYFGENADKFFIFPNYTIVSDFGRPNSALETHRLVAFLERADSVISWDIQDEKNVTCQLTFWDASERTIRSETEDSYHFISAKMTATFLSKKQEVNMSDSALDCVRDEAINKLQQIFNTSYNQTYEKYGNVSVFETTGGLVVFWQPIKQKSLVELERLANRSSLNLTHNSTKSSTDGNNATHLSNMESVHNLVYAQLQFTYDTLREYINRALAQIAEAWCVDQRRTLEVWKELSKINPSAILSAIYNKPIAARFMGDVLGISSCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIDLDIDPLENTDFRVLELYSQKELRSSNVFDLEEIMREFNSYKQRVKYVEDKVVDNLPPYLKGLDSLMSDLGDVGKMEELFKKHKIVAVLRANSVEEAKEKALALFEGGVHLIEITFTVPDADKVIKELSFLKEKGAIIGAGTVTSVEQCRKAVESGAEFIVSPHLDEEISQFCKEKGVFYMPGVMTPTELVKALKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 66 - protein, 925 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 67 - protein, 903 aa, synthetic construct
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC